(19) United States
(12) Patent Application Publication
(10) Pub. No.: US 2006/0035813 A1
(43) Pub. Date: Feb. 16, 2006

(54) Methods and formulations comprising agonists and antagonists of nuclear hormone receptors

(76) Inventors: Esther M. Sternberg, WASHINGTON, DC (US); Jeannette I. Webster, Washington, DC (US); Leonardo H. Tonelli, Bethesda, MD (US); Stephen H. Leppla, Bethesda, MD (US); Mahtab Moayeri, Bethesda, MD (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(21) Appl. No.: 10/530,254

(22) PCT Filed: Oct. 3, 2003
(86) PCT No.: PCT/US03/31406

§ 371 Date: Apr. 4, 2005

Related U.S. Application Data
(60) Provisional application No. 60/416,222, filed on Oct. 4, 2002.
(60) Provisional application No. 60/419,454, filed on Oct. 18, 2002.

(51) Int. Cl. A61K 38/28

(52) U.S. Cl. 514/3

(57) ABSTRACT

Novel compounds, pharmaceutical compositions, and methods are provided for modulating processes mediated by nuclear hormone receptors. A partial or complete agonist or antagonist modulates, directly or indirectly, an activity of one or more nuclear hormone receptors for glucocorticoids (GRs), androgens (ARs), mineralocorticoids (MRs), progestins (PRs), estrogens (ERs), thyroid hormones (TRs), vitamin D (VDRs), retinoids (RARs and RXRs), peroxisomes (XPARs and PPARs), icosanoids (IRs), or one or more orphan receptors, such as steroid and thyroid receptors. Exemplary compounds of the disclosure are bacterial products, for example bacterial toxins, and these compounds are useful in screens for other antagonists and agonists. Related methods and compositions are provided for diagnosis, treatment and prevention of bacterial disease and associated or unrelated inflammatory, autoimmune, toxic (including shock), and chronic and/or lethal sequelae associated with bacterial infection.

### PRIORITY CLAIM

This application claims the benefit of U.S. Provisional Application No. 60/416,222, filed Oct. 4, 2002, and U.S. Provisional Application No. 60/419,454, filed Oct. 18, 2002. Both of these provisional applications are incorporated herein in their entirety.

### FIELD

This application relates to methods for identifying agonists and antagonists of a nuclear hormone receptor using bacterial products.

### BACKGROUND

The effectiveness of known modulators of steroid receptors is often compromised by their undesired side-effect profile, particularly after long-term administration. For example, the effectiveness of progesterone and estrogen agonists, such as norgestrel and diethylstilbesterol respectively, as female birth control agents must be weighed against the increased risk of breast cancer and heart disease to women taking such agents. Similarly, the progesterone antagonist, mifepristone (RU486), if administered for chronic indications, such as uterine fibroids, endometriosis and certain hormone-dependent cancers, could lead to homeostatic imbalances in a patient due to its inherent cross-reactivity as a GR antagonist. Accordingly, identification of additional compounds and methods for modulating activity of nuclear hormone receptors will be of significant value in the treatment of a wide range of diseases.

Although there are compositions and methods proposed in the art for modulating nuclear hormone receptor activity and thereby ameliorating disease mediated directly or indirectly by the action of nuclear hormone receptors, there is a continuing need for and a continuing search in the field for additional and more effective compositions and methods to satisfy these objectives. Thus, the identification of compounds and methods that effectively modulate nuclear hormone receptor activity with minimal side effects remains an important objective in the art.

### SUMMARY

Compounds, pharmaceutical compositions, and methods for modulating processes mediated by nuclear hormone receptors are provided herein.

In one embodiment, methods are provided for identifying a compound that has an effect of a partial or complete agonist or antagonist of one or more nuclear hormone receptors for glucocorticoids (GRs), androgens (ARs), mincralocorticoids (MRs), progestins (PRs), estrogens (ERs), thyroid hormones (TRs), vitamin D (VDRs), retinoids (RARs and RXRs), peroxisomes (XPARs and PPARs), icosanoids (IRs), or one or more orphan receptors, such as steroid and thyroid receptors.

Methods and pharmaceutical compositions are provided for treatment and prevention of bacterial disease and associated or unrelated inflammatory, autoimmune, toxic (including shock), and chronic and/or lethal sequelae associated with bacterial infection. In related aspects, methods and pharmaceutical compositions are provided for treatment and prevention inflammatory, autoimmune immunological, lethal and toxic symptoms and diseases not causally associated with bacterial infection. These methods and compositions generally employ one or more agonists or antagonists of a nuclear hormone receptor as described herein.

Methods are further provided for identifying a compound that has the effect of an agonist or antagonist of a nuclear hormone receptor. In exemplary embodiments these methods generally include the steps of providing viable cells that express a nuclear hormone receptor and a nuclear hormone receptor reporter construct, wherein expression of the substrate reporter construct is detectable and provides a measurement of nuclear hormone receptor pathway activation or repression. Test cells are contacted with a test agent and a bacterial product, and control cells are contacted with the bacterial product alone. Then nuclear hormone receptor pathway activation or repression is detected and compared between the test and control cells to identify a test agent that modulates activation or repression of the nuclear hormone receptor pathway activity by the bacterial product. Related methods are provided for identifying cofactors, nuclear hormone receptors and other useful agents that interact directly or indirectly with bacterial products to mediate activation or repression of nuclear hormone receptors.

### BRIEF DESCRIPTION OF THE FIGURES

FIGS. 1A and 1B are two graphs showing anthrax lethal toxin (LeTx) repression of Dexamethasone-(Dex)-induced glucocorticoid receptor (GR) transactivation in Cos7 cells. Cos7 cells were plated out at a density of 5×10⁵ cells/well in 24-well plates in Dulbecco's modified Eagle's medium (DMEM) containing 10% charcoal-stripped serum, 10 μg/1 ml penicillin-streptomycin and 2 mM glutamine one day prior to transfection. Cos7 cells were transfected overnight with 20 ng SV glucocorticoid receptor (SVGR), 100 ng glucocorticoid response element-luciferase reporter construct (GRE-TK luc), 60 ng pSG5 (Stratagene) and 20 ng PRL TK (Promega, constitutive renilla luciferase control) using Fugene6 (Roche) according to Manufacturer's instructions. The media was then replaced with DMEM containing 10% charcoal-stripped media, 100 nM Dex and either with increasing concentrations of LF, alone (●) or in the presence of 500 ng/ml PA (◯) (FIGS. 1A and 1B); or with increasing concentrations of E687C, either alone (▪) or in the presence of 500 ng/ml PA (□) (1B). After 24 hours the cells were lysed and firefly and renilla luciferase assayed using the dual luciferase assay (Promega). The firefly luciferase activity was normalized to the renilla luciferase to control for differences in cell number and transfection efficiency. The induction was calculated as the mean of triplicate normalized luciferase samples in the presence of 100 nM Dex divided by the mean normalized luciferase in the absence of Dex. In order to compare separate experiments the induction was set to 100% for 100 nM Dex treatment only (for example, no lethal factor (LF) or protective antigen (PA)) and the other data points normalized to this accordingly. The data shown is the mean and standard deviation of eight (FIG. 1A) or three (FIG. 1B) experiments. A one-way analysis of variance (ANOVA) followed by a Dunnett post hoc test was performed between 100 nM Dex only treatments and 100 nM Dex and LF and/or PA treatments. A single asterisk (*) designates a p value of 0.01-0.05; a double asterisk (**) designates a p value of 0.001-0.01; a triple asterisk (***) designates a p value of <0.001.

FIGS. 2A and 2B are two graphs showing a comparison of the effects of mifepristone (RU 486) and LeTx on the dose response curve of Dex in GR-transfected cos7 cells. Cos7 cells were transfected as described for FIG. 1. After transfection, the media was replaced with DMEM containing 10% charcoal-stripped media and increasing concentrations of Dex, either alone (▪) or in the presence of 0.2 μM RU 486 (□), 500 ng/ml PA and 5 ng/ml LF (●) or 50 ng/ml LF and 10 ng/ml PA (◯). After twenty-four hours the cells were lysed as described above. The mean and standard deviation of three experiments are shown in FIG. 2A. The renilla normalized luciferase data (standardized to 100 for 1 μM Dex in each individual experiment) is shown with the data normalized as a percentage of maximal for each treatment shown in FIG. 2B (inset).

FIG. 3 is a graph showing a comparison of the effects of LeTx on the mutant 407C and wild type GR. Cos7 cells were transfected using either the same plasmid mix as described in FIG. 1 containing the wild type GR (□) or with the mutant 407C GR, which lacks the N-terminal transactivation domain (◯). After transfection, the media was replaced with DMEM containing 10% charcoal-stripped media, 1 μM Dex, and increasing concentrations of the LF in the presence of 500 ng/ml PA. After 24 hours the cells were lysed and firefly luciferase values were normalized to renilla luciferase. Experiments were then compiled by standardization as described for FIG. 1. The mean +/− standard deviation of three experiments is shown. Statistics were performed using a one-way ANOVA followed by a Bonferroni post hoc test.

FIG. 4 is a graph showing LeTx repression of dexamethasone induced tyrosine aminotransferase (TAT) in hepatoma cell line (HTC) cells. HTC cells were plated out at a density of 5×10⁶ cells/plate in 6 cm plates in DMEM containing 10% fetal calf serum 10 μg/ml penicillin-streptomycin and 2 mM glutamine one day prior to treatment. The media was then replaced with DMEM containing increasing concentrations of Dex either alone (◯) or together with 2 ng/ml LF in the presence of 500 ng/ml PA (●) or with 10 ng/ml LF in the presence of 500 ng/m PA (▴). After 18 hours the cells were lysed by sonication and TAT activity assayed. The mean and standard deviations are shown and a one-way ANOVA followed by a Bonferroni post hoc test was performed.

FIGS. 5A, 5B, 5C, and 5D are four graphs showing a comparison of the effects of LeTx and inhibitors of MEK1 and JNK pathways on the response of a Dex-induced GRE luciferase and a constitutive luciferase. Cos7 cells were transfected with SVGR and (GRE)₂-TK luc (●) or with SVGR and the constitutive luciferase vector, pGL3 control (Promega) (□) and treated with 100 nM dexamethasone, and increasing concentrations LF with 500 ng/ml PA (FIG. 5), or increasing concentrations of the MEK1 inhibitors, PD98059 (FIG. 5B), and U0126 (FIG. 5C) or the JNK inhibitor, SP600126 (FIG. 5D). Means and standard deviations are shown and data was analyzed using a two-way ANOVA followed by a Scheffe post hoc test.

FIGS. 6A, 6B, 6C, 6D, 6E, and 6F are a set of graphs (FIGS. 6A, 6C and 6E) and digital images of gels (FIGS. 6B, 6D, and 6F) showing the effect of p38 MAP kinase inhibitors on the response of a Dex-induced GRE luciferase and a constitutive luciferase and on inhibition of p38. Cos7 cells were transfected with SVGR and (GRE)₂-TK luc (▪) or with SVGR and the constitutive luciferase vector, pGL3 control (□) and treated 100 nM dexamethasone, and increasing concentrations of the p38 MAP kinase inhibitors, SB203580 (FIG. 6A), SB220025 (FIG. 6C) and p38 MAP kinase inhibitor (FIG. 6E). Means and standard deviations are shown and data was analyzed using a two-way ANOVA followed by a Scheffe post hoc test. Cos7 cells were pre-treated for 30 nin with various concentrations of SB203580 (FIG. 6B), SB220025 (FIG. 6D) or p38 MAP kinase inhibitor (FIG. 6F) and then further incubated with 10 μg/ml anisomycin for 30 min. Proteins were then subjected to SDS-PAGE and Western blotting using an anti-phospho-p38 antibody.

FIGS. 7A, 7B, 7C, and 7D are a set of four graphs showing the effects of the LeTx on hormone-induced activity of other nuclear hormone receptors in cos7 cells. Cos7 cells were transfected as described for FIG. 1. except that 20 ng expression plasmids for MR (FIG. 7A), ERα (FIG. 7B), ERβ (FIG. 7C) or PR-B (FIG. 7D) were used. One hundred ng of the firefly luciferase reporters, GRE-luc (FIG. 7A), 100 ng ERE-luc (FIGS. 7B and 7C) or pHr-luc (FIG. 7D) were used. After transfection, the media was replaced with DMEM containing 10% charcoal-stripped media and 100 nM aldosterone (FIG. 7A), 1 nM 17β-estradiol (FIG. 7B), 100 nM 17β-estradiol (FIG. 7C), or 100 nM progesterone (FIG. 7D), either containing increasing concentrations of LF alone (▪) or in the presence of 500 ng/ml PA (□). After 24 hours the cells were lysed and data analyzed as described earlier. The mean and standard deviation of five experiments are shown.

FIG. 8 is a digital image of a gel showing that LF and PA do not affect GR binding to a GRE probe in a gel shift experiment Twenty-five μg of GR-transfected cos7 cytosol was incubated with a [³²P] labeled GRE probe in the presence of 40 fold excess unlabeled probe as a competitor or with 5, 10 or 50 ng/ml LF, 10, 50 or 500 ng/ml PA, or with 5, 10 or 50 ng/ml LF in the presence of 500 ng/ml PA. The samples were run on a 40% Tris-borate-EDTA (TBE) acrylamide gel and visualized by autoradiography.

FIG. 9 is a graph showing, that PA and/or LF do not prevent [³H] dexamethasone binding to GR transfected cos7 cell cytosol preparations. One hundred μg GR transfected cos7 cytosol was incubated overnight with 10 nM [³H] dexamethasone in the presence or absence of 500 fold excess unlabeled dexamethasone and in the presence of 1 μM RU486, 500 ng/ml PA, 50 ng/ml LF or 500 ng/ml PA+50 ng/ml LF. Bound was separated from free and specific binding calculated. The percent specific binding in comparison to dexamethasone alone is shown.

FIG. 10 is a graph showing that RU486 can fully repress dexamethasone-induced GR transactivation and progesterone-induced PR-B transactivation in cos7 cells even in the presence of LeTx. Cos7 cells were transfected with SVGR and (GRE)₂-TK luc or PR-B and pLTR luc and then treated with 100 nM dexamethasone or progesterone in the presence of 2 ng/ml LF+500 ng/ml PA and increasing concentrations of RU486 (maximum 1 μM). Relative luciferase values were measured.

FIG. 11 is a graph showing LeTx repression of dexamethasone induced tyrosine aminotransferase (TAT) in mouse livers. BALB/cJ mice were injected with LeTx and 30 minutes later with Dex. After six and twelve hours liver TAT activity was assayed. Means and standard deviations of six to ten animals are shown and a two-way ANOVA followed by a Scheffe post hoc test was performed.

FIG. 12 is a schematic diagram showing the structure of the various MR/GR chimeras and an indication as to whether these are repressed by LeTx on the (GRE)₂ TK luc promoter.

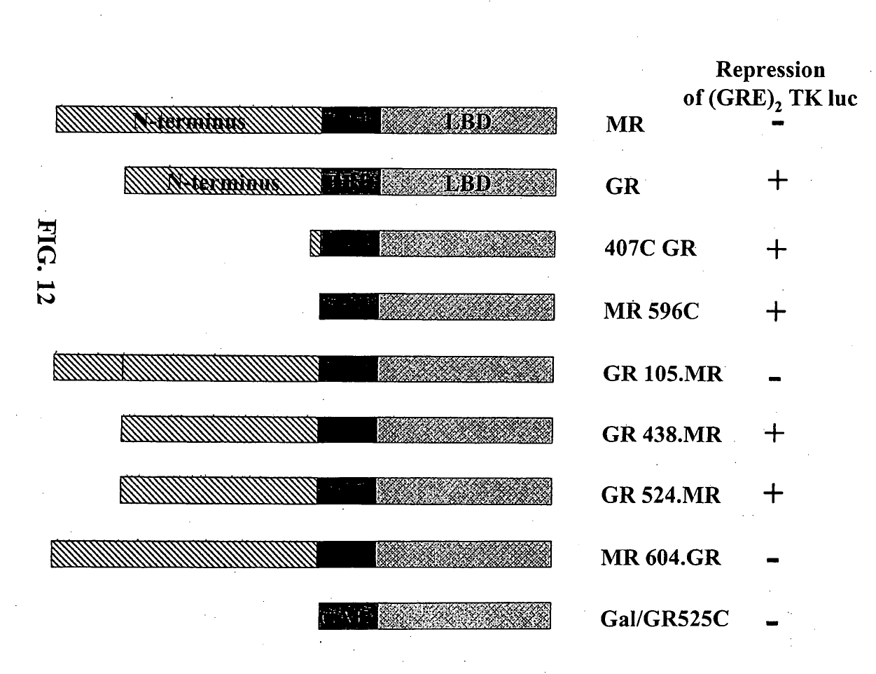

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO:1 is the amino acid sequence of human immunodeficiency virus (HIV)-1 Tat protein.

### DETAILED DESCRIPTION

I. Abbreviations

ANOVA: analysis of variance
AR: androgen receptor
ATP: adenosine triphosphate
DBD: DNA binding domain
Dex: dexamethasone
DMEM: Dulbecco's modified Eagle's medium
ER: estrogen receptor
EDTA: ethylenediaminetetraacetic acid
GR: glucocorticoid receptor
GRE: glucocorticoid response element
GRE-TK luc: glucocorticoid response element-luciferase reporter construct
GS: glutamine synthase
HEPES: 4-(2-hydroxyethyl)-1-piperazineethanesulfonic acid
HTC: hepatoma cell line
IL-6: interleukin-6
IR: icosanoid receptor
LBD: ligand binding domain
LeTx: lethal toxin
LF: lethal factor
LPS: lipopolysaccharide
Luc: luciferase
MAPK: MAP Kinase
μg: microgram
μl: microliter
μM: micromolar
MR: mineralocorticoid receptor
MTT: 3,[4,5-dimethylthiazol-2-yl]-2,5-phenyltetrazolium bromide
NFκB: nuclear factor kappa B
ng: nanogram
nM: nanomolar
PA: protective antigen
PBS: phosphate buffered saline
PEPCK: phosphoenolpyruvate carboxykinase
PPAR: peroxisome receptor
PR-B: progesterone B receptor
PVDF: polyvinylidene fluoride
RAR: retinoid receptor
RU 486: mifepristone
RXR: retinoid receptor
SDS-PAGE: sodium dodecyl sulphate polyacrylamide-gel electrophoresis
SVGR: SV glucocorticoid receptor
TAT: tyrosine aminotransferase
TBE: Tris-borate-EDTA
TR: thyroid hormone receptor
TNF-α: tumor necrosis factor-α
VDR: vitamin D receptor
XPAR: peroxisonie receptor
   II. Description of Several Specific Embodiments

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

### Nuclear Hormone Receptors

Nuclear hormone receptors comprise a superfamily of proteins that includes receptors for glucocorticoids (GRs), androgens (ARs), mineralocorticoids (MRs), progestins (PRs), estrogens (ERs), thyroid hormones (TRs), vitamin D (VDRs), retinoids (RARs and RXRs), peroxisomes (XPARs and PPARs) and icosanoids (IRs). In addition, “orphan receptors,” such as steroid and thyroid receptors, are structurally related to classic nuclear hormone receptors and are considered part of the nuclear hormone receptor superfamily. Unlike integral membrane receptors and membrane-associated receptors, nuclear hormone receptors are located in the cytoplasm or nucleus of eukaryotic cells.

Nuclear hormone receptors are specifically bound and activated by physiologically important small molecule ligands. Ligands of nuclear hormone receptors include native hormones, such as progesterone, estrogen and testosterone, vitamins, as well as synthetic derivative compounds, such as medroxyprogesterone acetate, diethylstilbesterol and 19-nortestosterone. Nuclear hormone receptor ligands, when present in a physiological compartment surrounding a cell, pass through the outer cell membrane and bind to their cognate receptor with high affinity (commonly in the 0.01-20 nM range) to create an activated ligand/receptor complex. This complex translocates to the cell's nucleus where it binds to a specific gene or genes present in the cell's DNA. Once bound to DNA, the ligand/receptor complex modulates transcription of target genes and thereby regulates expression of specific proteins encoded by the target genes.

The activated nuclear hormone receptor/ligand complex functions to induce certain genes to initiate or increase transcriptional activity, and/or to suppress activity of other genes. Modulation of nuclear hormone receptor activity can therefore involve activation or inhibition of receptor function, which in turn can involve increased or decreased activities of gene induction and/or suppression. A compound that mimics the effect of a native ligand of a nuclear hormone receptor is referred to as an “agonist,” while a compound that inhibits the effect of a native ligand is called an “antagonist.”

Ligands to the steroid receptors are known to play an important role in health of both women and men. Excesses or deficiencies of these ligands can have profound physiological consequences. For example, an excess of the GR ligand glucocorticoid results in Cushing's Syndrome, while glucocorticoid deficiency is associated with Addison's Disease. The native ligand progesterone in females, as well as synthetic analogues, such as norgestrel (18-homonorethisterone) and norethisterone (17α-ethinyl-19-nortestosterone), are effective in birth control formulations, typically in combination with the female hormone estrogen or synthetic estrogen analogues, as modulators of both PR and ER. On the other hand, antagonists to PR are useful in treating hormone dependent cancers of the breast, ovaries, and uterus, and certain non-malignant conditions such as uterine fibroids and endometriosis, a leading cause of infertility in women. Similarly, AR antagonists, such as cyproterone acetate and flutamide have proved useful in the treatment of hyperplasia and cancer of the prostate.

The effectiveness of known modulators of steroid receptors is often compromised by their undesired side-effect profile, particularly after long-term administration. For example, the effectiveness of progesterone and estrogen agonists, such as norgestrel and diethylstilbesterol respectively, as female birth control agents must be weighed against the increased risk of breast cancer and heart disease to women taking such agents. Similarly, the progesterone antagonist, mifepristone (RU486), if administered for chronic indications, such as uterine fibroids, endometriosis and certain hormone-dependent cancers, could lead to homeostatic imbalances in a patient due to its inherent cross-reactivity as a GR antagonist. Accordingly, identification of additional compounds and methods for modulating activity of nuclear hormone receptors will be of significant value in the treatment of a wide range of diseases.

The glucocorticoid receptor (GR) is present in glucocorticoid responsive cells where it resides in the cytosol in an inactive state until stimulated by a GR agonist. Upon stimulation the receptor translocates to the cell nucleus where it specifically interacts with DNA and/or protein(s) and regulates transcription in a glucocorticoid responsive manner. Two examples of proteins that interact with the glucocorticoid receptor are the transcription factors, API and NFκ-B. Such interactions result in inhibition of API- and NF κ-B-mediated transcription and are believed to be responsible for some of the anti-inflammatory activity of endogenously administered glucocorticoids. In addition, glucocorficoids may also exert physiologic effects independent of nuclear transcription.

Biologically relevant glucocorticoid receptor agonists include cortisol and corticosterone. Many synthetic glucocorticoid receptor agonists exist including dexamethasone, prednisone, prednisolone, methylprednisolone, and trimcinolone. Glucocorticoid receptor antagonists, for example RU486, typically bind to the receptor and prevent glucocorticoid receptor agonists from binding and eliciting GR-mediated events.

The search for effective nuclear hormone receptor agonists and antagonists and related methods for modulating nuclear hormone receptor activity remains an important objective of academic, medical and industry research. In this context, U.S. Pat. No. 5,767,113 discloses certain synthetic steroid compounds that are reportedly useful for concurrently activating glucocorticoid-induced response and reducing multidrug resistance. Published European Patent Application 0 683 172, published Nov. 11, 1995, discloses certain 11,21-bisphenyl-19-norpregnane derivatives reportedly having anti-glucocorticoid activity useful to treat or prevent glucocorticoid-dependent diseases. International Publication No. WO 98/26783, published Jun. 25, 1998, discloses the use of certain steroid compounds with anti-glucocorticoid activity for prevention or treatment of psychoses or addictive behavior. International Publication No. WO 98/27986, published Jul. 2, 1998, discloses methods for treating non-insulin dependent Diabetes Mellitus (NIDDM), or Type II Diabetes, by administering a combination of treatment agents exhibiting glucocorticoid receptor type I agonist activity and glucocorticoid receptor type II antagonist activity. Treatment agents such as certain steroid compounds having both glucocorticoid receptor type I agonist activity and glucocorticoid receptor type II antagonist activity are also disclosed. International Publication No. WO 98/31702, published Jul. 23, 1998, discloses certain 16-hydroxy-11-(substituted phenyl)estra-4,9-diene derivatives reportedly useful in treatment or prophylaxis of glucocorticoid dependent diseases or symptoms. Published European Patent Application 0 903 146, published Mar. 24, 1999, reports that the steroid 21-hydroxy-6,19-oxidoprogesterone (21OH-6OP) is a selective antiglucocorticoid useful for the treatment of diseases associated with an excess of glucocorticoids in the body, such as the Cushing's syndrome or depression. Additional disclosures pertaining to the identification and utility of nuclear hormone receptor agonists and antagonists are provided in U.S. Pat. No. 3,683,091; Japanese Patent Application, Publication No. 45014056, published May 20, 1970; Japanese Patent Application, Publication No. 6-263688, published Sep. 20, 1994; International Publication No. WO 95/10266, published Apr. 20, 1995; Japanese Patent Application, Publication No. 45-36500, published Nov. 20, 1970; European Patent Application, Publication No. 0 188 396, published Jul. 23, 1986; Japanese Patent 09052899, dated Feb. 25, 1997; and U.S. Pat. No. 5,696,127. All of the above cited patents and publications are incorporated herein by reference.

Although there are compositions and methods proposed in the art for modulating nuclear hormone receptor activity and thereby ameliorating disease mediated directly or indirectly by the action of nuclear hormone receptors, there is a continuing need for and a continuing search in the field for additional and more effective compositions and methods to satisfy these objectives. Thus, the identification of compounds and methods that effectively modulate nuclear hormone receptor activity with minimal side effects remains an important objective in the art. As disclosed herein, bacterial products can be used in methods to identify compounds that modulate nuclear hormone receptor activity. In one specific, non-limiting example, the bacterial product is from anthrax.

### Anthrax

Anthrax is a zoonotic illness that has been recognized for a long period of time. In the 1870s, Robert Koch demonstrated for the first time the bacterial origin of a specific disease, with his studies on experimental anthrax, and also discovered the spore stage that allows persistence of the organism in the environment. Shortly afterward, Bacillus anthracis was recognized as the cause of woolsorter disease (inhalational anthrax). Bacillus anthracis is a large, gram-positive, sporulating rod, with square or concave ends.

Human cases of anthrax are invariably zoonotic in origin, with no convincing data to suggest that human-to-human transmission has ever taken place. Primary disease takes one of three forms: (1) Cutaneous, the most common, results from contact with an infected animal or animal products; (2) Inhalational is much less common and a result of spore deposition in the lungs, while (3) Gastrointestinal is due to ingestion of infected meat. Most literature cites cutaneous disease as constituting the large majority (up to 95%) of anthrax cases.

Anthrax disease occurs when spores enter the body, germinate to the bacillary form, and multiply. In cutaneous disease, spores gain entry through cuts, abrasions, or in some cases through certain species of biting flies. Germination is thought to take place in macrophages, and toxin release results in edema and tissue necrosis but little or no purulence, probably because of inhibitory effects of the toxins on leukocytes. Generally, cutaneous disease remains localized, although if untreated it may become systemic in up to 20% of cases, with dissemination via the lymphatics. In the gastrointestinal form, B. anthracis is ingested in spore-contaminated meat, and may invade anywhere in the gastrointestinal tract. Transport to mesenteric or other regional lymph nodes and replication occur, resulting in dissemination, bacteremia, and a high mortality rate. As in other forms of anthrax, involved nodes show an impressive degree of hemorrhage and necrosis.

The pathogenesis of inhalational anthrax is more fully studied and understood. Inhaled spores are ingested by pulmonary macrophages and carried to hilar and mediastinal lymph nodes, where they germinate and multiply, elaborating toxins and overwhelming the clearance ability of the regional nodes. Bacteremia occurs, and death soon follows. Penicillin remains the drug of choice for treatment of susceptible strains of anthrax, with ciprofloxacin and doxycycline employed as suitable alternatives. Some data in experimental models of infection suggest that the addition of streptomycin to penicillin may also be helpful. Penicillin resistance remains extremely rare in naturally occurring strains, however the possibility of resistance should be suspected in a biological warfare attack. More severe forms of anthrax require intensive supportive care and have a high mortality rate despite optimal therapy. The use of anti-anthrax serum, while no longer available for human use except in the former Soviet Union, was thought to be of some use in the preantibiotic era, although no controlled studies were performed.

Death from anthrax is reported to result from systemic shock resembling LPS-induced toxic shock (P. Hanna, J. Appl. Microbiol. 87:285, 1999; P. C. Hanna et al., Trends Microbiol. 7:180, 1999), although the role of inflammatory cytokines in this process has been questioned (J. L. Erwin et al., Infect. Immun., 69:1175, 2001).

A “bacterial product” is a compound produced by a bacteria, such as a protein, superantigen, toxin or a polysaccharide. An exemplary bacterial product is a bacterial wall protein, soluble bacterial protein, or lipopolysaccharide The virulence of B. anthracis is dependent on two bacterial products, both of which are toxins, lethal factor (LF) and edema, as well as on the bacterial capsule. The importance of a toxin in anthrax pathogenesis was demonstrated in the early 1950s, when sterile plasma from anthrax-infected guinea pigs caused disease when injected into other animals (Smith et al., Nature, 173:869-870, 1954). It has since been shown that the anthrax toxins are composed of three entities, which in concert lead to some of the clinical effects of anthrax (Stanley et al., J. Gen. Microbiol., 26:49-66, 1961; Beall et al., J. Bacteriol., 83:1274-1280, 1962). The first of these, protective antigen (PA), is an 83 kD protein so named because it is the main protective constituent of anthrax vaccines. PA binds to the anthrax toxin receptor (ATR) on target cells and is then proteolytically cleaved by the enzyme furin of a 20 kd fragment (K. A. Bradley et al., Nature, 414:225, 2001; K. R. Klimpel et al., Proc. Natl. Acad. Sci. U.S.A., 89:10277, 1992).

The smaller cleaved 63 kD PA remnant (PA₆₃) oligomerizes features a newly exposed, second binding domain and binds to either EF, an 89 kD protein, to form edema toxin, or LF, a 90 kD protein, to form lethal toxin (LeTx) (Leppla et al., Salisbury Med. Bull. Suppl., 68:4143, 1990), and the complex is internalized into the cell by (Y. Singh et al., Infect. Immun. 67:1853, 1999; A. M. Friedlander, J. Biol. Chem, 261:7123, 1986). From these endosomes, the PA₆₃ channel enables translocation of LF and EF to the cytosol by a pH- and voltage-dependant mechanism (J. Zhao et al., J. Biol. Chem., 270:18626, 1995; J. Wesche et al., Biochemistry, 37:15737, 1998; R. O. Blaustein et al., Proc. Natl. Acad. Sci. U.S.A., 86:2209, 1989).

Edema factor, a calmodulin-dependent adenylate cyclase, acts by converting adenosine triphosphate to cyclic adenosine monophosphate. Intracellular cyclic adenosine monophosphate levels are thereby increased, leading to the edema characteristic of the disease (Leppla et al., Proc. Natl. Acad. Sci. USA, 79:3162-3166, 1982).

It is the lethal toxin produced by Bacillus anthracis that causes the death of infected hosts (C. Pezard et al., Infect. Immun., 59:3472, 1991). Lethal toxin has been demonstrated to lyse macrophages at high concentration, while inducing the release of tumor necrosis factor and interleukin 1 at lower concentrations (Hanna et al., Proc. Natl. Acad. Sci. USA, 90:10198-10201, 1993; Freidlander J. Biol. Chem., 261:7123-7126, 1986). It has been shown that a combination of antibodies to interleukin 1 and tumor necrosis factor was protective against a lethal challenge of anthrax toxin in mice, as was the human interleukin 1 receptor antagonist (Hanna et al., supra). Macrophage-depleted mice were shown to resist lethal toxin challenge, but to succumb when macrophages were reconstituted. The genes for both the toxin and the capsule are carried by plasmids, designated pX01 and pX02, respectively.

Although anthrax vaccination dates to the early studies of Greenfield and Pasteur, the “modern” era of anthrax vaccine development began with a toxin-producing, unencapsulated (attenuated) strain in the 1930s. Administered to livestock as a single dose with a yearly booster, the vaccine was highly immunogenic and well tolerated in most species, although somewhat virulent in certain species. This preparation is essentially the same as that administered to livestock around the world today.

The first human anthrax vaccine was developed in the 1940s from nonencapsulated strains. This live spore vaccine is administered by scarification with a yearly booster. Studies show a reduced risk of 5- to 15-fold in occupationally exposed workers (Shlyakhov et al., Vaccine, 12:727-730, 1994). British and U.S. vaccines were developed in the 1950s and early 1960s, with the U.S. product an aluminum hydroxide-adsorbed, cell-free culture filtrate of an unencapsulated strain (V770-NP1-R), and the British vaccine an alum-precipitated, cell-free filtrate of a Sterne strain culture. The U.S. vaccine has been shown to induce high levels of antibody only to protective antigen, while the British vaccine induces lower levels of antibody to protective antigen but measurable antibodies against lethal factor and edema factor (Turnbull et al., Infect. Immunol. 52:356-363, 1986; Turnbull et al., Med. Microbiol. Immunol. 177:293-303, 1988). Neither vaccine has been examined in a human clinical efficacy trial. A high number of the recipients of the vaccine have reported some type of reaction to vaccination. Manufacturer labeling for the current Michigan Department of Public Health anthrax vaccine adsorbed (AVA) product cites a 30% rate of mild local reactions and a 4% rate of moderate local reactions with a second dose.

One significant limitation on the use of vaccines is that existing vaccines provide no protection against a number of strains of B. anthracis. Recent incidents, such as the suspected use of biological and chemical weapons during the Persian Gulf War, underscore the threat of biological warfare either on the battlefield or by terrorists. Anthrax has been the focus of much attention as a potential biological warfare agent for at least six decades, and modeling studies have shown the potential for use in an offensive capacity. Dispersal experiments with the simulant Bacillus globigii in the New York subway system in the 1960s suggested that release of a similar amount of B. anthracis during rush hour would result in 10,000 deaths. On a larger scale, the World Health Organization estimated that 50 kg of B. anthracis released upwind of a population center of 500,000 would result in up to 95,000 fatalities, with an additional 125,000 persons incapacitated (Huxsoll et al., JAMA, 262:677-679, 1989). Both on the battlefield and in a terrorist strike, B. anthracis has the attribute of being potentially undetectable until large numbers of seriously ill individuals present with characteristic signs and symptoms of inhalational anthrax. Given these findings, efforts to prevent the disease or to ameliorate or treat its effects are of major importance.

### Modulation of Activity of a Nuclear Hormone Receptor by a Bacterial Product

Compounds, pharmaceutical compositions, and methods for modulating processes mediated by a nuclear hormone receptor are disclosed herein. In several embodiments, the nuclear hormone receptor is a glucocorticoid receptor (GR), androgen receptor (AR), mineralocorticoid receptor (MR), progestin receptor (PR), estrogen receptor (ER), thyroid hormone receptor (TR), vitamin D receptor (VDR), retinoid receptor (RAR or RXR), peroxisome receptor (XPAR or PPAR), or icosanoid receptor (IRs). In other specific embodiments the receptor is an orphan receptor, for example a steroid receptor and/or thyroid receptor.

It is disclosed herein that a bacterial product, specifically a bacterial toxin affects the activity of the glucocorticoid receptor (GR). In one example, the toxin is the anthrax lethal factor (LF) produced by Bacillus anthracis. In one example, this activity of the exemplary bacterial toxin LF can be assayed using a reporter system. A specific non-limiting example of a reporter system is a transient gluccocorticoid responsive element (GRE)-luciferase transfection system, which establishes GR repression by LF to a level of 50%, and at very low concentrations as low as 1.5-2.0 ng/ml. In cellular systems when LF is exogenously applied this effect occurs only in the presence of the anthrax protective antigen (PA), a protein produced by the anthrax bacteria that is essential for transport of LF into cells. However, in certain embodiments, LF alone may mediate GR repression and related effects when delivered internally in cells (such as when cells have been transduced with a polynucleotide encoding LF to express the protein endogenously).

Bacterial products of use include bacterial wall proteins and other products (such as streptococcal or staphylococcal cell walls and lipopolysaccharide (LPS), and soluble antigens of bacteria. The products of interest can exert various effects on infected hosts, for example by causing damage to cell membranes, inhibition of protein synthesis, activation of second messenger pathways, activation of immune responses, and/or degradation of host proteins (such as by functioning as a metalloprotease). In specific embodiments, the bacterial product is a bacterial toxin. As used herein bacterial toxins include bacterial products that mediate toxic effects, inflammatory responses, stress, shock, chronic sequelae, or mortality in a susceptible host. Exemplary bacterial toxins are anthrax LF and LeTx, and metalloenzymes of Clostridium tetanus and C. botulinum bacteria. In one embodiment, the bacterial product is not endotoxin. In other embodiments, the bacterial product is a bacterial antigen, for example a pyrogenic toxin superantigen (PTSAg) (such as a staphylococcal enterotoxin, exfoliative toxin, or toxic-shock toxin. In other specific embodiment, the bacterial product is a toxin, but is not endotoxin. Table 1, below, sets forth an exemplary list of candidate bacterial products characterized generically as bacterial toxins that will find use within the methods and compositions disclosed herein (see also review by Schmidtt et al., Emerg. Infect. Dis., 5:224-234, 1999).

LF and other bacterial products can be selected for use by their ability to interact with one or more nuclear hormone receptors, directly or indirectly (such as by interacting with a co-factor of a nuclear hormone receptor), in a manner that modulates activity of the receptor(s). Often, receptor modulation in this context will suppress or amplify an inflammatory response, autoimmune symptom, or other adverse symptoms in the subject, for example by repressing the anti-inflammatory effects of the glucocorticoids. In the case of anthrax, the inflammatory, toxic and/or lethal effects of Bacillus anthracis may be caused at least in part by antagonism/repression of the glucocorticoid receptor by bacterial products. In one emobidment, this interaction is not through the ligand binding domain.

As discussed briefly above, anthrax toxins are composed of three proteins: lethal factor (LF), protective antigen (PA) and edema factor (EF) (S. H. Leppla, Comprehensive Sourcebook of Bacterial Protein Toxins. ed., 243-63, 1999; S. H. Leppla Bacterial Protein Toxins. ed., 445-72, 2000). PA facilitates entry of LF and EF into cells. LF is a 90 kD metalloprotease, for which the crystal structure has recently been determined (A. D. Pannifer et al., Nature, 414:229-33, 2001). All three genes are encoded by the plasmid pXO1 (M. Mock et al., Annu. Rev. Microbiol., 55:647-71, 2001). Together, LF and PA constitute the lethal toxin (LeTx), and EF and PA the edema toxin.

Studies directed at the mechanism of action of LeTx have mainly focused on its action in cleaving MAPKK. While this action effectively and rapidly removes this important signal transduction molecule, evidence of some transient activation of the system, such as phosphorylation of ERK, has also been observed (R. Pellizzari et al.,. Int. J. Med. Microbiol., 290:421-7, 2000). Nonetheless, LF resistant and susceptible cell lines show equal MAPKK proteolysis by LeTx (R. Pellizzari et al., Int. J. Med. Microbiol., 290:421-7, 2000; P Pellizzari et al., FEBS letters. 462:199-204, 1999). Thus, while LeTx does cleave MAPKK, other or additional biological activities could be needed to cause its toxic and lethal effects. Such an activity, namely the interaction with a nuclear receptor, is demonstrated herein.

### Synopses

TABLE 1
Characteristics of bacterial toxins^{a}

| | | | | Toxin |
|---|---|---|---|---|
| | | | | implicated |
| Organism/Toxin | Mode of Action | Target | Disease | in disease [symbol] |
| Damage membranes | | | | |
| Aeromonas hydrophila/aerolysin | Pore-former | Glycophorin | Diarrhea | (yes) |
| Clostridium perfringens/ | Pore-former | Cholesterol | Gas gangrene^{c} | ? |
| perfringolysin O | | | | |
| Escherichia coli/hemolysin^{d} | Pore-former | Plasma membrane | UTIs | (yes) |
| Listeria monocytogenes/ | Pore-former | Cholesterol | Foodborne | (yes) |
| listeriolysin O | | | systemic illness, | |
| | | | meningitis | |
| Staphyloccocus aureus/a-toxin | Pore-former | Plasma membrane | Abcesses^{c} | (yes) |
| Streptococcus | Pore-former | Cholesterol | Pneumonia^{c} | (yes) |
| pneumoniael/pneumolysin | | | | |
| Streptococcus | Pore-former | Cholesterol | Strep throat, Sf^{c} | ? |
| pyogenesl/streptolysin O | | | | |
| Inhibit protein synthesis | | | | |
| Corynebacterium | ADP-ribosyltransferase | Elongation factor 2 | Diphtheria | yes |
| diphtheriae/diphtheria toxin | | | | |
| E. coli/Shigella dysenteriae/ | N-glycosidase | 285 rRNA | HC and HUS | yes |
| Shiga toxins | | | | |
| Pseudomonas aeruginosa/ | ADP-ribosyltransferase | Elongation factor 2 | Pneumonia^{c} | (yes) |
| exotoxin A | | | | |
| Activate second messenger | | | | |
| pathways | | | | |
| E. coli | | | | |
| CNF | Deamidase | Rho G-proteins | UTIs | ? |
| LT | ADP-ribosyltransferase | G-proteins | Diarrhea | yes |
| ST^{d} | Stimulates guanylate | guanylate cyclase | Diarrhea | yes |
| | cyclase | receptor | | |
| CLDT^{d} | G2 block | Unknown | Diarrhea | (yes) |
| EAST | ST-like? | Unknown | Diarrhea | ? |
| Bacillus anthracis/edema factor | Adenylate cyclase | ATP | Anthrax | yes |
| Bordetella pertussis/ | Deamidase | Rho G-proteins | Rhinitis | (yes) |
| dermonecrotic toxin | | | | |
| pertussis toxin | ADP-ribosyltransferase | G-protein(s) | Pertussis | yes |
| Clostridium botulinum/C2 toxin | ADP-ribosyltransferase | Monomeric G-actin | Botulism | ? |
| C. botulinum/C3 toxin | ADP-ribosyltransferase | Rho G-protein | Botulism | ? |
| Clostridium difficilel | | | | |
| toxin A | Glucosyltransferase | Rho G-protein(s) | Diarrhea/PC | (yes) |
| toxin B | Glucosyltransferase | Rho G-protein(s) | Diarrhea/PC | ? |
| Vibrio cholerae/cholera toxin | ADP-ribosyltransferase | G-protein(s) | Cholera | yes |
| Activate immune response | | | | |
| S. aureus/ | Superantigen | TCR and MHC B | Food poisoning^{c} | yes |
| enterotoxins | | | | |
| exfoliative toxins | Superantigen (and serine | TCR and MHC II | SSS^{c} | yes |
| | protease?) | | | |
| toxic-shock toxin | Superantigen | TCR and MHC II | TSS^{c} | yes |
| S. pyogerces/pyrogenic | Superantigens | TCR and MHC II | SF/TSS^{c} | yes |
| exotoxins | | | | |
| Protease | | | | |
| B. anthracis/lethal factor | Metalloprotease | MAPKK1/MAPKK2 | Anthrax | yes |
| C. botulinum/neurotoxinsA-G | Zinc-metalloprotease | VAMP/ | Botulism | yes |
| | | synaptobrevin, | | |
| | | SNAP-25, syntaxin | | |
| Clostridium tetani/tetanus toxin | Zinc-metalloprotease | VAMP/synaptobrevin | Tetanus | yes |
| ^{a}Abbreviations: CNF, cytotoxic necrotizing factor; LT, heat-labile toxin; ST, heat-stable toxin; CLDT, cytolethal distending toxin; EAST, enteroaggregative E. coli heat-stable toxin; TCR, T-cell receptor; MHC II, major histocompatibility complex class II; MAPKK, mitogen-activated protein kinase kinase; VAMP, vesicle-associated membrane protein; SNAP-25, synaptosomal associated protein; UTI, urinary tract infection; HC, hemorrhagic colitis; HUS, hemolytic uremic syndrome; PC, | | | | |
| # antibiotic associated pseudomembranous colitis; SSS, scalded skin syndrome; SF, scarlet fever; TSS, toxic-shock syndrome. | | | | |

^{b}Yes strong causal relationship between toxin and disease; (yes), role in pathogenesis has been shown in animal model or appropriate cell culture; ?, unknown.
^{c}Other diseases are also associated with the organism.
^{d}Toxin is also produced by other genera of bacteria.

Radioligand competition studies detailed below indicate that neither LF nor PA, alone and/or together, competes with dexamethasone for binding to the ligand binding site of GR, nor do they interfere with GR-GRE DNA binding in electrophoretic mobility shift assays (EMSAs). Thus, anthrax lethal toxin (LeTx) and lethal factor (LF) specifically represses activation of glucocorticoid receptor in a dose-related, non-competitive, non-ligand or DNA-binding manner. This bacterial product can exert its effect on nuclear hormone receptor repression through a cofactor involved in the interaction between nuclear hormone receptors and the basal transcription machinery, and/or by acting itself as a co-repressor. In one embodiment, the repression of GR is mediated through the DNA binding domain (DBD), co-factor binding, or downstream pathways that interact with these domains of the receptor and the basal transcription machinery.

In another embodiment, repression of a nuclear hormone receptor by a bacterial product is not mediated by inhibition of a MEK1 or MAPKK pathway. This finding contrasts with many reports suggesting that the toxic or shock-related activities of LF are mediated by LF's proposed metalloprotease function and a putative degradation by LF of proteins involved in the MEK1 and/or MAPKK pathway(s). The MAPK pathway consists of three separate pathways, MEK, SEK and p38 (Pellozzari et al., FEBS Lett., 462:199, 1999; Pellozzari et al., J. Med. Microbiol. 290:421, 2000).

The MEK and p38 pathways are known to be targets of LeTx. In the examples below it is demonstrated that PD98059, an inhibitor of the MEK pathway, does not have any GR specific effect in a transient transfection system. SB203580, an inhibitor of the p38 pathway also has no effect on GR-mediated transactivation in a GRE-luciferase system. LeTx is known to degrade some proteins of the MAPK pathway including MEK. The data presented herein indicate that MEK degradation alone does not determine LeTx sensitivity and that other factor(s) must be involved. As disclosed herein, repression of GR and other nuclear hormone receptor hormones is a factor in determining LeTx sensitivity.

In yet another embodiment, repression of a nuclear hormone receptor by a bacterial product is not mediated by a change in the number of nuclear hormone receptors on a cell. Thus, cells treated with the bacterial product have substantially the same number of nuclear hormone receptors as cells not treated with the bacterial product. In several example, the number of receptors does not change by more than about 1%, more than about 5%, more than about 10% or more than 25% upon treatment with the bacterial product (as compared to cells not treated with the bacterial product). In other examples, no statistically significant difference in the number of receptors is observed following treatment with the bacterial product. One of skill in the art can readily identify appropriate statistical analyses for this determination. Exemplary methods for determining the number of nuclear hormone receptors on cells are provided in the Examples section below.

LF activity is specific for some nuclear hormone receptors, whereas other bacterial products as disclosed herein will be specific for the same or different receptors, or more generalized by acting to repress a wider group of nuclear hormone receptors. In the case of anthrax LF and LeTx, this bacterial toxin is demonstrated herein to repress GR (Type 1 GR) but not the mineralocorticoid (MR, Type II GR) receptor, and to repress estrogen receptor-α (Er-α) but not ER-β, and the progesterone receptor B (PR-B).

Nuclear hormone receptor agonists and antagonists are useful to influence basic, life sustaining systems of the body, including carbohydrate, protein and lipid metabolism, electrolyte and water balance, and the functions of the cardiovascular, kidney, central nervous, immune, skeletal muscle and other organ and tissue systems. In this regard, GR and MR modulators have proved useful in the treatment of inflammation, tissue rejection, auto-immunity, hypertension, various malignancies, such as luekemias, lymphomas, and thyroid, breast and prostate cancers, Cushing's syndrome, glaucoma, obesity, rheumatoid arthritis, acute adrenal insufficiency, congenital adrenal hyperplasia, osteoarthritis, rheumatic fever, polymyositis, polyarteritis nodosa, granulomatous polyarteritis, allergic diseases such as urticaria, drug reactions and hay fever, asthma, a variety of skin diseases, inflammatory bowel disease, hepatitis and cirrhosis. Accordingly, in exemplary embodiments, GR and MR modulatory compounds are useful as immuno stimulants and repressors, wound healing and/or tissue repair agents, catabolic/antianabolic activators, and as antibacterial or anti-viral agents (such as for treatment or prevention of symptoms related to anthrax, herpes simplex viral infection and related symptoms). Additional diseases that may prove amenable to diagnosis and/or management using the methods and compositions disclosed herein include, but are not limited to, Parkinson's disease, cardiovascular disease including restenosis, anxiety, depression, psychosis, various viral infections, including HIV and HSV, proliferative and hyperproliferative disorders, including restenosis and psoriasis.

Autoimmune diseases or disorders that can be treated, prevented, and/or diagnosed by polynucleotides, polypeptides, antibodies, and/or agonists or antagonists of nuclear receptors include but are not limited to, one or more of the following: systemic lupus erythematosus, rheumatoid arthritis, ankylosing spondylitis, multiple sclerosis, autoimmune thyroiditis, Hashimoto's thyroiditis, autoimmune hemolytic anemia, hemolytic anemia, thrombocytopenia, autoimmnune thrombocytopeni purpura, autoimmune neonatal thrombocytopenia, idiopathic thrombocytopenia purpura, purpura a (such as Henloch-Scoenlein purpura), autoimmunocytopenia, Goodpasture's syndrome, Pemphigus vulgaris, myasthenia gravis, Grave's disease (hyperthyroidism), and insulin-resistant diabetes mellitus.

Additional disorders that are likely to have an autoimmune component that can be treated, prevented, and/or diagnosed using the methods and compositions disclosed herein include, but are no limited to, type II collagen-induced arthritis, antiphospholipid syndrome, dermatitis, allergic encephalomyelitis, myocarditis, relapsing polychondritis, rheumatic heart disease, Neuritis, Uveitis Ophthalmia, Polyendocrinopathies, Reiter's Disease, Stiff-Man Syndrome, Autoimmune Pulmonary Inflammation, Autism, Guillain-Barre Syndrome, insulin dependent diabetes mellitis, and autoimmune inflammatory eye.

Yet additional disorders that are likely to have an autoimmune component that can be treated, prevented, and/or diagnosed with the compositions disclosed herein include, but are not limited to, scleroderma with anti-collagen antibodies (often characterized, such as by nucleolar and other nuclear antibodies), mixed connective tissue disease (often characterized, such as by antibodies to extractable nuclear antigens (for example, ribonucleoprotein)), polymyositis (often characterized, for example, by nonhistone ANA), pernicious anemia (often characterized, for example, by antiparietal cell, microsomes, and intrinsic factor antibodies), idiopathic Addison's disease (often characterized, for example, by humoral and cell-mediated adrenal cytotoxicity, infertility (often characterized, for example, by antispermatozoal antibodies), glomerulonephritis (often characterized, for example, by glomerular basement membrane antibodies or immune complexes), bullous pemphigoid (often characterized, for example, by IgG and complement in basement membrane), Sjogren's syndrome (often characterized, for example, by multiple tissue antibodies, and/or a specific nonhistone ANA (SS-B)), diabetes mellitus (often characterized, for example, by cell-mediated and humoral islet cell antibodies), and adrenergic drug resistance (including adrenergic drug resistance with asthma or cystic fibrosis) (often characterized, for example, by beta-adrenergic receptor antibodies).

Additional disorders that may have an autoimmune component that can be treated, prevented, and/or diagnosed with the compositions disclosed herein include, but are not limited to, chronic active hepatitis (often characterized, for example by smooth muscle antibodies), primary biliary cirrhosis (often characterized, for example, by mitchondrial antibodies), other endocrine gland failure (often characterized, for example, by specific tissue antibodies in some cases), vitiligo (often characterized, for example, by melanocyte antibodies), vasculitis (often characterized, for example, by Ig and complement in vessel walls and/or low serum complement), post-MI (often characterized, for example, by myocardial antibodies), cardiotomy syndrome (often characterized, for example, by myocardial antibodies), urticaria (often characterized, for example, by IgG and IgM antibodies to IgE), atopic dermatitis (often characterized, for example, by IgG and IgM antibodies to IgE), asthma (often characterized, for example, by IgG and IgM antibodies to IgE), and many other inflammatory, granulamatous, degenerative, and atrophic disorders.

For treatment and prevention of bacterial disease and associated inflammatory, autoimmune, toxic (including shock), and chronic and/or lethal sequelae associated with bacterial infection a wide variety of effective compositions and methods are provided. In one embodiment, one or more symptoms or associated effects of exposure to and/or infection with anthrax is/are prevented or treated by administration to a mammalian subject at risk of acquiring or presenting with the symptom(s) of an effective amount of an agent that affects nuclear hormone receptor activity. In exemplary embodiments, these treatment and prophylactic methods and compositions employ drugs and other agents identified according to the methods herein to bypass or diminish blockade of a nuclear hormone receptor mediated by a bacterial product (for example, LF blockade of the glucocorticoid receptor (GR), PR or other nuclear hormone receptor(s)). Alternative approaches to bypassing or reducing nuclear hormone receptor activation and/or repression involve, for example in the case of anthrax, treatment with glucocorticoid or other nuclear hormone receptor agonists or antagonists, or with agents that interact with GR and LF/PA, or with agents that enhance or repress GR and/or other nuclear hormone receptor co-factors.

Therapeutic compositions and methods for prevention or treatment of toxic or lethal effects of bacterial infection are applicable to a wide spectrum of infectious agents. Non-lethal toxicities that will be ameliorated by these methods and compositions include fatigue syndromes, inflammatory/autoimmune syndromes, hypoadrenal syndromes, weakness, cognitive symptoms and memory loss, mood symptoms, neurological and pain syndromes and endocrine symptoms.

These compositions and methods are also applicable to treatment and prevention of toxic effects of exposure to anthrax and/or related bacterial vaccines. Reports indicate that Gulf War syndrome symptoms of fatigue, depression, inflammatory/autoimmune, weakness, memory loss, neurological, pain, endocrine and other symptoms, may be related to vaccination with anthrax vaccine. The currently available anthrax vaccine, derived from a bacterial cell filtrate of Bacillus anthracis contains variable amounts of LF, and acute and chronic effects are probably related to interactions of vaccine components with GR and/or its cofactors and other nuclear hormone receptors and/or their co-factors. Thus, as disclosed herein, antagonists of GR can be used for the prevention and treatment of side effects related to the anthrax vaccine, as well as a means to produce vaccine without or with lower risk of such side effects.

Additional embodiments are directed to diagnostic compositions and methods to identify individuals at risk for toxic effects or long-term deleterious effects of exposure to pathogenic bacteria, for example anthrax bacteria, and their cognate vaccines. Certain strains of rodents show enhanced susceptibility to lethal effects of exposure to anthrax. The disclosure herein implicates differences in characteristics, number or regulation of GR, GR co-factors or other nuclear hormone receptors and their co-factors for these strain differences. Identification and characterization of GR, its co-factors and other nuclear hormone receptor co-factors according to the present disclosure will provide effective tools for identifying individuals who may be genetically or otherwise predisposed to development of toxic lethal or long term chronic effects from exposure to bacterial pathogens and vaccines directed to them. Thus, in one embodiment a bacterial produced can be used to identify a subject having or at risk of developeing a disorder, such as a disorder associated with a cofactor of a nuclear hormone receptor.

In additional aspects, the methods and compositions disclosed herein are useful for identification of environmental agents, including other bacterial products (for example, products of food-borne pathogens) that mediate idiopathic inflammatory, autoimmune, fatigue, memory loss, endocrine and other syndromes. Certain individuals exposed to small amounts of bacterial products, such as those derived from anthrax, presenting certain genetic or physiological backgrounds, are predisposed to development of chronic syndromes, including fatigue syndromes, inflammatory/autoimmune syndromes, hypoadrenal syndromes, weakness, cognitive symptoms and memory loss, mood symptoms, neurological and pain syndromes and endocrine symptoms. In this context, the methods and compositions disclosed herein employed to detect, and alternatively to treat and/or prevent, such ubiquitous environmental exposures and associated symptoms. For example, methods for screening for LF/PA-like bacterial products or other environmental agents that interact with nuclear hormone receptors or their co-factors in a manner associated with disease or other adverse symptoms or conditions in mammalian subjects.

In one embodiment, LF and other bacterial products that specifically block, degrade or otherwise interact with one of the GR or other nuclear hormone receptor co-factors are employed as reagents in various screening methods to identify, for example whether a specific co-factor is involved in an important hormonal action. Certain screening methods will “knock out” a subject cofactor (for example, in an engineered cell or knock-out mouse) in order to clarify the role of the co-factor in mediating receptor modulation and/or disease. As such, a large panel of known cofactors of nuclear hormone receptors will find utility and are therefore incorporated within various embodiments of the compositions and/or methods thereof. Exemplary members of this panel are set forth below in Table 2.

TABLE 2
CO-ACTIVATORS OF NUCLEAR HORMONE RECEPTORS

| Name | Other names |
|---|---|
| p160 family | |
| ERAP160 | RIP160 |
| SRC-1 | N-CoA1 |
| TIF2 | GRIP1, SRC-2 |
| p/CIP | ACTR, RAC3, AIB-1, TRAM-1, |
| | SRC-3 |
| p140 family | |
| p140 | ERAP140, RIP140 |
| p300 family | |
| p300 | |
| CBP | |
| P/CAF (ADA/SAGA) complex | |
| PCAF | |
| GCN5 | |
| Tra1/TRRAP | |
| PAF65α | |
| PAF65β | |
| TAF31 | hTAF_{II}31 |
| TAF30 | hTAF_{II}30 |
| TAF20 | hTAF_{II}20 |
| hAda2 | |
| hSPT3 | |
| hAda3 | |
| hTAF_{II}15 | |
| Basal Transcriptional machinery | |
| TFIID | |
| TFIIH | |
| TFIIE | |
| TEIIF | |
| TBP | |
| TBP-associated factors (TAFs) | |
| TAF250 | |
| TAF130 | |
| TAF28 | |
| TAF18 | |
| TAF55 | |
| TAF150 | |
| TAF70 | |
| TAF31 | |
| TAF20 | |
| TAF100 | |
| TAF30 | |
| Universal stimulatory activity (USA) | |
| NC2 - subunits Dr1 and Drap1 | |
| PC2 | |
| PC4 | |
| Mediator/SRB | |
| Med10 | |
| Med7 | |
| Med6 | |
| Med1 | |
| Med2 | |
| Med3 | |
| Med4 | |
| Med5 | |
| Gal11 | |
| Sin4 | |
| Rgr1 | |
| Rox3 | |
| NAT complex | |
| SRB10/CDK8 | |
| Srb7 | |
| Srb10 | |
| Rgr1 | |
| Med6 | |
| P230 | |
| P150 | |
| P140 | HSur2 |
| P95 | |
| P90 | |
| P70 | |
| P56 | Cdk8 |
| P45 | |
| P37 | |
| P36p33 | |
| P31 | Cyclin C |
| P30 | |
| P23 | |
| P22 | |
| P21 | |
| P17 | |
| P14 | |
| SMCC complex | |
| SRB11/cyclinC | |
| CRSP complex | |
| Med7 | |
| Rgr1 | |
| CRSP200 | |
| CRSP150 | |
| CRSP130 | |
| CRSP77 | |
| CRSP70 | |
| CRSP34 | |
| CRSP33 | |
| Drip complex | |
| DRIP205 | |
| DRIP240 | |
| DRIP250 | |
| DRIP70 | |
| DRIP77 | |
| DRIP92 | |
| DRIP100 | |
| DRIP130 | |
| DRIP150 | |
| DRIP97 | |
| DRIP70-2 | |
| Cdk8 | |
| DRIP36 | |
| DRIP34 | |
| DRIP33 | |
| hSrb7 | |
| hMed10 | |
| TRAP complex | |
| TRAP80 | |
| TRAP93 | |
| TRAP95 | |
| TRAP97 | |
| TRAP100 | |
| TRAP150 | |
| TRAP170 | |
| TRAP220 | |
| TRAP230 | |
| TRAP240 | |
| hSrb10 | |
| hMed7 | |
| hMed6 | |
| hTRF | |
| hSrb11 | |
| hSoh1 | |
| hSrb7 | |
| hNut2 | |
| ARC complex | |
| ARC250 | |
| ARC240 | |
| ARC205 | |
| ARC150 | |
| ARC130 | |
| ARC105 | TIG-1 |
| ARC100 | |
| ARC92 | |
| ARC77 | |
| ARC70 | |
| ARC42 | |
| ARC36 | |
| ARC34 | |
| ARC33 | |
| ARC32 | |
| NUA3 COMPLEX (YEAST) | |
| NuA4 complex (Yeast) | |
| E3 Ubiquitin-protein ligases | |
| RPF-1 | |
| E6-AP | |
| ARNIP | |
| Histone Methyltransferases | |
| CARM-1 | |
| PRMT-1 | |
| Suv39H1 | |
| G9a | |
| Set 9 | Set 7 |
| Chromatin modifying ATPases | |
| SWI/SNF COMPLEX | |
| Brahma (BRM) | |
| Brahma-related gene-1 (BRG-1) | hSNF2□ |
| BRG-1 associated factors (BAFs) | |
| INI1 | BAF47 |
| BAF155 | |
| BAF170 | |
| BAF57 | |
| BAF250 | |
| hSNF5 | INI1, BAF47 |
| BAF60 | |
| BAF53 | |
| NURF complex | |
| NURF301 | |
| NURF140 | |
| NURF55 | |
| NURF38 | |
| NURF215 | |
| NURD complex | |
| Mi-2β | CHD4 |
| Mi-2α | CHD3 |
| HDAC1 | NURD63 |
| HDAC2 | NURD59 |
| RbAp48 | NURD56 |
| RbAp46 | NURD55 |
| MTA1/2 | NURD70 |
| MBD3 | |
| ACF complex | |
| Acf1 | |
| ISWI | |
| CHRAC complex | |
| ACF1 | |
| hSNF2H | |
| hSNF2L | |
| RSF complex | |
| hSNF2h | |
| p325 | |
| TIF1 | |
| NSD-1 | |
| Co-repressors | |
| RIP13 | NCoR |
| SMRT | |
| Sin3-A | Rpd1 |
| Sin3-B | |
| HDAC-1 | Rpd3 |
| HDAC-2 | |
| HDAC-4 | |
| HDAC-5 | |
| HDAC-6 | |
| HDAC-7 | |
| HDAC-3 | |
| RbAp46/48 | |
| Mi-2 | CHD4 |
| MBD2 | |
| MeCP1 | |
| Others | |
| Sug1 | Trip1 |
| GRIP95 | |
| GRIP120 | |
| GRIP170 | |
| ARA₇₀ | |
| RIP80 | |
| CREB | |

See also, Glass et al., Curr. Opin. Cell Biol 9: 222-232, 1997; McKenna et al., Eudocrinology, 143: 2461-2465, 2002.

In other embodiments, LF or other bacterial products that specifically block, degrade or otherwise interact with one of the GR or other nuclear hormone receptor co-factors, antagonists or agonists are employed to induce, amplify or increase or decrease expression of a particular co-factor with which the subject bacterial product interacts, for example in a method or composition to treat or prevent toxicity mediated by LF or another bacterial toxin. Within more detailed aspects, analogs or variants of LF and other bacterial products, and mimetics and drugs that mimic one or more activities (for example, co-factor binding, co-factor degradation, hormone repression) of LF or another bacterial product, may be generated (such as by genetic engineering or chemical modification) to render the product non-toxic while retaining some or all of its function in altering nuclear hormone receptor activity (such as to treat or prevent disease associated with elevated expression or activation of a nuclear hormone receptor). Alternatively, analogs and variants of bacterial products, as well as mimetics and drugs may be developed by routine methods and identified using screening methods presented herein, that block the binding or activity of a corresponding wild type bacterial product to thereby function, directly or indirectly, as an effective nuclear hormone receptor agonist. Such variants and drugs based on LF or other bacterial toxins will often specifically block, degrade, stimulate or otherwise interact with one of the GR or other nuclear hormone receptor co-factors (co-activators or co-repressors), and thereby reduce or enhance the activity of the nuclear hormone receptor. These effects may mediate modulation of activity of one, or a plurality of, nuclear hormone receptors with which these co-factors interact. Thus, novel tools and methods are provided that utilize a limited assemblage of ligand binding agents for blocking or enhancing activity of nuclear hormone receptors. The compositions and methods disclosed herein further provide means to specifically and partially reduce some but not all actions of nuclear hormone receptor hormones, for example when certain target co-factors are specifically expressed in certain tissues but not in others.

In yet additional embodiments, a recombinantly or chemically modified analog, fragment or derivative of LF, or of another bacterial product described herein, is employed in a vaccine or therapeutic formulation or method. Often, the modified analog, fragment or derivative will exhibit substantially reduced or enhanced activity as a modulator (such as a repressor or activator) of nuclear hormone receptor activity compared to a native or wild-type counterpart bacterial product. For example, a modified anthrax LF analog or fragment will exhibit a reduction or increase in a level of GR repression or PR repression in an in vitro or in vivo assay of approximately 20%, 30%, 50%, 75% and up to 95% or greater compared a control level of repression mediated by a native LF protein (alone or complexed with PA). Other analogs and variants of LF or other selected bacterial products will alternatively or additionally specifically inhibit or block, or enhance, interactions of the corresponding native bacterial product with a nuclear hormone receptor. For example, various analogs or variants of LF may competitively inhibit native LF activity (such as cofactor binding, cofactor degradation, and/or GR or PR repression activity) or act as an LF agonist or mimetic in an in vitro or in vivo assay.

The various analogs, variants, derivatives and mimetics of bacterial products provided herein are useful for, inter alia, treatment and/or prevention of diseases, symptoms and conditions relating to bacterial infection, inflammatory responses, and/or autoimmune disorders. In other embodiments, analogs, variants, derivatives and mimetics of bacterial products are useful to provide more effective vaccine compositions and methods, particularly to minimize adverse side effects that attend vaccination using a native or wild-type bacterial product. In one exemplary embodiment, a mutant variant, truncated fragment, or chemically modified derivative of a LF protein is employed as a therapeutic or vaccine agent. The LF variant, fragment or derivative will have substantially reduced or increased activity for nuclear hormone receptor modulation (for example, GR and/or PR repression). At the same time, the LF variant, fragment or derivative will exhibit substantial activity as an immunogen, and/or will inhibit, block or enhance (directly or indirectly) nuclear hormone receptor modulation by native LF or LeTx.

Analogs, variants, derivatives and mimetics of bacterial products will typically be effective to elicit an immune response in a mammalian subject against a corresponding, native bacterial product, whereby the subject will generate a humoral or cell-mediated immune response against the native product that is effective to prevent or reduce infection or alleviate one or more symptoms associated with infection by a pathogen expressing the native product. For example, analogs, variants, derivatives and mimetics of LF and other bacterial products may be generated (for example, by genetic engineering or chemical modification) to render the LF non-toxic. In certain embodiments, the bacterial product will be produced that exhibit increased or reduced activity of the analog, variant, derivative or mimetic for modulation of one or more nuclear hormone receptors (such as to have substantially reduced GR or PR repression activity). Typically, the analogs, variants, derivatives and mimetics of bacterial products thus produced will retain some or all of the antigenic activity possessed by a corresponding wild-type bacterial product to stimulate an effective host immune response (for example, anti-LF antibody production). Thus, more effective bacterial vaccines and immunization methods are provided that yield sufficient stimulation of an anti-bacterial product (for example, anti-LF) immune response in a subject, attended by diminished adverse side effects associated with nuclear hormone receptor modulation that would attend immunization with the corresponding native bacterial product. Such variants, analogs and mimetics of bacterial products will exhibit substantially reduced or enhanced activity for repression or activation of one or more nuclear hormone receptor(s), and can, alternatively or additionally, specifically inhibit, block, or enhance repression or activation of one or more nuclear hormone receptor(s) by a corresponding native bacterial product. For example, administration of a vaccine formulation that includes a modified anthrax LF analog or fragment will be characterized by a reduction or increase in a level of GR and/or PR repression, or in the occurrence of one or more inflammatory or autoimmune symptoms in the immunized subject, compared to that observed following administration of a vaccine formulation comprising a similar dose of native anthrax LF, of approximately 20%, 30%, 50%, 75% and up to 95% or greater. At the same time, the vaccine formulation will elicit an effective immune response (for example, anti-LF antibody production) that is at least 20%, 30%, 50%, 75% and up to 95% or greater in titer or intensity compared to an immune response stimulated by immunization using a similar dose of the corresponding native bacterial product.

The methods disclosed herein allow the production and selection of analogs, variants, derivatives and mimetics of LF and other bacterial products for generation of improved vaccines and other therapeutic formulations. According to the disclosure herein, these analogs, variants, derivatives and mimetics can be routinely generated, such as by creation of truncated fragments or recombinant variants having one or more targeted amino acid substitutions, insertions or deletions. For each subject bacterial product contemplated herein, available structure-function data will be used to select candidate targets for modification within a native protein. For example, in the case of anthrax LF, it is known that the approximately 90 kD protein plays an important role in enhancing protective immunity. An inducible LF expression system has been developed to generate recombinant LF suitable for human vaccine trials (Singh et al., FEMS Microbiol. Lett. 209:301-5, 2002. Generally known methods can be employed to generate recombinant forms of LF and to evaluate immunogenic and nuclear hormone modulator activities of the recombinant LF proteins for development of improved vaccines. Specific targets for chemical modification and/or mutagenesis are also readily determined in accordance with the present disclosure and by reference to published structure-function data for subject bacterial products. For example, the crystal structure of LF and its complex with the N terminus of MAPKK-2 has recently been published by Pannifer et al. (Nature 414:229-33, 2001). LF comprises four domains: domain I binds the membrane-translocating component of anthrax toxin, the protective antigen (PA); domains II, m and IV together create a long deep groove that holds a 16-residue N-terminal tail of MAPKK-2 before cleavage. Domain II resembles the ADP-ribosylating toxin from Bacillus cereus, but the active site is divergent and serves to augment substrate recognition. Domain m is inserted into domain II, and reportedly features a duplicate structural element of domain II. Domain IV is distantly related to the zinc metalloprotease family, and contains the catalytic centre; it also resembles domain I. In one exemplary embodiment, one or more of these domains, for example, domain IV implicated in metalloprotease function, is deleted or mutated to yield an increase or reduction in GR or PR repression activity accompanied by retention of substantial activity of the mutant LF as a prophylactic or therapeutic immunogen. Additional embodiments utilize fusion proteins, conjugates and other analogs and derivatives of bacterial products as vaccine agents according to the above description (for example, see Milne et al., Mol. Microbiol. 15:661-6, 1995, who describe chimeric proteins composed of a PA recognition domain of LF (LFN; residues 1-255) fused to a heterologous protein segment). The purified fusion proteins retained their functionality of complementing PA to mediate translocation of the fusion protein into cells in the presence of PA, and also retained ability to react with antisera against LF.

Analogs, variants, derivatives and mimetics of bacterial products for use include natural or synthetic, therapeutically or prophylactically active, peptides (comprised of two or more covalently linked amino acids), proteins, peptide or protein fragments, peptide or protein analogs, peptide or protein mimetics, and chemically modified derivatives or salts of active peptides or proteins. Thus, as used herein, the terms “analog” or “mimetic” of a bacterial product will often be intended to embrace all of these active species, for example, peptides and proteins, peptide and protein fragments, peptide and protein analogs, peptide and protein mimetics, peptide and protein fusions and other conjugates, and chemically modified derivatives and salts of active peptides or proteins. Often, the peptides or proteins that will find use in the methods disclosed herein are muteins that are readily obtainable by partial substitution, addition, or deletion of amino acids within a naturally occurring or native (for example, wild-type, naturally occurring mutant, or allelic variant) peptide or protein sequence of a known bacterial product (for example, LF). Additionally, biologically active fragments of native peptides or proteins are included. Such mutant derivatives and fragments will often substantially retain a desired biological activity of the native peptide or proteins. In the case of peptides or proteins having carbohydrate chains, biologically active variants marked by alterations in these carbohydrate species are also included.

The peptides, proteins, analogs and mimetics for use within the methods and compositions disclosed herein are often formulated in a pharmaceutical composition comprising an effective amount of the peptide, protein, analog or mimetic that will modulate activity of one or more nuclear hormone receptors or alleviate one or more symptoms of a bacterial infection, inflammatory disorder or autoimmune condition.

In additional embodiments, peptides or proteins for use can be modified by addition or conjugation of a synthetic polymer, such as polyethylene glycol, a natural polymer, such as hyaluronic acid, or an optional sugar (for example galactose, mannose), sugar chain, or nonpeptide compound. Substances added to the peptide or protein by such modifications can specify or enhance binding to certain receptors or antibodies or otherwise enhance intracellular delivery, activity, half-life, cell- or tissue-specific targeting, or other beneficial properties of the peptide or protein. For example, such modifications can render the peptide or protein more lipophilic, such as may be achieved by addition or conjugation of a phospholipid or fatty acid. Further included within the methods and compositions disclosed herein are peptides and proteins prepared by linkage (for example, chemical bonding) of two or more peptides, protein fragments or functional domains (for example, extracellular, transmembrane and cytoplasmic domains, ligand-binding regions, active site domains, immunogenic epitopes, and the like)—for example fusion peptides and proteins recombinantly produced to incorporate the functional elements of a plurality of different peptides or proteins in a single encoded molecule.

Biologically active peptides and proteins for use within the methods and compositions disclosde herein include native or “wild-type” peptides and proteins and naturally occurring variants of these molecules, such as naturally occurring allelic variants and mutant proteins. Also included are synthetic, such as chemically or recombinantly engineered, peptides and proteins, as well as peptide and protein “analogs” and chemically modified derivatives, fragments, conjugates, and polymers of naturally occurring peptides and proteins. As used herein, the term peptide or protein “analog” is meant to include modified peptides and proteins incorporating one or more amino acid substitutions, insertions, rearrangements or deletions as compared to a native amino acid sequence of a selected peptide or protein, or of a binding domain, fragment, immunogenic epitope, or structural motif, of a selected peptide or protein. Peptide and protein analogs thus modified will be selected for substantially conserved biological activity comparable to that of a corresponding native peptide or protein, or alternatively, reduced or increased biological activity compared to activity exhibited by a corresponding native peptide or protein. For example, analogs, variants, derivatives and mimetics of bacterial products may be selected that exhibit conserved, or substantially increased or decreased activity (compared to the wild-type peptide or protein) for specific binding to one or more nuclear hormone receptor cofactors, proteolytic activity against a nuclear hormone receptor cofactor or other substrate, modulatory activity of a nuclear hormone receptor, immunogenicity, and/or toxicity or activity for induction of inflammatory or autoimmune responses in a mammalian subject. In certain detailed aspects, analogs, variants, derivatives and mimetics of bacterial products are selected that exhibit approximately 20%, 30%, 50%, 85%, 95% or greater activity levels compared to the corresponding native peptide or protein for specific binding to one or more nuclear hormone receptor cofactors, proteolytic activity against a nuclear hormone receptor cofactor or other substrate, modulatory activity of a nuclear hormone receptor, immunogenicity, and/or toxicity or activity for induction of inflammatory or autoimmune responses in a mammalian subject.

As disclosed herein, the term “biologically active peptide or protein analog” further includes derivatives or synthetic variants of a native peptide or protein, such as amino and/or carboxyl terminal deletions and fusions, as well as intrasequence insertions, substitutions or deletions of single or multiple amino acids. Insertional amino acid sequence variants are those in which one or more amino acid residues are introduced into a predetermined site in the protein. Random insertion is also possible with suitable screening of the resulting product. Deletional variants are characterized by removal of one or more amino acids from the sequence. Substitutional amino acid variants are those in which at least one residue in the sequence has been removed and a different residue inserted in its place.

Where a native peptide or protein is modified by amino acid substitution, amino acids are generally replaced by other amino acids having similar, conservatively related chemical properties such as hydrophobicity, hydrophilicity, electronegativity, small or bulky side chains, and the like. Residue positions which are not identical to the native peptide or protein sequence are thus replaced by amino acids having similar chemical properties, such as charge or polarity, where such changes are not likely to substantially effect the properties of the peptide or protein analog. These and other minor alterations will typically substantially maintain biological properties of the modified peptide or protein, including biological activity (such as binding to an adhesion molecule, or other ligand or receptor), immunoidentity (such as recognition by one or more monoclonal antibodies that recognize a native peptide or protein), and other biological properties of the corresponding native peptide or protein.

As used herein, the term “conservative amino acid substitution” refers to the general interchangeability of amino acid residues having similar side chains. For example, a commonly interchangeable group of amino acids having aliphatic side chains is alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Examples of conservative substitutions include the substitution of a non-polar (hydrophobic) residue such as isoleucine, valine, leucine or methionine for another. Likewise, the present disclosure contemplates the substitution of a polar (hydrophilic) residue such as between arginine and lysine, between glutamine and asparagine, and between threonine and serine. Additionally, the substitution of a basic residue such as lysine, arginine or histidine for another or the substitution of an acidic residue such as aspartic acid or glutamic acid for another is also contemplated. Exemplary conservative amino acids substitution groups are: valine-leucine-isoleucine, phenylalanine-tyrosine, lysine-arginine, alanine-valine, and asparagine-glutamine.

The term “biologically active peptide or protein analog” further includes modified forms of a native peptide or protein incorporating stereoisomers (for example, D-amino acids) of the twenty conventional amino acids, or unnatural amino acids such as α,α-disubstituted amino acids, N-alkyl amino acids, lactic acid. These and other unconventional amino acids may also be substituted or inserted within native peptides and proteins useful within the methods and compositions disclosed herein. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-N-methylarginine, and other similar amino acids and imino acids (for example, 4-hydroxyproline). In addition, biologically active peptide or protein analogs include single or multiple substitutions, deletions and/or additions of carbohydrate, lipid and/or proteinaceous moieties that occur naturally or artificially as structural components of the subject peptide or protein, or are bound to or otherwise associated with the peptide or protein.

To facilitate production and use of peptide and protein analogs, reference can be made to molecular phylogenetic studies that characterize conserved and divergent protein structural and functional elements between different members of a species, genus, family or other taxonomic group (such as between bacterial toxins of different species, or allelic or mutant variants of a toxin within a species). In this regard, available studies will provide detailed assessments of structure-function relationships on a fine molecular level for modifying the majority of peptides and proteins disclosed herein to facilitate production and selection of operable peptide and protein analogs, including for a wide range of bacterial toxins and other bacterial products, as well as for cofactors and other agents involved in bacterial toxin-mediated modulation of nuclear hormone receptor activity. These studies may include, for example, detailed sequence comparisons identifying conserved and divergent structural elements among, for example, multiple isoforms or species or allelic variants of a subject bacterial toxin (for example, LF, diptheria toxin, botulinum toxin, or tetanus toxin) or multiple, related bacterial toxins. Such conserved and divergent structural elements facilitate practice of the methods disclosed herein by pointing to useful targets for modifying native peptides and proteins to confer desired structural and/or functional changes.

In this context, existing sequence alignments may be analyzed and conventional sequence alignment methods may be employed to yield sequence comparisons for analysis, for example to identify corresponding protein regions and amino acid positions between protein family members within a species, and between species variants of a protein of interest. These comparisons are useful to identify conserved and divergent structural elements of interest, the latter of which will often be useful for incorporation in a biologically active peptide or protein to yield a functional analog thereof. Typically, one or more amino acid residues marking a divergent structural element of interest in a different reference peptide sequence is incorporated within the functional peptide or protein analog. For example, a cDNA encoding a native LF peptide or protein may be recombinantly modified at one or more corresponding amino acid position(s) (for example, corresponding positions that match or span a similar aligned sequence element according to accepted alignment methods to residues marking the structural element of interest in a heterologous reference peptide or protein sequence, such as an isoform, species or allelic variant, or synthetic mutant, of the subject LF peptide or protein) to encode an amino acid deletion, substitution, or insertion that alters corresponding residue(s) in the native peptide or protein to generate an operable peptide or protein analog of use—having an analogous structural and/or functional element as the reference peptide or protein.

Within this rational design method for constructing biologically active peptide and protein analogs, the native or wild-type identity of residue(s) at amino acid positions corresponding to a structural element of interest in a heterologous reference peptide or protein may be altered to the same, or a conservatively related, residue identity as the corresponding amino acid residue(s) in the reference peptide or protein. However, it is often possible to alter native amino acid residues non-conservatively with respect to the corresponding reference protein residue(s). In particular, many non-conservative amino acid substitutions, particularly at divergent sites suggested to be more amenable to modification, may yield a moderate impairment or neutral effect, or even enhance a selected biological activity, compared to the function of a native peptide or protein.

Sequence alignment and comparisons to forecast useful peptide and protein analogs and mimetics will be further refined by analysis of crystalline structure (see, for example, Löebermann et al., J. Molec. Biol. 177:531-556, 1984; Huber et al., Biochemistry 28:8951-8966, 1989; Stein et al., Nature 347:99-102, 1990; Wei et al., Structural Biology 1:251-255, 1994, each incorporated herein by reference) of native biologically active proteins and peptides, coupled with computer modeling methods known in the art. These analyses allow detailed structure-function mapping to identify desired structural elements and modifications for incorporation into peptide and protein analogs and mimetics that will exhibit substantial activity comparable to that of the native peptide or protein for use within the methods and compositions disclosed herein.

Biologically active peptide and protein analogs as disclosed herein typically show substantial sequence identity to a corresponding native peptide or protein sequence. The term “substantial sequence identity” means that the two subject amino acid sequences, when optimally aligned, such as by the programs GAP or BESTFIT using default gap penalties, share at least 65 percent sequence identity, commonly 80 percent sequence identity, often at least 90-95 percent or greater sequence identity. “Percentage amino acid identity” refers to a comparison of the amino acid sequences of two peptides or proteins which, when optimally aligned, have approximately die designated percentage of the same amino acids. Sequence comparisons are generally made to a reference sequence over a comparison window of at least 10 residue positions, frequently over a window of at least 15-20 amino acids, wherein the percentage of sequence identity is calculated by comparing a reference sequence to a second sequence, the latter of which may represent, for example, a peptide analog sequence that includes one or more deletions, substitutions or additions which total 20 percent, typically less than 5-10% of the reference sequence over the window of comparison. The reference sequence may be a subset of a larger sequence, for example, as a segment of a LF protein. Optimal alignment of sequences for aligning a comparison window may be conducted according to the local homology algorithm of Smith and Waterman (Adv. Appl. Math. 2:482, 1981), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), or by computerized implementations of these algorithms (GAP, BESTFIT, FASTA, and/or TFASTA, such as provided in the Wisconsin Genetics Software Package Release 7.0, Genetics Computer Group, 575 Science Dr., Madison, Wis.).

By aligning a peptide or protein analog optimally with a corresponding native peptide or protein, and by using appropriate assays, such as adhesion protein or receptor binding assays, to determine a selected biological activity, one can readily identify operable peptide and protein analogs for use within the methods and compositions disclosed herein. Operable peptide and protein analogs are typically specifically immunoreactive with antibodies raised to the corresponding native peptide or protein. Likewise, nucleic acids encoding operable peptide and protein analogs will share substantial sequence identity as described above to a nucleic acid encoding the corresponding native peptide or protein, and will typically selectively hybridize to a partial or complete nucleic acid sequence encoding the corresponding native peptide or protein, or fragment thereof, under accepted, moderate or high stringency hybridization conditions (see, for example, Sambrook et al., Molecular Cloning: A Laboratory Manual. 3^{rd} Edition, Cold Spring Harbor Laboratories, Cold Spring Harbor, N.Y., 2001, incorporated herein by reference). The phrase “selectively hybridizing to” refers to a selective interaction between a nucleic acid probe that hybridizes, duplexes or binds preferentially to a particular target DNA or RNA sequence, for example when the target sequence is present in a heterogenous preparation such as total cellular DNA or RNA. Generally, nucleic acid sequences encoding biologically active peptide and protein analogs, or fragments thereof, will hybridize to nucleic acid sequences encoding the corresponding native peptide or protein under stringent conditions (for example, selected to be about 5° C. lower than the thermal melting point (Tm) for the subject sequence at a defined ionic strength and pH, where the Tm is the temperature under defined ionic strength and pH at which 50% of the complementary or target sequence hybridizes to a perfectly matched probe). For discussions of nucleic acid probe design and annealing conditions, see, for example, Sambrook et al., Molecular Cloning: A Laboratory Manual. 3rd Edition, Vols. 1-3, Cold Spring Harbor Laboratory, 2001 or Current Protocols in Molecular Biology, F. Ausubel et al, ed., Greene Publishing and Wiley-Interscience, New York, 1987, each of which is incorporated herein by reference. Typically, stringent or selective conditions will be those in which the salt concentration is at least about 0.02 molar at pH 7 and the temperature is at least about 60° C. Less stringent selective hybridization conditions may also be chosen. As other factors may significantly affect the stringency of hybridization, including, among others, base composition and size of the complementary strands, the presence of organic solvents and the extent of base mismatching, the combination of parameters is more important than the specific measure of any one.

Within additional embodiments, peptide mimetics are provided which comprise a peptide or non-peptide molecule that mimics the tertiary binding structure and activity of a selected native peptide or protein functional domain (for example, binding motif or active site). These peptide mimetics include recombinantly or chemically modified peptides, as well as non-peptide agents such as small molecule drug mimetics, as further described below.

In one aspect, peptides (including polypeptides) of use are modified to produce peptide mimetics by replacement of one or more naturally occurring side chains of the 20 genetically encoded amino acids (or D amino acids) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclics. For example, proline analogs can be made in which the ring size of the proline residue is changed from 5 members to 4, 6, or 7 members. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include the furazanyl, furyl, imidazolidinyl, imidazolyl, imidazolinyl, isothiazolyl, isoxazolyl, morpholinyl (for example morpholino), oxazolyl, piperazinyl (for example 1-piperazinyl), piperidyl (for example 1-piperidyl, piperidino), pyranyl, pyrazinyl, pyrazolidinyl, pyrazolinyl, pyrazolyl, pyridazinyl, pyridyl, pyrimidinyl, pyrrolidinyl (for example 1-pyrrolidinyl), pyrrolinyl, pyrrolyl, thiadiazolyl, thiazolyl, thienyl, thiomorpholinyl (for example thiomorpholino), and triazolyl. These heterocyclic groups can be substituted or unsubstituted. Where a group is substituted, the substituent can be alkyl, alkoxy, halogen, oxygen, or substituted or unsubstituted phenyl.

Peptides and proteins, as well as peptide and protein analogs and mimetics, can also be covalently bound to one or more of a variety of nonproteinaceous polymers, for example, polyethylene glycol, polypropylene glycol, or polyoxyalkenes, in the manner set forth in U.S. Pat. No. 4,640,835; U.S. Pat. No. 4,496,689; U.S. Pat. No. 4,301,144; U.S. Pat. No. 4,670,417; U.S. Pat. No. 4,791,192; or U.S. Pat. No. 4,179,337, all which are incorporated by reference in their entirety herein.

Other peptide and protein analogs and mimetics include glycosylation variants, and covalent or aggregate conjugates with other chemical moieties. Covalent derivatives can be prepared by linkage of functionalities to groups which are found in amino acid side chains or at the N- or C-termini, by means which are well known in the art. These derivatives can include, without limitation, aliphatic esters or amides of the carboxyl terminus, or of residues containing carboxyl side chains, O-acyl derivatives of hydroxyl group-containing residues, and N-acyl derivatives of the amino terminal amino acid or amino-group containing residues, for example, lysine or arginine. Acyl groups are selected from the group of alkyl-moieties including C3 to C18 normal alkyl, thereby forming alkanoyl aroyl species. Covalent attachment to carrier proteins, for example, immunogenic moieties can also be employed.

In addition to these modifications, glycosylation alterations of biologically active peptides and proteins can be made, for example, by modifying the glycosylation patterns of a peptide during its synthesis and processing, or in further processing steps. One means for accomplishing this are by exposing the peptide to glycosylating enzymes derived from cells that normally provide such processing, for example, mammalian glycosylation enzymes. Deglycosylation enzymes can also be successfully employed to yield useful modified peptides and proteins. Also embraced are versions of a native primary amino acid sequence which have other minor modifications, including phosphorylated amino acid residues, for example, phosphotyrosine, phosphoserine, or phosphothreonine, or other moieties, including ribosyl groups or cross-linking reagents.

Peptidomimetics may also have amino acid residues that have been chemically modified by phosphorylation, sulfonation, biotinylation, or the addition or removal of other moieties, particularly those that have molecular shapes similar to phosphate groups. In some embodiments, the modifications will be useful labeling reagents, or serve as purification targets, for example, affinity ligands.

A major group of peptidomimetics comprises covalent conjugates of native peptides or proteins, or fragments thereof, with other proteins or peptides. These derivatives can be synthesized in recombinant culture such as N- or C-terminal fusions or by the use of agents known in the art for their usefulness in cross-linking proteins through reactive side groups. Preferred peptide and protein derivatization sites for targeting by cross-linking agents are at free amino groups, carbohydrate moieties, and cysteine residues.

Fusion polypeptides between biologically active peptides or proteins and other homologous or heterologous peptides and proteins are also provided. Many growth factors and cytokines are homodimeric entities, and a repeat construct of these molecules or active fragments thereof will yield various advantages, including lessened susceptibility to proteolytic degradation. Repeat and other fusion constructs of bacterial proteins and peptides yield similar advantages within the methods and compositions disclosed herein. Various alternative multimeric constructs comprising peptides and proteins of use are thus provided. In certain embodiments, biologically active polypeptide fusions are provided as described in U.S. Pat. Nos. 6,018,026, 5,843,725, 6,291,646, 6,300,099, and 6,323,323 (each incorporated herein by reference), for example by linking one or more biologically active peptides or proteins disclosed herein with a heterologous, multimerizing polypeptide or protein, for example an immunoglobulin heavy chain constant region, or an immunoglobulin light chain constant region. The biologically active, multimerized polypeptide fusion thus constructed can be a hetero- or homo-multimer, for example, a heterodimer or homodimer comprising one or more bacterial proteins or peptides(s), which can each include one or more distinct biologically active peptides or proteins operable within the methods and compositions disclosed herein. Other heterologous polypeptides can be combined with the active peptide or protein to yield fusions that exhibit a combination of properties or activities of the derivative proteins. Other typical examples are fusions of a reporter polypeptide, for example, CAT or luciferase, with a peptide or protein as described herein, to facilitate localization of the fused peptide or protein (see, for example, Dull et al., U.S. Pat. No. 4,859,609, incorporated herein by reference). Other fusion partners useful in this context include bacterial beta-galactosidase, trpE, Protein A, beta-lactamase, alpha amylase, alcohol dehydrogenase, and yeast alpha mating factor (see, for example, Godowski et al., Science 241:812-816, 1988, incorporated herein by reference).

The use of biologically active peptides and proteins modified by covalent or aggregative association with chemical moieties van also be used in the methods disclosed herein. These derivatives generally fall into the three classes: (1) salts, (2) side chain and terminal residue covalent modifications, and (3) adsorption complexes, for example with cell membranes. Such covalent or aggregative derivatives are useful for various purposes, for example as agonists or antagonists to native bacterial products, as immunogens, as reagents in immunoassays, or in purification methods such as for affinity purification of ligands or other binding ligands. For example, an active peptide or protein can be immobilized by covalent bonding to a solid support such as cyanogen bromide-activated Sepharose, by methods which are well known in the art, or adsorbed onto polyolefin surfaces, with or without glutaraldehyde cross-linking, for use in the assay or purification of antibodies that specifically bind the active peptide or protein. The active peptide or protein can also be labeled with a detectable group, for example radioiodinated by the chloramine T procedure, covalently bound to rare earth chelates, or conjugated to another fluorescent moiety for use in diagnostic assays, including assays involving in vivo administration of the labeled peptide or protein to determine, such as nuclear hormone receptor activity, succeptibility to a disease or condition associated with bacterial infection, or other related indicia.

Those of skill in the art recognize that a variety of techniques are available for constructing peptide and protein mimetics with the same, similar, increased, or reduced biological activity as the corresponding native peptide or protein. Often these analogs, variants, derivatives and mimetics will exhibit one or more desired activities that are distinct or improved from the corresponding native peptide or protein, for example improved characteristics of solubility, stability, and/or susceptibility to hydrolysis or proteolysis (see, for example, Morgan and Gainor, Ann. Rep. Med. Chem. 24:243-252, 1989, incorporated herein by reference). Certain peptidomimetic compounds are based upon the amino acid sequence of the proteins and peptides described herein, including sequences of bacterial toxins such as LF. Typically, peptidomimetic compounds are synthetic compounds having a three-dimensional structure (of at least part of the mimetic compound) that mimics, for example, the primary, secondary, and/or tertiary structural, and/or electrochemical characteristics of a selected peptide or protein, or a structural domain, active site, or binding region (for example, a homotypic or heterotypic binding site, catalytic active site or domain, receptor or ligand binding interface or domain, etc.) thereof. The peptide-mimetic structure or partial structure (also referred to as a peptidomimetic “motif” of a peptidomimetic compound) will often share a desired biological activity with a native peptide or protein, as discussed above (for example, receptor or cofactor binding and/or activation or repression activities, immunogenic activity (such as binding to MHC molecules of one or multiple haplotypes and activating CD8⁺ and/or CD4⁺ T), etc. Typically, at least one subject biological activity of the mimetic compound is not substantially reduced in comparison to, and is often the same as or greater than, the activity of the native peptide on which the mimetic was modeled. In addition, peptidomimetic compounds can have other desired characteristics that enhance their therapeutic application, such as increased cell permeability, greater affinity and/or avidity, and prolonged biological half-life. The peptidomimetics will sometimes have a “backbone” that is partially or completely non-peptide, but with side groups identical to the side groups of the amino acid residues that occur in the peptide or protein on which the peptidomimetic is modeled. Several types of chemical bonds, for example ester, thioester, thioamide, retroamide, reduced carbonyl, dimethylene and ketomethylene bonds, are known in the art to be generally useful substitutes for peptide bonds in the construction of protease-resistant peptidomimetics.

The following describes methods for preparing peptide and protein mimetics modified at the N-terminal amino group, the C-terminal carboxyl group, and/or changing one or more of the amido linkages in the peptide to a non-amido linkage. It being understood that two or more such modifications can be coupled in one peptide or protein mimetic structure (for example, modification at the C-terminal carboxyl group and inclusion of a —CH₂-carbamate linkage between two amino acids in the peptide. For N-terminal modifications, peptides typically are synthesized as the free acid but, as noted above, can be readily prepared as the amide or ester. One can also modify the amino and/or carboxy terminus of peptide compounds to produce other compounds of use. Amino terminus modifications include methylating (for example, —NHCH₃ or —NH(CH₃)₂), acetylating, adding a carbobenzoyl group, or blocking the amino terminus with any blocking group containing a carboxylate functionality defined by RCOO—, where R is selected from the group consisting of naphthyl, acridinyl, steroidyl, and similar groups. Carboxy terminus modifications include replacing the free acid with a carboxamide group or forming a cyclic lactam at the carboxy terminus to introduce structural constraints. Amino terminus modifications are as recited above and include alkylating, acetylating, adding a carbobenzoyl group, forming a succinimide group, etc. The N-terminal amino group can then be reacted as follows:
(a) to form an amide group of the formula RC(O)NH— where R is as defined above by reaction with an acid halide [for example, RC(O)Cl] or acid anhydride. Typically, the reaction can be conducted by contacting about equimolar or excess amounts (for example, about 5 equivalents) of an acid halide to the peptide in an inert diluent (for example, dichloromethane) preferably containing an excess (for example, about 10 equivalents) of a tertiary amine, such as diisopropylethylamine, to scavenge the acid generated during reaction. Reaction conditions are otherwise conventional (for example, room temperature for 30 minutes). Alkylation of the terminal amino to provide for a lower alkyl N-substitution followed by reaction with an acid halide as described above will provide for N-alkyl amide group of the formula RC(O)NR—;
(b) to form a succinimide group by reaction with succinic anhydride. As before, an approximately equimolar amount or an excess of succinic anhydride (for example, about 5 equivalents) can be employed and the amino group is converted to the succinimide by methods well known in the art including the use of an excess (for example, ten equivalents) of a tertiary amine such as diisopropylethylamine in a suitable inert solvent (for example, dichloromethane) (see, for example, Wollenberg, et al., U.S. Pat. No. 4,612,132, incorporated herein by reference). It is understood that the succinic group can be substituted with, for example, C₂-C₆ alky or —SR substituents that are prepared in a conventional manner to provide for substituted succinimide at the N-terminus of the peptide. Such alkyl substituents are prepared by reaction of a lower olefin (C₂-C₆) with maleic anhydride in the manner described by Wollenberg, et al. (U.S. Pat. No. 4,612,132) and —SR substituents are prepared by reaction of RSH with maleic anhydride where R is as defined above;
(c) to form a benzyloxycarbonyl-NH— or a substituted benzyloxycarbonyl-NH— group by reaction with approximately an equivalent amount or an excess of CBZ-CL (for example, benzyloxycarbonyl chloride) or a substituted CBZ-Cl in a suitable inert diluent (for example, dichloromethane) preferably containing a tertiary amine to scavenge the acid generated during the reaction;
(d) to form a sulfonamide group by reaction with an equivalent amount or an excess (for example, 5 equivalents) of R—S(O)₂Cl in a suitable inert diluent (dichloromethane) to convert the terminal amine into a sulfonamide where R is as defined above. Preferably, the inert diluent contains excess tertiary amine (for example, ten equivalents) such as diisopropylethylamine, to scavenge the acid generated during reaction. Reaction conditions are otherwise conventional (for example, room temperature for 30 minutes);
(e) to form a carbamate group by reaction with an equivalent amount or an excess (for example, 5 equivalents) of R—OC(O)Cl or R—OC(O)OC₆H₄-p-NO₂ in a suitable inert diluent (for example, dichloromethane) to convert the terminal amine into a carbamate where R is as defined above. Preferably, the inert diluent contains an excess (for example, about 10 equivalents) of a tertiary amine, such as diisopropylethylamine, to scavenge any acid generated during reaction. Reaction conditions are otherwise conventional (for example, room temperature for 30 minutes);
(f) to form a urea group by reaction with an equivalent amount or an excess (for example, 5 equivalents) of R—N═C═O in a suitable inert diluent (for example, dichloromethane) to convert the terminal amine into a urea (for example, RNHC(O)NH—) group where R is as defined above. Preferably, the inert diluent contains an excess (for example, about 10 equivalents) of a tertiary amine, such as diisopropylethylamine. Reaction conditions are otherwise conventional (for example, room temperature for about 30 minutes).

In preparing peptide mimetics wherein the C-terminal carboxyl group is replaced by an ester (for example, —C(O)OR where R is as defined above), resins as used to prepare peptide acids are typically employed, and the side chain protected peptide is cleaved with base and the appropriate alcohol, for example, methanol. Side chain protecting groups are then removed in the usual fashion by treatment with hydrogen fluoride to obtain the desired ester.

In preparing peptide mimetics wherein the C-terminal carboxyl group is replaced by the amide —C(O)NR₃R₄, a benzhydrylamine resin is used as the solid support for peptide synthesis. Upon completion of the synthesis, hydrogen fluoride treatment to release the peptide from the support results directly in the free peptide amide (for example, the C-terminus is —C(O)NH₂). Alternatively, use of the chloromethylated resin during peptide synthesis coupled with reaction with ammonia to cleave the side chain protected peptide from the support yields the free peptide amide and reaction with an alkylamine or a dialkylamine yields a side chain protected alkylamide or dialkylamide (for example, the C-terminus is —C(O)NRR₁ where R and R₁ are as defined above). Side chain protection is then removed in the usual fashion by treatment with hydrogen fluoride to give the free amides, alkylamides, or dialkylamides.

In another alternative embodiments, the C-terminal carboxyl group or a C-terminal ester of a biologically active peptide can be induced to cyclize by internal displacement of the —OH or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine. Internal cyclization as opposed to polymerization can be enhanced by use of very dilute solutions. Such methods are well known in the art.

One can cyclize active peptides for use, or incorporate a desamino or descarboxy residue at the termini of the peptide, so that there is no terminal amino or carboxyl group, to decrease susceptibility to proteases, or to restrict the conformation of the peptide. C-terminal functional groups among peptide analogs and mimetics include amide, amide lower alkyl, amide di(lower alkyl), lower alkoxy, hydroxy, and carboxy, and the lower ester derivatives thereof, and the pharmaceutically acceptable salts thereof.

Other methods for making peptide and protein derivatives and mimetics for use within the methods and compositions disclosed herein are described in Hruby et al. (Biochem J. 268(2:249-262, 1990, incorporated herein by reference). According to these methods, biologically active peptides and proteins serve as structural models for non-peptide mimetic compounds having similar biological activity as the native peptide or protein. Those of skill in the art recognize that a variety of techniques are available for constructing compounds with the same or similar desired biological activity as the lead peptide or protein compound, or that have more favorable activity than the lead with respect a desired property such as solubility, stability, and susceptibility to hydrolysis and proteolysis (see, for example, Morgan and Gainor, Ann. Rep. Med. Chem. 24:243-252, 1989, incorporated herein by reference). These techniques include, for example, replacing a peptide backbone with a backbone composed of phosphonates, amidates, carbamates, sulfonamides, secondary amines, and/or N-methylamino acids.

Peptide and protein mimetics wherein one or more of the peptidyl linkages [—C(O)NH—] have been replaced by such linkages as a —CH₂-carbamate linkage, a phosphonate linkage, a —CH₂-sulfonamide linkage, a urea linkage, a secondary amine (—CH₂NH—) linkage, and an alkylated peptidyl linkage [—C(O)NR₆— where R₆ is lower alkyl] are prepared, for example, during conventional peptide synthesis by merely substituting a suitably protected amino acid analogue for the amino acid reagent at the appropriate point during synthesis. Suitable reagents include, for example, amino acid analogues wherein the carboxyl group of the amino acid has been replaced with a moiety suitable for forming one of the above linkages. For example, if one desires to replace a —C(O)NR— linkage in the peptide with a —CH₂-carbamate linkage (—CH₂OC(O)NR—), then the carboxyl (—COOH) group of a suitably protected amino acid is first reduced to the —CH₂OH group which is then converted by conventional methods to a —OC(O)Cl functionality or a para-nitrocarbonate —OC(O)O—C₆H₄-p-NO₂ functionality. Reaction of either of such functional groups with the free amine or an alkylated amine on the N-terminus of the partially fabricated peptide found on the solid support leads to the formation of a —CH₂OC(O)NR— linkage. For a more detailed description of the formation of such —CH₂-carbamate linkages, see, for example, Cho et al. (Science 261:1303-1305, 1993, incorporated herein by reference).

Replacement of an amido linkage in an active peptide with a —CH₂-sulfonamide linkage can be achieved by reducing the carboxyl (—COOH) group of a suitably protected amino acid to the —CH₂OH group, and the hydroxyl group is then converted to a suitable leaving group such as a tosyl group by conventional methods. Reaction of the derivative with, for example, thioacetic acid followed by hydrolysis and oxidative chlorination will provide for the —CH₂—S(O)₂Cl functional group which replaces the carboxyl group of the otherwise suitably protected amino acid. Use of this suitably protected amino acid analogue in peptide synthesis provides for inclusion of an —CH₂S(O)₂NR— linkage that replaces the amido linkage in the peptide thereby providing a peptide mimetic. For a more complete description on the conversion of the carboxyl group of the amino acid to a —CH₂S(O)₂Cl group, see, for example, Weinstein and Boris (Chemistry & Biochemistry of Amino Acids. Peptides and Proteins, Vol. 7, pp. 267-357, Marcel Dekker, Inc., New York, 1983, incorporated herein by reference). Replacement of an amido linkage in an active peptide with a urea linkage can be achieved, for example, in the manner set forth in U.S. patent application Ser. No. 08/147,805 (incorporated herein by reference).

Secondary amine linkages wherein a—CH₂NH— linkage replaces the amido linkage in the peptide can be prepared by employing, for example, a suitably protected dipeptide analogue wherein the carbonyl bond of the amido linkage has been reduced to a CH₂ group by conventional methods. For example, in the case of diglycine, reduction of the amide to the amine will yield after deprotection H₂NCH₂CH₂NHCH₂ COOH that is then used in N-protected form in the next coupling reaction. The preparation of such analogues by reduction of the carbonyl group of the amido linkage in the dipeptide is well known in the art.

The biologically active peptide and protein agents of the present disclosure can exist in a monomeric form with no disulfide bond formed with the thiol groups of cysteine residue(s) that may be present in the subject peptide or protein. Alternatively, an intermolecular disulfide bond between thiol groups of cysteines on two or more peptides or proteins can be produced to yield a multimeric (for example, dimeric, tetrameric or higher oligomeric) compound. Certain of such peptides and proteins can be cyclized or dimerized via displacement of the leaving group by the sulfur of a cysteine or homocysteine residue (see, for example, Barker et al., J. Med. Chem. 35:2040-2048, 1992; and Or et al., J. Org. Chem. 56:3146-3149, 1991, each incorporated herein by reference). Thus, one or more native cysteine residues may be substituted with a homocysteine. Intramolecular or intermolecular disulfide derivatives of active peptides and proteins provide analogs in which one of the sulfurs has been replaced by a CH₂ group or other isostere for sulfur. These analogs can be made via an intramolecular or intermolecular displacement, using methods known in the art.

Within certain embodiments, delivery of biologically active agents, including native bacterial products and analogs, variants, derivatives and mimetics thereof, is enhanced by methods and agents that target selective transport mechanisms and promote endo- or transcytocis of macromoloecular drugs. In this regard, the compositions and delivery methods optionally incorporate a selective transport-enhancing agent that facilitates transport of one or more biologically active agents. These transport-enhancing agents can be employed in a combinatorial formulation or coordinate administration protocol with one or more of the peptides, proteins, analogs and mimetics disclosed herein, to coordinately enhance delivery of the biologically active agent(s) into target cells. Exemplary selective transport-enhancing agents for use within this aspect include, but are not limited to, glycosides, sugar-containing molecules, and binding agents such as lectin binding agents, which are known to interact specifically with epithelial transport barrier components (see, for example, Goldstein et al., Annu. Rev. Cell. Biol. 1:1-39, 1985, incorporated herein by reference). For example, specific “bioadhesive” ligands, including various plant and bacterial lectins, which bind to cell surface sugar moieties by receptor-mediated interactions can be employed as carriers or conjugated transport mediators for enhancing delivery of biologically active agents. Certain bioadhesive ligands of use will mediate transmission of biological signals to epithelial target cells that trigger selective uptake of the adhesive ligand by specialized cellular transport processes (endocytosis or transcytosis). These transport mediators can therefore be employed as a “carrier system” to stimulate or direct selective uptake of one or more biologically active agent(s) within the methods disclosed herein. To utilize these transport-enhancing agents, general carrier formulation and/or conjugation methods known in the art are used to coordinately administer a selective transport enhancer (for example, a receptor-specific ligand) and a biologically active agent to a subject to trigger or mediate enhanced endo- or transcytosis of the active agent into specific target cell(s), tissue(s) or compartment(s).

“Lectins” are plant proteins that bind to specific sugars found on the surface of glycoproteins and glycolipids of eukaryotic cells. Concentrated solutions of lectins have a ‘mucotractive’ effect, and various studies have demonstrated rapid receptor mediated endocytocis (RME) of lectins and lectin conjugates (for example, concanavalin A conjugated with colloidal gold particles) across mucosal surfaces. Additional studies have reported that the uptake mechanisms for lectins can be utilized for intestinal drug targeting in vivo. In certain of these studies, polystyrene nanoparticles (500 nm) were covalently coupled to tomato lectin and reported yielded improved systemic uptake after oral administration to rats.

In addition to plant lectins, microbial adhesion and invasion factors provide a rich source of candidates for use as adhesive/selective transport carriers within the compositions and methods disclosed herein (see, for example, Lehr, Crit. Rev. Therap. Drug Carrier Syst. 11:177-218, 1995; Swann, Pa., Pharmaceutical Research 15:826-832, 1998, each incorporated herein by reference). Two components are necessary for bacterial adherence processes, a bacterial ‘adhesin’ (adherence or colonization factor) and a receptor on the host cell surface. Bacteria causing mucosal infections need to penetrate the mucus layer before attaching themselves to the epithelial surface. This attachment is usually mediated by bacterial fimbriae or pilus structures, although other cell surface components may also take part in the process. Adherent bacteria colonize mucosal epithelia by multiplication and initiation of a series of biochemical reactions inside the target cell through signal transduction mechanisms (with or without the help of toxins). Associated with these invasive mechanisms, a wide diversity of bioadhesive proteins (for example, invasin, internalin) originally produced by various bacteria and viruses are known. These allow for extracellular attachment of such microorganisms with an impressive selectivity for host species and even particular target tissues. Signals transmitted by such receptor-ligand interactions trigger the transport of intact, living microorganisms into, and eventually through, epithelial cells by endo- and transcytotic processes. Such naturally occurring phenomena may be harnessed (for example, by complexing biologically active agents such as bacterial toxin with an adhesin) according to the teachings herein for enhanced delivery of biologically active compounds to target sites of drug action. One advantage of this strategy is that the selective carrier partners thus employed are substrate-specific, leaving the natural barrier function of epithelial tissues intact against other solutes (see, for example, Lehr, Drug Absorption Enhancement, pp. 325-362, de Boer, Ed., Harwood Academic Publishers, 1994, incorporated herein by reference).

Various bacterial and plant toxins that bind epithelial surfaces in a specific, lectin-like manner are also useful within the methods and compositions disclosed herein. For example, diptheria toxin (DT) enters host cells rapidly by RME. Likewise, the B subunit of the E. coli heat labile toxin binds to the brush border of intestinal epithelial cells in a highly specific, lectin-like manner. Uptake of this toxin and transcytosis to the basolateral side of the enterocytes has been reported in vivo and in vitro. Other researches have expressed the transmembrane domain of diphtheria toxin in E. coli as a maltose-binding fusion protein and coupled it chemically to high-Mw poly-L-lysine. The resulting complex was successfully used to mediate internalization of a reporter gene in vitro. In addition to these examples, Staphylococcus aureus produces a set of proteins (for example, staphylococcal enterotoxin A (SEA), SEB, toxic shock syndrome toxin 1 (TSST-1) which act both as superantigens and toxins. Studies relating to these proteins have reported dose-dependent, facilitated transcytosis of SEB and TSST-I in Caco-2 cells.

Various plant toxins, mostly ribosome-inactivating proteins (RIPs), have been identified that bind to any mammalian cell surface expressing galactose units and are subsequently internalized by RME. Toxins such as nigrin b, α-sarcin, ricin and saporin, viscumin, and modeccin are highly toxic upon oral administration (for example, are rapidly internalized). Therefore, modified, less toxic subunits of these compound will be useful to facilitate the uptake of biologically active agents, including bacterial products and analogs, variants, derivatives and mimetics thereof.

Viral haemagglutinins comprise another type of transport agent to facilitate delivery of biologically active agents within the methods and compositions disclosed herein. The initial step in many viral infections is the binding of surface proteins (haemagglutinins) to mucosal cells. These binding proteins have been identified for most viruses, including rotaviruses, varicella zoster virus, semliki forest virus, adenoviruses, potato leafroll virus, and reovirus. These and other exemplary viral hemagglutinins can be employed in a combinatorial formulation (for example, a mixture or conjugate formulation) or coordinate administration protocol with, for example, one or more bacterial products or analogs, variants, derivatives and mimetics thereof. Alternatively, viral hemagglutinins can be employed in a combinatorial formulation or coordinate administration protocol to directly enhance delivery of a biologically active agent.

A variety of endogenous, selective transport-mediating factors are also available for use within the methods and compositions disclosed herein. Mammalian cells have developed an assortment of mechanisms to facilitate the internalization of specific substrates and target these to defined compartments. Collectively, these processes of membrane deformations are termed ‘endocytosis’ and comprise phagocytosis, pinocytosis, receptor-mediated endocytosis (clathrin-mediated RME), and potocytosis (non-clathrin-mediated RME). RME is a highly specific cellular biologic process by which, as its name implies, various ligands bind to cell surface receptors and are subsequently internalized and trafficked within the cell. In many cells the process of endocytosis is so active that the entire membrane surface is internalized and replaced in less than a half hour.

RME is initiated when specific ligands bind externally oriented membrane receptors. Binding occurs quickly and is followed by membrane invagination until an internal vesicle forms within the cell (the early endosome, “receptosome”, or CURL (compartment of uncoupling receptor and ligand). Localized membrane proteins, lipids and extracellular solutes are also internalized during this process. When the ligand binds to its specific receptor, the ligand-receptor complex accumulates in coated pits. Coated pits are areas of the membrane with high concentration of endocellular clathrin subunits. The assembly of clathrin molecules on the coated pit is believed to aid the invagination process. Specialized coat proteins called adaptins, trap specific membrane receptors that move laterally through the membrane in the coated pit area by binding to a signal sequence (Tyr-X-Arg-Phe, where X=any amino acid) at the endocellular carboxy terminus of the receptor. This process ensures that the correct receptors are concentrated in the coated pit areas and minimizes the amount of extracellular fluid that is taken up in the cell.

Following the internalization process, the clathrin coat is lost through the help of chaperone proteins, and proton pumps lower the endosomal pH to approximately 5.5, which causes dissociation of the receptor-ligand complex. CURL serves as a compartment to segregate the recycling receptor (for example transferrin) from receptor involved in transcytosis (for example transcoba-lamin). Endosomes may then move randomly or by saltatory motion along the microtubules until they reach the trans-Golgi reticulum where they are believed to fuse with Golgi components or other membranous compartments and convert into tubulovesicular complexes and late endosomes or multivesicular bodies. The fate of the receptor and ligand are determined in these sorting vesicles. Some ligands and receptors are returned to the cell surface where the ligand is released into the extracellular milieu and the receptor is recycled. Alternatively, the ligand is directed to lysosomes for destruction while the receptor is recycled to the cell membrane. The endocytotic recycling pathways of polarized epithelial cells are generally more complex than in non-polarized cells. In these enterocytes a common recycling compartment exists that receives molecules from both apical and basolateral membranes and is able to correctly return them to the appropriate membrane or membrane recycling compartment

Current understanding of RME receptor structure and related structure-function relationships has been significantly enhanced by the cloning of mRNA sequences coding for endocytotic receptors. Most RME receptors share principal structural features, such as an extracellular ligand binding site, a single hydrophobic transmembrane domain (unless the receptor is expressed as a dimer), and a cytoplasmic tail encoding endocytosis and other functional signals. Two classes of receptors are proposed based on their orientation in the cell membrane; the amino terminus of Type I receptors is located on the extracellular side of the membrane, whereas Type II receptors have this same protein tail in the intracellular milieu.

As noted above, potocytosis, or non-clathrin coated endocytosis, takes place through caveolae, which are uniform omega- or flask-shaped membrane invaginations 50-80 nm in diameter. This process was first described as the internalization mechanism of the vitamin folic acid. Morphological studies have implicated caveolae in i) the transcytosis of macromolecules across endothelial cells; (ii) the uptake of small molecules via potocytosis involving GPI-linked receptor molecules and an unknown anion transport protein; iii) interactions with the actin-based cytoskeleton; and (iv) the compartmentalization of certain signaling molecules involved in signal transduction, including G-protein coupled receptors. Caveolae are characterized by the presence of an integral 22-kDa membrane protein termed VIP21-caveolin, which coats the cytoplasmic surface of the membrane. From a drug delivery standpoint, the advantage of potocytosis pathways over clathrin-coated RME pathways lies in the absence of the pH lowering step, which circumvents the endosomal/lysosomal pathway. This pathway for selective transporter-mediated delivery of biologically active agents is therefore particularly effective for enhanced delivery of pH-sensitive macromolecules.

Exemplary among potocytotic transport carriers mechanisms for use is the folate carrier system, which mediates transport of the vitamin folic acid (FA) into target cells via specific binding to the folate receptor (FR) (see, for example, Reddy et al., Crit. Rev. Ther. Drug Car. Syst. 15:587-627, 1998, incorporated herein by reference). The cellular uptake of free folic acid is mediated by the folate receptor and/or the reduced folate carrier. The folate receptor is a glycosylphosphatidylinositol (GPI)-anchored 38 kDa glycoprotein clustered in caveolae mediating cell transport by potocytosis. While the expression of the reduced folate carrier is ubiquitously distributed in eukaryotic cells, the folate receptor is principally overexpressed in human tumors. Two homologous isoforms (α and β) of the receptor have been identified in humans. The α-isoform is found to be frequently overexprssed in epithelial tumors, whereas the β-form is often found in non-epithelial lineage tumors. Consequently, this receptor system has been used in drug-targeting approaches to cancer cells, but also in protein delivery, gene delivery, and targeting of antisense oligonucleotides to a variety of cell types.

Folate-drug conjugates are well suited for use within the methods and compositions disclosed herein, because they allow penetration of target cells exclusively via FR-mediated endocytosis. When FA is covalently linked, for example, via its γ-carboxyl to a biologically active agent, FR binding affinity (KD˜10⁻¹⁰M) is not significantly compromised, and endocytosis proceeds relatively unhindered, promoting uptake of the attached active agent by the FR-expressing cell. Because FRs are significantly overexpressed on a large fraction of human cancer cells (for example, ovarian, lung, breast, endometrial, renal, colon, and cancers of myeloid hematopoietic cells), this methodology allows for selective delivery of a wide range of therapeutic as well as diagnostic agents to tumors. Folate-mediated tumor targeting has been exploited to date for delivery of the following classes of molecules and molecular complexes: (i) protein toxins, (ii) low-molecular-weight chemotherapeutic agents, (iii) radioimaging agents, (iv) MRI contrast agents, (v) radio-therapeutic agents, (vi) liposomes with entrapped drugs, (vii) genes, (viii) antisense oligonucleotides, (ix) ribozymes, and (x) immunotherapeutic agents (see, for example, Swann, Pa., Pharmaceutical Research 15:826-832, 1998, incorporated herein by reference). In virtually all cases, in vitro studies demonstrate a significant improvement in potency and/or cancer-cell specificity over the nontargeted form of the same pharmaceutical agent.

In addition to the folate receptor pathway, a variety of additional methods to stimulate transcytosis within the disclosed methods are directed to the transferrin receptor pathway, and the riboflavin receptor pathway. In one aspect, conjugation of a biologically active agent to riboflavin can effectuate RME-mediated uptake. Yet additional embodiments utilize vitamin B12 (cobalamin) as a specialized transport protein (for example, conjugation partner) to facilitate entry of biologically active agents into target cells. Certain studies suggest that this particular system can be employed for the intestinal uptake of luteinizing hormone releasing factor (LHRH)-analogs, granulocyte colony stimulating factor (G-CSF, 18.8 kDa), erythropoietin (29.5 kDa), α-interferon, and the LHRH-antagonist ANTIDE.

Still other embodiments utilize transferrin as a carrier or stimulant of RME of mucosally delivered biologically active agents. Transferrin, an 80 kDa iron-transporting glycoprotein, is efficiently taken up into cells by RME. Transferrin receptors are found on the surface of most proliferating cells, in elevated numbers on erythroblasts and on many kinds of tumors. According to current knowledge of intestinal iron absorption, transferrin is excreted into the intestinal lumen in the form of apotransferrin and is highly stable to attacks from intestinal peptidases. In most cells, diferric transferrin binds to transferrin receptor (TfR), a dimeric transmembrane glycoprotein of 180 kDa, and the ligand-receptor complex is endocytosed within clathrin-coated vesicles. After acidification of these vesicles, iron dissociates from the transferrin/TfR complex and enters the cytoplasm, where it is bound by ferritin (Fn). Recent reports suggest that insulin covalently coupled to transferrin, is transported across Cac6-2 cell monolayers by RME. Other studies suggest that oral administration of this complex to streptozotocin-induced diabetic mice significantly reduces plasma glucose levels (28%), which is further potentiated by BFA pretreatment The transcytosis of transferrin (Tf) and transferrin conjugates is reportedly enhanced in the presence of Brefeldin A (BFA), a fungal metabolite. In other studies, BFA treatment has been reported to rapidly increase apical endocytosis of both ricin and HRP in MDCK cells. Thus, BFA and other agents that stimulate receptor-mediated transport can be employed within the methods disclosed herein as combinatorially formulated (for example, conjugated) and/or coordinately administered agents to enhance receptor-mediated transport of biologically active agents, including, for example, bacterial toxins and analogs, variants, derivatives and mimetics thereof.

Immunoglobulin transport mechanisms provide yet additional endogenous pathways and reagents for incorporation within the methods and compositions disclosed herein. Receptor-mediated transcytosis of immunoglobulin G (IgG) across the neonatal small intestine serves to convey passive immunity to many newborn mammals. In rats, IgG in milk selectively binds to neonatal Fc receptors (FcRn) expressed on the surface of the proximal small intestinal enterocytes during the first three weeks after birth. FcRn binds IgG in a pH-dependent manner, with binding occurring at the luminal pH (approx. 6-6.5) of the jejunum and release at the pH of plasma (approx. 7.4). The Fc receptor resembles the major histocompatibility complex (MHC) class I antigens in that it consists of two subunits, a transmembrane glycoprotein (gp50) in association with β2-microglobulin. In mature absorptive cells both subunits are colocalized in each of the membrane compartments that mediate transcytosis of IgG. IgG administered in situ apparently causes both subunits to concentrate within endocytic pits of the apical plasma membrane, suggesting that ligand causes redistribution of receptors at this site. These results support a model for transport in which IgG is transferred across the cell as a complex with both subunits.

Within the methods and compositions disclosed herein, IgG and other immune system-related carriers (including polyclonal and monoclonal antibodies and various fragments thereof) can be coordinately administered with biologically active agents to provide for targeted delivery, typically by receptor-mediated transport, of the biologically active agent. For example, the biologically active agent may be covalently linked to the IgG or other immunological active agent or, alternatively, formulated in liposomes or other carrier vehicle which is in turn modified (such as coated or covalently linked) to incorporate IgG or other immunological transport enhancer. In certain embodiments, polymeric IgA and/or IgM transport agents are employed, which bind to the polymeric immunoglobulin receptors (pIgRs) of target epithelial cells. Within these methods, expression of pIgR can be enhanced by cytokines.

Within other embodiments, antibodies and other immunological transport agents can themselves be modified for enhanced delivery of biologically active agents. For example, antibodies can be more effectively administered by charge modifying techniques. In one such aspect, an antibody drug delivery strategy involving antibody cationization is utilized that facilitates both trans-endothelial migration and target cell endocytosis (see, for example, Pardridge, et al., JPET 286:548-544, 1998, incorporated herein by reference). In one such strategy, the pI of the antibody is increased by converting surface carboxyl groups of the protein to extended primary amino groups. These canonized homologous proteins have no measurable tissue toxicity and have minimal immunogenicity. In addition, monoclonal antibodies may be cationized with retention of affinity for the target protein.

Additional selective transport-enhancing agents for use within the methods disclosed herein comprise whole bacteria and viruses, including genetically engineered bacteria and viruses, as well as components of such bacteria and viruses. In addition to conventional gene delivery vectors (for example, adenovirus) and related methods, this aspect includes the use of bacterial ghosts and subunit constructs, for example, as described by Huter et al., Journal of Controlled Release 61:51-63, 1999 (incorporated herein by reference). Bacterial ghosts are non-denatured bacterial cell envelopes, for example as produced by the controlled expression of the plasmid-encoded lysis gene E of bacteriophage PhiXI74 in gram-negative bacteria. Protein E-specific lysis does not cause any physical or chemical denaturation to bacterial surface structures, and bacterial ghosts are therefore useful in development of inactivated whole-cell vaccines. Ghosts produced from Actinobacillus pleuropneumoniae, Pasteurella haemolytica and Salmonella sp. have proved successful in vaccination experiments. Recombinant bacterial ghosts can be created by the expression of foreign genes fused to a membrane-targeting sequence, and thus can carry foreign therapeutic peptides and proteins anchored in their envelope. The fact that bacterial ghosts preserve a native cell wall, including bioadhesive structures like fimbriae of their living counterparts, makes them suitable for the attachment to specific target tissues such as mucosal surfaces. Bacterial ghosts have been shown to be readily taken up by macrophages, thus adhesion of ghosts to specific tissues can be followed by uptake through phagocytes.

In view of the foregoing, a wide variety of ligands involved in receptor-mediated transport mechanisms are known in the art and can be variously employed within the methods and compositions disclosed herein (for example, as conjugate partners or coordinately administered mediators) to enhance receptor-mediated transport of biologically active agents, including various bacterial products, cofactors and other active agents disclosed herein, and analogs, variants, derivatives and mimetics thereof. Generally, these ligands include hormones and growth factors, bacterial adhesins and toxins, lectins, metal ions and their carriers, vitamins, immunoglobulins, whole viruses and bacteria or selected components thereof. Exemplary ligands among these classes include, for example, calcitonin, prolactin, epidermal growth factor, glucagon, growth hormone, estrogen, lutenizing hormone, platelet derived growth factor, thyroid stimulating hormone, thyroid hormone, cholera toxin, diptheria toxin, E. coli heat labile toxin, Staphylococcal enterotoxins A and B, ricin, saporin, modeccin, nigrin, sarcin, concanavalin A, transcobalantin, catecholamines, transferrin, folate, riboflavin, vitamin B1, low density lipoprotein, maternal IgO, polymeric IgA, adenovirus, vesicular stomatitis virus, Rous sarcoma virus, V. cholerae, Kiebsiella strains, Serratia strains, parainfluenza virus, respiratory syncytial virus, Varicella zoster, and Enterobacter strains (see, for example, Swann, Pa., Pharmaceutical Research 15:826-832, 1998, incorporated herein by reference).

In certain additional embodiments, membrane-permeable peptides (for example, “arginine rich peptides”) are employed to facilitate delivery of biologically active agents. While the mechanism of action of these peptides remains to be fully elucidated, they provide useful delivery enhancing adjuncts for use within the compositions and methods herein. In one example, a basic peptide derived from human immunodeficiency virus (HIV)-1 Tat protein (for example, residues 48-60) has been reported to translocate effectively through cell membranes and accumulate in the nucleus, a characteristic which can be utilized for the delivery of exogenous proteins and peptides into cells. The sequence of Tat (GRKKRRQRRRPPQ, SEQ ID NO: 1) includes a highly basic and hydrophilic peptide, which contains 6 arginine and 2 lysine residues in its 13 amino acid residues. Various other arginine-rich peptides have been identified which have a translocation activity very similar to Tat-(48-60). These include such peptides as the D-amino acid- and arginine-substituted Tat-(48-60), the RNA-binding peptides derived from virus proteins, such as HIV-1 Rev, and flock house virus coat proteins, and the DNA binding segments of leucine zipper proteins, such as cancer-related proteins c-Fos and c-Jun, and the yeast transcription factor GCN4 (see, for example, Futaki et al., Journal Biological Chemistry 276:5836-5840, 2000, incorporated herein by reference). These peptides reportedly have several arginine residues marking their only identified common structural characteristic, suggesting a common internalization mechanism ubiquitous to arginine-rich peptides, which is not explained by typical endocytosis. Using (Arg)n (n=4-16) peptides, Futaki et al. teach optimization of arginine residues (n˜8) for efficient translocation. Recently, methods have been developed for the delivery of exogenous proteins into living cells with the help of arginine rich membrane-permeable carrier peptides such as HIV-1 Tat- and Antennapediasee, Futaki et al., supra, and references cited therein, incorporated herein by reference). By genetically or chemically hybridizing these carrier peptides with biologically active agents as described herein, additional methods and compositions are thus provided to enhance delivery.

It will be understood by those skilled in the art that while the compounds of the present disclosure will typically be employed as selective agonists or antagonists, there will be instances where a compound with a mixed steroid receptor profile is desired. For example, use of a PR agonists (for example, progestin) in female contraception often leads to the undesired effects of increased water retention and acne. In this instance, a compound that is primarily a PR agonist, but also displays some AR and MR modulating activity, can prove useful. Specifically, the mixed MR effects would be useful to control water balance in the body, while the AR effects would help to control any acne flare ups that occur.

Furthermore, it will be understood by those skilled in the art that the compounds of the present disclosure, including pharmaceutical compositions and formulations containing these compounds, can be used in a wide variety of combination therapies to treat various conditions and diseases as described herein. Thus, the compounds of the present disclosure can be used in combination with other active agents and other therapies, including, without limitation, chemotherapeutic agents such as cytostatic and cytotoxic agents, immunological modifiers such as interferons, interleukins, growth hormones and other cytokines, hormone therapies, surgery and radiation therapy.

A method of identifying a test agent that modulates LF blockade of the GR comprising: (a) obtaining cells that express the following: 1) GR; 2) an GR substrate, a GR reporter construct capable of measuring GR activity (such as GR pathway activation), or both a GR substrate and a GR reporter construct; (b) subjecting the cells to a test agent; (c) measuring the amount of GR activity, wherein activity of the GR is used to identify a test agent that modulates LF blockade of the GR. In one example, the ability of the agent to affect GR activity, but not to alter GR receptor number, identifies the agent as being of use.

The “glucocorticoid receptor” (GR) is a steroid hormone activated transcriptional factor known to regulate, either directly or indirectly, target genes involved in glucose homeostasis, bone turnover, cell differentiation, lung maturation, and inflammation (Reichardt et al., Adv. Pharmacol., 47:1-21, 2000). Mutations in GR are associated with Cushing's syndrome, autoimmune diseases, and various cancers (Warner et al., Steroids, 61: 216-221, 1996). As such, GR is widely recognized as a therapeutically important target. GR ligands, including dexamethasone, prednisolone, and other related corticosteroid analogs, are commonly used to treat diverse medical conditions such as asthma, allergic rhinitis, rheumatoid arthritis, and leukemia (Barnes et al., Am. J. Respir. Crit. Care Med., 157:S1-53, 1998). However, clinical use of oral corticosteroids is limited by a number of side effects ranging from increased bone loss and growth retardation to suppression of the hypothalamic-pituitary-adrenal axis. Discovery of a GR agonist that retains the beneficial anti-inflammatory activities without the undesired side effects is the subject of intense pharmaceutical efforts.

As noted above, GR belongs to the nuclear hormone receptor (NR) superfamily, which includes receptors for the mineralocorticoids (MR), estrogens (ER), progestins (PR), and androgens (AR), as well as receptors for peroxisome proliferators (PARs), vitamin D (VDR), and thyroid hormones (TR). Phylogenetic analysis and sequence alignments show that GR, MR, PR, and AR form a subfamily of oxosteroid receptors that are distinct from the ER subfamily (NRNC, 1999). These analysis are useful for evaluating structure-function relationships between GR and its cognate ligands and cofactors.

Like most nuclear hormone receptors, GR is a modular protein that is organized into three major domains: an N-terminal activation function-1 domain (AF-1), a central DNA binding domain (DBD), and a C-terminal ligand binding domain (LBD). In addition to its role in ligand recognition, the LBD contains a ligand-dependent activation function (AF-2) that is tightly regulated by hormone binding.

Within the context of the full-length receptor, both the Ar-1 function and the DNA binding activity of GR are dependent on hormone binding. In the absence of ligand, GR is retained in the cytoplasm by association with chaperone proteins such as hsp90 and p23, which bind to the LBD (Pratt et al., Endocr. Rev. 18:306-360, 1997). The chaperone activity of the hsp90 complex has been shown to be critical for hormone binding by GR (Bresnick et al., J. Biol. Chem., p. 4992-4997, 1989; Picard et al., Nature, 348:166-168, 1990). Hormone binding initiates the release of chaperone proteins from GR, allowing dimerization and translocation of the receptor into the nucleus. In the nucleus, GR binds to DNA promoter elements and can either activate or repress transcription depending on the context of the target promoters. In addition, GR can also crosstalk with other 110 transcriptional factors such as nuclear factor-κB (NF-κB) and activator protein-1 (AP-1) to repress their gene activation activities (reviewed in McKay et al., Endocr. Rev., 20:435-459, 1999). This GR mediated repression has been postulated to be a molecular basis for the anti-inflammatory and immunosuppressive activities of glucocorticoids. Both the ligand-dependent activation and repression by GR require the intact function of the LBD.

The molecular mechanism of ligand-dependent regulation of nuclear hormone receptors has been illustrated by crystal structures of more than a dozen NR LBDs that are either in the apo-state or bound to agonists or antagonists (Bourguet et al., Nature 375, 377-382 1995; Brzozowski et al., Nature 389, 753-758, 1997; Renaud et al., Nature 378, 681-689 1995; Wagner et al., Nature 378 690-697 1995; Xu et al., Nature 415, 813-817 1999). These analysis are also useful for evaluating structure-function relationships between GR and its cognate ligands and cofactors. The reported structures not only reveal that the LBDs fold into a canonical three-layer helical sandwich that embeds a hydrophobic pocket for ligand binding, but also highlight the importance of the C-terminal (AF-2) helix in ligand dependent regulation. In the apo- or antagonist-bound receptor, the AF-2 helix is destabilized from its “active” conformation to allow the LBD to interact with co-repressors such as nuclear co-repressor (N-CoR) and silencing mediator for retinoid and thyroid hormone receptors (SMRT; Clen and Evans, Nature 377:454-457, 1995; Horlein et al., Nature 377:397-404, 1995). Agonist binding induces a conformational change of the AF-2 helix, stabilizing the receptor in an active conformation to facilitate its association with coactivator proteins, such as steroid receptor coactivator-1 (SRC-1) and transcriptional intermediary factor 2 (TIF2; Onate et al., Science 270:1354-1357, 1995; Voegel et al., EMBO J. 17:507-519, 1996). These co-activators contain multiple LXXLL motifs, which interact with the NR LBD (Heery et al., Nature 387:733-736, 1997; Le Douarin et al., EMBO J. 15:6701-6715, 1996). Various crystal structures of receptor/co-activator peptide complexes have revealed a general mode of coactivator binding to NRs. In these structures, the coactivator LXXLL motifs adopt a two-tum a helix and both helical ends are stabilized by a “charge clamp” formed in part by a conserved acidic residue from the AF-2 helix (Dan′-mont et al., Genes Dev. 12:3343-3356, 1998; Nolte et al., Nature 395:137-143, 1998; Shiau et al., Cell 95:927-937, 1998).

Given its biological and pharmaceutical importance, there has been enormous interest in elucidating the GR LBD structure. However, these structural efforts have been hampered by the inability to obtain a purified receptor that retains ligand binding activity. In a recent report, die purification, crystallization, and structure determination of the GR LBD in complex with dexamethasone and a co-activator motif derived from the cofactor TIF2 is described (Bledsoe et al., Cell 110:93-105, 2002. Surprisingly, the structure reveals a novel dimer interface unlike that observed for any other nuclear hormone receptor. Mutagenesis studies support the importance of this dimer interface in GR function. The crystal structure also reveals an unanticipated second charge clamp that is responsible for the specificity for the third TIF2 LXXLL motif, and a distinct steroid binding pocket with features that explain ligand binding and selectivity. Since GR is highly homologous to MR, AR, and PR, the structure presented in this report serves as a model for understanding the roles of ligand binding, co-activator recruitment, and receptor dimerization in the signaling pathways mediated by these steroid receptors.

The glucocorticoid receptor is essential for survival and also for modulation of immune responses to infectious agents important in protecting against lethal effects of bacteria, such as septic shock. Loss of activity of the glucocorticoid receptor during infection could render the host more susceptible to the lethal or toxic effects of anthrax bacteria. Considering the mechanistic and therapeutic aspects disclosed herein, the findings herein indicate that simultaneous massive stimulation of cytokine release during anthrax infection, coupled with LF/LeTx repression of GR and other nuclear hormone receptors contribute to more severe consequences of infection including septic shock, increased stress and mortality, and exacerbated long-term sequelae due to the removal of the anti-inflammatory effects of the glucocorticoids released in response to infection. This scenario is consistent with the well-described increased mortality from septic shock in animals exposed to both glucocorticoid receptor antagonists and infectious agents or pro-inflammatory bacterial products. GR repression by LF/PA also likely contributes to the chronic fatigue syndrome-like symptoms, cognitive and inflammatory symptoms now being reported in relation to anthrax exposure, since blunted glucocorticoid responses have been associated with many inflammatory diseases, cognitive symptoms and fatigue states. Thus, in one embodiment, an agent that alters GR activity can be used to alter an immune response to an infectious agent.

Simultaneous loss of activity or enhancement of activity of other nuclear hormone receptors, including PR, and resulting imbalance in ratios of relative activity of nuclear hormone receptors likely amplifies these immune enhancing effects. Identification of nuclear hormone receptor co-factor interactions as a mechanism of toxicity of anthrax lethal factor and other bacterial products (such as bacterial toxins and antigens such as superantigens (SAgs) will therefore provide new tools and methods for treatment and prevention of the toxic effects of anthrax and other pathogenic infections. In more detailed aspects, these tools will be effective to minimize adverse side effects of infection, including toxicities, inflammatory symptoms, or related complications, including autoimmune diseases exacerbated by nuclear hormone receptor repression (for example, lupus, rheumatoid arthritis (RA), diabetes mellitus, multiple sclerosis, regional enteritis, thyroid cancer, and other diseases and conditions).

In addition, the methods and compositions disclosed herein provide tools for identification, removal and/or avoidance of host and or vaccine factors predisposing an individual to increased risk of adverse sequelae associated with pathogenic infection, inflammatory disorders and autoimmune disease. In the case of bacterial infection, products that repress nuclear hormone receptors are likely to account for idiopathic chronic fatigue syndromes, inflammatory arthritis and autoimmune diseases, and potentially for lethal and septic shock effects of certain bacterial strains. These products may also account for some ubiquitous idiopathic chronic inflammatory or fatigue symptoms unrelated to infectious exposures.

The disclosure concerning molecular interactions of the anthrax lethal factor with GR identify a novel mechanism by which the lethal toxin of Bacillus anthracis (anthrax LeTx), interferes with a number of nuclear hormone receptors essential for life and healthy functioning of cells. These findings have immediate important public health implications not only for anthrax infection and biodefense, but are also potentially relevant to explain toxicities related to a wide range of bacterial products and for the development of potential therapeutic interventions to prevent and treat toxic sequelae of infection with such pathogens.

In other embodiments, the selective and specific effects of LeTx and other bacterial products on range of nuclear hormone receptors make these products useful tools for elucidating the molecular mechanisms of interactions between bacterial products and nuclear hormone receptors and their co-factors.

Agents that affect the activity of a nuclear hormone receptor, as disclosed herein, are useful to influence basic, life sustaining systems of the body, including carbohydrate, protein and lipid metabolism, electrolyte and water balance, and the functions of the cardiovascular, kidney, central nervous, immune, skeletal muscle and other organ and tissue systems. In this regard, GR and MR modulators (agonists and antagonists) have proved useful in the treatment of inflammation, tissue rejection, auto-immunity, hypertension, various malignancies, such as luekerias, lymphomas and breast and prostate cancers, Cushing's syndrome, glaucoma, obesity, rheumatoid arthritis, acute adrenal insufficiency, congenital adrenal hyperplasia, osteoarthritis, rheumatic fever, systemic lupus erythematosus, polymyositis, polyarteritis nodosa, granulomatous polyarteritis, allergic diseases such as urticaria, drug reactions and hay fever, asthma, a variety of skin diseases, inflammatory bowel disease, hepatitis and cirrhosis. Accordingly, in some examples, GR and MR modulatory compounds are useful as immuno stimulants and repressors, wound healing and/or tissue repair agents, catabolic/antianabolic activators, and as antibacterial or anti-viral agents (such as for treatment or prevention of symptoms related to anthrax, herpes simplex viral infection and related symptoms).

The bacterial products that modulate nuclear hormone receptor activity (including naturally occurring, recombinant, and synthetic peptides and proteins, and peptide and protein analogs and mimetics of native bacterial products) can be used for screening (for example, in kits and/or screening assay methods) to identify additional compounds, including other peptides, proteins, analogs and mimetics, that will function within the methods and compositions disclosed herein, including as nuclear hormone receptor agonists and antagonists. Several methods of automating assays have been developed in recent years so as to permit screening of tens of thousands of compounds in a short period (see, for example, Fodor et al., Science 251:767-773, 1991, and U.S. Pat. Nos. 5,677,195; 5,885,837; 5,902,723; 6,027,880; 6,040,193; and 6,124,102, issued to Fodor et al., each incorporated herein by reference). Large combinatorial libraries of compounds can be constructed by encoded synthetic libraries (ESL) described in, for example, WO 95/12608, WO 93/06121, WO 94/08051, WO 95/35503, and WO 95/30642 (each incorporated by reference). Peptide libraries can also be generated by phage display methods (see, for example, Devlin, WO 91/18980, incorporated herein by reference). Many other publications describing chemical diversity libraries and screening methods are also considered reflective of the state of the art pertaining to these aspects and are generally incorporated herein.

One method of screening for agents that affect the activity of nuclear hormone receptors (such as to screen for small molecule drugs, LF analogs, and peptide mimetics that reduce or block LF or LeTx repression of GR or PR) utilizes eukaryotic or prokaryotic host cells which are stably transformed with recombinant DNA molecules expressing an active bacterial peptide or protein, for example, LF or LeTx. Such cells, either in viable or fixed form, can be used for standard assays, for example, ligand/receptor binding assays (see, for example, Parce et al., Science 246:243-247, 1989; and Owicki et al., Proc. Natl. Acad. Sci. USA 87:4007-4011, 1990, each incorporated herein by reference). Competitive assays are particularly useful, for example assays where the cells are contacted and incubated with a labeled receptor, receptor ligand, DNA binding target of the receptor, receptor cofactor, or antibody having binding affinity to the bacterial product or to an indirect binding partner that in turn binds the bacterial product. In conjunction with these assays, a test compound may be added to detect interruption of direct or indirect binding interactions. Bound and free labeled binding components are typically separated to assess the degree of specific binding and/or binding enhancement or inhibition. Any one of numerous techniques can be used to separate bound from free agents to assess the degree of binding (such as between a bacterial product and a cofactor of a nuclear hormone receptor, between a cofactor and its cognate receptor in the presence or absence of a selected bacterial toxin, etc.) This separation step can involve a conventional procedure such as adhesion to filters followed by washing, adhesion to plastic followed by washing, or centrifugation of the cell membranes.

Another technique for drug screening involves an approach which provides high throughput screening for compounds having suitable binding affinity to a target molecule, such as a labeled receptor, receptor ligand, DNA binding target of the receptor, receptor cofactor, or antibody having binding affinity to the bacterial product or to an indirect binding partner that in turn binds the bacterial product. Representative screening methods for use within these embodiments are provided, for example, in Geysen, European Patent Application 84/03564, published on Sep. 13, 1984 (incorporated herein by reference). First, large numbers of different test compounds, such as small peptides, are synthesized on a solid substrate, for example, plastic pins or some other appropriate surface, (see, for example, Fodor et al., Science 251:767-773, 1991, and U.S. Pat. Nos. 5,677,195; 5,885,837; 5,902,723; 6,027,880; 6,040,193; and 6,124,102, issued to Fodor et al., each incorporated herein by reference). Then all of the pins are reacted with a solubilized peptide agent, and washed. The next step involves detecting bound peptide.

Rational drug design may also be based upon structural studies of the molecular shapes of biologically active peptides and proteins determined to operate within the methods disclosed herein. Various methods are available and well known in the art for characterizing, mapping, translating, and reproducing structural features of peptides and proteins to guide the production and selection of new peptide mimetics, including for example x-ray crystallography and 2 dimensional NMR techniques. These and other methods, for example, will allow reasoned prediction of which amino acid residues present in a selected peptide or protein form molecular contact regions necessary for specificity and activity (see, for example, Blundell and Johnson, Protein Crystallography, Academic Press, N.Y., 1976, incorporated herein by reference).

Operable analogs and mimetics of bacterial products and of other biologically active agents disclosed herein retain partial, complete or enhanced activity compared to a native peptides, protein or unmodified compound. For example analogs or mimetics of LF or LeTx will exhibit partial or complete activity for nuclear hormone receptor repression. In this regard, operable analogs and mimetics for use will often retain at least 50%, often 75%, and up to 95-100% or greater levels of one or more selected activities as compared to the same activity observed for a selected native peptide or protein or unmodified compound. These biological properties of altered peptides or non-peptide mimetics can be determined according to any suitable assay disclosed or incorporated herein, for example by determining the ability of a LF peptide or mimetic to repress GR activation. Where bacterial products are contemplated for use as therapeutics, they will typically be engineered for reduced toxicity.

In accordance with the description herein, the compounds disclosed herein are useful in vitro as unique tools for analyzing the nature and function of interactions between bacterial products and members of nuclear hormone receptor activation and repression pathways. These compounds will therefore also serve as leads in various programs for designing additional peptide and non-peptide (for example, small molecule drug) agents for regulating activation and repression of nuclear hormone receptor activity, including in clinical contexts to treat or prevent disease and other conditions associated with aberrant functioning of one or more nuclear hormone receptors.

Those skilled in the art will readily appreciate that a wide range of additional screening assays can be employed to identify molecules capable of modulating one or more activities (such as ligand binding, DNA binding, expression of nuclear hormone receptor regulated endogenous genes or reporter constructs) of, for example, a bacterial product, nuclear hormone receptor, receptor ligand, DNA binding target of the receptor, or receptor cofactor. Such assays can involve the identification of compounds that interact with these and other compounds of interest, either physically (for example, by binding) or genetically, and can thus rely on any of a number of standard methods to detect physical or genetic interactions between multiple subject compounds. Such assays can also involve the identification of compounds that affect expression, activity or other properties, such as phosphorylation or nuclear localization, of the subject compound(s) or ability to bind yet additional binding partners, including labeled binding partners such as antibodies. Such assays can be cell-free or cell-based, and the latter type of assays can be performed in any type of cell, such as a cell that naturally or artificially incorporates or expresses one or more products of interest, for example one or more bacterial product(s), nuclear hormone receptor(s), receptor ligand(s), DNA binding target(s) of the receptor, receptor cofactor(s), etc.

Compounds that are involved in activation or repression of nuclear hormone receptor pathways can be identified and/or isolated based on an ability to specifically bind to a screening compound of interest, for example a bacterial product, nuclear hormone receptor, receptor ligand, DNA binding target of the receptor, or receptor cofactor. Likewise, screening methods for use can be based on binding to a fragment or conjugate of one of these subject compounds, or by binding to an antibody that likewise recognizes the subject compound. In numerous embodiments, the subject compound will be attached to a solid support. In one embodiment, affinity columns are made using the subject compound and physically-interacting molecules are identified. It will be apparent to one of skill that chromatographic techniques can be performed at any scale and using equipment from many different manufacturers. In addition, molecules that interact with subject compounds in vivo can be identified by co-immunoprecipitation or other methods, for example, immunoprecipitating subject bacterial proteins or cofactors using anti-antibodies to pull the subject compound(s) from a cell or cell extract, and identifying candidate compounds that bind the subject compounds that are precipitated along with the subject protein. Such methods are well known to those of skill in the art.

Two-hybrid screens can also be used to identify polypeptides that interact in vivo with a subject compound (see, for example, Fields et al., Nature 340:245-246, 1989). Such screens comprise two discrete, modular domains of a transcription factor protein, for example, a DNA binding domain and a transcriptional activation domain, which are produced in a cell as two separate polypeptides, each of which also comprises one of two potentially binding polypeptides. If the two potentially binding polypeptides (for example, a bacterial toxin and a cofactor of a nuclear hormone receptor) in fact interact in vivo, then the DNA binding and the transcriptional activating domain of the transcription factor are united, thereby producing expression of a target gene in the cell. The target gene typically encodes an easily detectable gene product, for example, β-galactosidase, GFP, or luciferase, which can be detected using standard methods. In one exemplary embodiment, a LF polypeptide is fused to one of the two domains of the transcription factor, and a known or potential nuclear hormone receptor cofactor polypeptide (for example, encoded by a cDNA library) is fused to the other domain. Such methods are well known to those of skill in the art.

In other embodiments, transcription levels can be measured to assess die effects of a test compound on nuclear hormone receptor pathway activity. In various examples, a host cell containing a nuclear hormone receptor of interest is transformed to express a “reporter construct” that yields a detectable signal for receptor pathway activity. Alternatively or in combination with this protocol, the cell may be contacted with, or genetically engineered to express, one or more of the following: a native or modified (such as a truncated mutant, chimeric, or tagged) receptor, receptor ligand, DNA binding target of the receptor, or a receptor cofactor, which are “substrates.” The cell is then exposed to a test compound for a sufficient time to effect any binding or other interactions between the test compound and subject compounds, and then the interactions are detected (such as by immunoprecipitation, detection of levels of gene expression, etc.) Levels of transcription may be measured using any method known to those of skill in the art to be suitable. For example, mRNA expression of a protein of interest may be detected using Northern blots or by detecting their polypeptide products using immunoassays. Many polynucleotides typically expressed following nuclear hormone receptor activation will thus be detectable. (see, for example, Lenardo, et al., Cell 58:227, 1989; Grilli, et al., Int. Rev. Cytol. 143:1, 1993; Baeuerle, et al., Ann. Rev. Immunol. 12:141, 1994. Such assays can use natural targets, for example targets of NF-κB or can use reporter genes, such as chloramphenicol acetyltransferase, luciferase, β-galactosidase, GFP, and alkaline phosphatase, operably linked to a promoter containing a binding site for a compound of interest (for example, a ligand of a nuclear hormone receptor). Furthermore, a protein of interest can be used as an indirect reporter via attachment to a second reporter such as green fluorescent protein (see, for example, Mistili & Spector, Nature Biotechnology 15:961-964, 1997.

The amount of transcription is then compared to the amount of transcription in either the same cell in the absence of the test compound, or it may be compared with the amount of transcription in a substantially identical cell that lacks one or more of the compound(s) interest (such as a cell that does not have an expression construct directing expression of a nuclear hormone receptor cofactor introduced into the test cell). A substantially identical cell may be derived from the same cells from which the recombinant cell was prepared but which had not been modified by introduction of heterologous DNA. Any difference in the amount of transcription indicates that the test compound has in some manner altered the activity of the protein of interest.

Compounds tested as modulators of nuclear hormone receptor activity can include any small chemical compound, or a biochemical compound such as a protein, peptide, protein, sugar, nucleic acid or lipid. Other test compounds will comprise a recombinantly or genetically modified nuclear hormone receptor, receptor ligand, DNA binding target of a receptor, receptor cofactor, or the like. Typically, test compounds will be small chemical molecules and peptides. Essentially any chemical compound can be used as a potential modulator or binding compound in the assays disclosed herein, although most often compounds that can be dissolved in aqueous or organic (especially DMSO-based) solutions are used. The assays are designed to screen large chemical libraries by automating the assay steps and providing compounds from any convenient source to assays, which are typically run in parallel (for example, in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In one embodiment, high throughput screening methods involve providing a combinatorial chemical or peptide library containing a large number of potential therapeutic compounds (potential modulator or binding compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A “combinatorial chemical library” is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (for example, the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, for example, U.S. Pat. No. 5,010,175; Furka, Int. J. Pept. Prot. Res., 37:487-493, 1991 and Houghton et al., Nature, 354:84-88, 1991). Other chemistries for generating chemical diversity libraries can also be used. Such chemistries include, but are not limited to: peptoids (for example, see PCT Publication No. WO 91/19735), encoded peptides (for example, PCT Publication No. WO 93/20242), random bio-oligomers (for example, see PCT Publication No. WO 92/00091), benzodiazepines (for example, see U.S. Pat. No. 5,288,514), diversomers such as hydantoins, benzodiazepines and dipeptides (Hobbs et al., Proc. Nat. Acad. Sci. USA 90, 6909-6913, 1993), vinylogous polypeptides (Hagihara et al., J. Amer. Chem. Soc., 114:65-68, 1992), nonpeptidal peptidomimetics with glucose scaffolding (Hirschmann et al., J. Amer. Chem. Soc., 114:9217-9218, 1992), analogous organic syntheses of small compound libraries (Chen et al., J. Amer. Chem. Soc. 116:2661, 1994), oligocarbamates (Cho et al., Science, 61:1303, 1993), and/or peptidyl phosphonates (Campbell et al., J. Org. Chem., 59:658, 1994), nucleic acid libraries and peptide nucleic acid libraries (for example, see U.S. Pat. No. 5,539,083), antibody libraries (see, for example, Vaughn et al., Nature Biotechnology, 14:309-314, 1996 and PCT/US96/10287), carbohydrate libraries (for example, see Liang et al., Science, 274:1520-1522, 1996 and U.S. Pat. No. 5,593,853), small organic molecule libraries for example, benzodiazepines, Baum C&EN, January 18, page 33 (1993); isoprenoids, U.S. Pat. No. 5,569,588; thiazolidinones and metatliazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like.

Devices for the preparation of combinatorial libraries are commercially available (see, for example, 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, for example, ComGenex, Princeton, N.J., Tripos, Inc., St. Louis, Mo., 3D Pharmaceuticals, Exton, Pa., Martek Biosciences, Columbia, Md., etc.)

In the high throughput assays disclosed herein, it is possible to screen up to several thousand different subject compounds in a single day. In particular, each wen of a microtiter plate can be used to run a separate assay against a selected potential nuclear hormone receptor modulator, or, if concentration or incubation time effects are to be observed, every 5-10 wells can test a single modulator. Thus, a single standard microtiter plate can assay about 100 (for example, 96) modulators. If 1536 well plates are used, then a single plate can easily assay from about 100 to about 1500 different compounds. It is possible to assay several different plates per day; assay screens for up to about 6,000-20,000 different compounds is possible using the integrated systems disclosed herein. More recently, microfluidic approaches to reagent manipulation have been developed.

The molecule of interest can be bound to a solid state component, directly or indirectly, via covalent or non covalent linkage, such as via a tag. The tag can be any of a variety of components.

In general, a molecule which binds the tag (a tag binder) is fixed to a solid support, and the tagged molecule of interest is attached to the solid support by interaction of the tag and the tag binder. A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Inmaunochemicals 1998 catalogue SIGMA, St. Louis Mo.). Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure. Common linkers such as peptides, polyethers, and the like can also serve as tags, and include polypeptide sequences, such as poly-gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalylsilanes can be used to functionalize a variety of surfaces, such as glass surfaces. The construction of such solid phase biopolymer arrays is well described in the literature. See, for example, Merrifield, J. Am. Chem. Soc., 85:2149-2154, 1963, (describing solid phase synthesis of, for example, peptides); Geysen et al., J. Immun. Meth., 102:259-274, 1987 (describing synthesis of solid phase components on pins); Frank et al., Tetrahedron 44:6031-6040, 1988 (describing synthesis of various peptide sequences on cellulose disks); Fodor et al., Science, 251:767-777, 1991; Sheldon, et al., Clinical Chemistry, 39:718-719, 1993; and Kozal et al., Nature Medicine, 2:753-759, 1996 (all describing arrays of biopolymers fixed to solid substrates). Nonchemical approaches for fixing tag binders to substrates include other common methods, such as heat, cross-linking by UV radiation, and the like.

Yet another assay for compounds that modulate nuclear hormone receptor activity involves computer assisted drug design, in which a computer system is used to generate a three-dimensional structure of, for example, a bacterial product, nuclear hormone receptor, receptor ligand, or receptor cofactor based, for example on the structural information encoded by its amino acid sequence. The input amino acid sequence interacts directly and actively with a preestablished algorithm in a computer program to yield secondary, tertiary, and quaternary structural models of the protein. The models of the protein structure are then examined to identify regions of the structure that have the ability to bind. These regions are then used to identify compounds that bind to the protein. The three-dimensional structural model of the protein is generated by entering protein amino acid sequences of at least 10 amino acid residues or corresponding nucleic acid sequences encoding a subject polypeptide into the computer system. The nucleotide sequence encoding the polypeptide may, for example, comprise a sequence encoding a portion of a bacterial product or nuclear hormone receptor cofactor, or a conservatively modified version thereof. At least 10 residues of the amino acid sequence (or a nucleotide sequence encoding 10 amino acids) are entered into the computer system from computer keyboards, computer readable substrates that include, but are not limited to, electronic storage media (for example, magnetic diskettes, tapes, cartridges, and chips), optical media (for example, CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, for example, cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential modulator binding regions are identified by the computer system. Three-dimensional structures for potential modulators are generated by entering amino acid or nucleotide sequences or chemical formulas of compounds, as described above. The three-dimensional structure of the potential modulator is then compared to that of the subject protein to identify compounds likely to bind to the protein. Binding affinity between the protein and compound is determined using energy terms to determine which compounds have an enhanced probability of binding to the protein.

In numerous embodiments, a compound, for example, nucleic acid, polypeptide, or other molecule is administered to a patient, in vivo or ex vivo, to effect a change in nuclear hormone receptor activity or expression in the patient. Such compounds can include nucleic acids encoding any of the compounds of interest identified herein or selected according to the screening methods disclosed herein (as well as recombinantly modified derivatives, fragments, variants, or fusions thereof), operably linked to a promoter. Suitable nucleic acids also include inhibitory sequences such as antisense or ribozyme sequences, which can be delivered in, for example, an expression vector operably linked to a promoter, or can be delivered directly. Also, any nucleic acid that encodes a polypeptide that modulates the expression of a nuclear hormone receptor can be used. In general, nucleic acids can be delivered to cells using any of a large number of vectors or methods, for example, retroviral, adenoviral, or adeno-associated virus vectors, liposomal formulations, naked DNA injection, and others. All of these methods are well known to those of skill in the art.

The therapeutic compounds, for example native or modified bacterial products, nuclear hormone receptors, receptor cofactors, and antibodies having binding affinity to a bacterial product or cofactor, are generally provided for direct administration to subjects in a substantially purified form. The term “substantially purified” as used herein, is intended to refer to a peptide, protein, nucleic acid or other compound that is isolated in whole or in part from naturally associated proteins and other contaminants, wherein the peptide, protein, nucleic acid or other active compound is purified to a measurable degree relative to its naturally-occurring state, for example, relative to its purity within a cell extract.

In certain embodiments, the term “substantially purified” refers to a peptide, protein, or polynucleotide composition that has been isolated from a cell, cell culture medium, or other crude preparation and subjected to fractionation to remove various components of the initial preparation, such as proteins, cellular debris, and other components. Of course, such purified preparations may include materials in covalent association with the active agent, such as glycoside residues or materials admixed or conjugated with the active agent, which may be desired to yield a modified derivative or analog of the active agent or produce a combinatorial therapeutic formulation, conjugate, fusion protein or the like. The term purified thus includes such desired products as peptide and protein analogs or mimetics or other biologically active compounds wherein additional compounds or moieties such as polyethylene glycol, biotin or other moieties are bound to the active agent in order to allow for the attachment of other compounds and/or provide for formulations useful in therapeutic treatment or diagnostic procedures.

As applied to polynucleotides, the term substantially purified denotes that the polynucleotide is free of substances normally accompanying it, but may include additional sequence at the 5′ and/or 3′ end of the coding sequence which might result, for example, from reverse transcription of the noncoding portions of a message when the DNA is derived from a cDNA library, or might include the reverse transcript for the signal sequence as well as the mature protein encoding sequence.

When referring to peptides, proteins and peptide analogs (including peptide fusions with other peptides and/or proteins) of use, the term substantially purified typically means a composition which is partially to completely free of other cellular components with which the peptides, proteins or analogs are associated in a non-purified, for example, native state or environment. Purified peptides and proteins are generally in a homogeneous or nearly homogenous state although it can be either in a dry state or in an aqueous solution. Purity and homogeneity are typically determined using analytical chemistry techniques such as polyacrylamide gel electrophoresis or high performance liquid chromatography.

Generally, substantially purified peptides, proteins and other active compounds for use comprise more than 80% of all macromolecular species present in a preparation prior to admixture or formulation of the peptide, protein or other active agent with a pharmaceutical carrier, excipient, buffer, absorption enhancing agent, stabilizer, preservative, adjuvant or other co-ingredient in a complete pharmaceutical formulation for therapeutic administration. More typically, the peptide or other active agent is purified to represent greater than 90%, often greater than 95% of all macromolecular species present in a purified preparation prior to admixture with other formulation ingredients. In other cases, the purified preparation of active agent may be essentially homogeneous, wherein other macromolecular species are not detectable by conventional techniques.

Therapeutic and prophylactic formulations can include a biologically active subject compound as described above typically combined together with one or more pharmaceutically acceptable carriers and, optionally, other therapeutic ingredients. The carrier(s) must be “pharmaceutically acceptable” in the sense of being compatible with the other ingredients of the formulation and not eliciting an unacceptable deleterious effect in the subject. Such carriers are described herein above or are otherwise well known to those skilled in the art of pharmacology. Desirably, the formulation should not include substances such as enzymes or oxidizing agents with which the biologically active agent to be administered is known to be incompatible. The formulations may be prepared by any of the methods well known in the art of pharmacy.

Within the compositions and methods disclosed herein, the active subject compound (including peptides, proteins, analogs and mimetics, and other biologically active agents disclosed herein) may be administered to subjects by a variety of mucosal administration modes, including by oral, rectal, intranasal, intrapulmonary, or transdermal delivery, or by topical delivery to other surfaces. Optionally, the active agents disclosed herein can be administered by non-mucosal routes, including by intramuscular, subcutaneous, intravenous, intra-atrial, intra-articular, intraperitoneal, or parenteral routes. In other alternative embodiments, the biologically active agent(s) can be administered ex vivo by direct exposure to cells, tissues or organs originating from a mammalian subject, for example as a component of an ex vivo tissue or organ treatment formulation that contains the biologically active agent in a suitable, liquid or solid carrier.

To formulate pharmaceutical compositions, the biologically active agent can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (for example, benzyl alcohol), isotonizing agents (for example, sodium chloride, mannitol sorbitol), adsorption inhibitors (for example, Tween 80), solubility enhancing agents (for example, cyclodextrins and derivatives thereof), stabilizers (for example, serum albumin), and reducing agents (for example, glutathione) can be included. When the composition for delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about ⅓ to 3, more typically ½ to 2, and most often ¾ to 1.7.

The biologically active agent can be dispersed in a base or vehicle, which may comprise a hydrophilic compound having a capacity to disperse the active agent and any desired additives. The base can be selected from a wide range of suitable carriers, including but not limited to, copolymers of polycarboxylic acids or salts thereof, carboxylic anhydrides (for example, maleic anhydride) with other monomers (for example, methyl (meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc. can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the biologically active agent.

The biologically active agent can be combined with the base or carrier according to a variety of methods, and release of the active agent may be by diffusion, disintegration of the carrier, or associated formulation of water channels. In some circumstances, the active agent is dispersed in microcapsules (microspheres) or nanocapsules (nanospheres) prepared from a suitable polymer, for example, isobutyl 2-cyanoacrylate (see, for example, Michael et al., J. Pharmacy Pharmacol. 43: 1-5, 1991), and dispersed in a biocompatible dispersing medium, which yields sustained delivery and biological activity over a protracted time.

The compositions can alternatively contain as pharmaceutically acceptable carriers substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

Therapeutic compositions for administering the biologically active agent(s) can also be formulated as a solution, microemulsion, or other ordered structure suitable for high concentration of active ingredients. The carrier can be a solvent or dispersion medium containing, for example, water, ethanol, polyol (for example, glycerol, propylene glycol, and liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols such as mannitol, sorbitol, or sodium chloride in the composition. Prolonged absorption of the biologically active agent can be brought about by including in the composition an agent which delays absorption, for example, monostearate salts and gelatin.

In certain embodiments, the biologically active agent is administered in a time release formulation, for example in a composition which includes a slow release polymer. The active agent can be prepared with carriers that will protect against rapid release, for example a controlled release vehicle such as a polymer, microencapsulated delivery system or bioadhesive gel. Prolonged delivery of the active agent, in various compositions disclosed herein, can be brought about by including in the composition agents that delay absorption, for example, aluminum monosterate hydrogels and gelatin. When controlled release formulations of the biologically active agent is desired, controlled release binders suitable for use include any biocompatible controlled-release material which is inert to the active agent and which is capable of incorporating the biologically active agent. Numerous such materials are known in the art. Useful controlled-release binders are materials that are metabolized slowly under physiological conditions following their delivery. Appropriate binders include but are not limited to biocompatible polymers and copolymers previously used in the art in sustained release formulations. Such biocompatible compounds are non-toxic and inert to surrounding tissues, and do not trigger significant adverse side effects such as nasal irritation, immune response, inflammation, or the like. They are metabolized into metabolic products that are also biocompatible and easily eliminated from the body.

Exemplary polymeric materials for use in this context include, but are not limited to, polymeric matrices derived from copolymeric and homopolymeric polyesters having hydrolysable ester linkages. A number of these are known in the art to be biodegradable and to lead to degradation products having no or low toxicity. Exemplary polymers include polyglycolic acids (PGA) and polylactic acids (PLA), poly(DL-lactic acid-co-glycolic acid)(DL PLGA), poly(D-lactic acid-coglycolic acid)(D PLGA) and poly(L-lactic acid-co-glycolic acid)(L PLGA). Other useful biodegradable or bioerodable polymers include but are not limited to such polymers as poly(epsilon-caprolactone), poly(epsilon-aprolactone-CO-lactic acid), poly(epsilon.-aprolactone-CO-glycolic acid), poly(beta-hydroxy butyric acid), poly(alkyl-2-cyanoacrilate), hydrogels such as poly(hydroxyethyl methacrylate), polyamides, poly(amino acids) (for example, L-leucine, glutamic acid, L-aspartic acid and the like), poly (ester urea), poly (2-hydroxyethyl DL-aspartamide), polyacetal polymers, polyorthoesters, polycarbonate, polymaleamides, polysaccharides and copolymers thereof. Many methods for preparing such formulations are generally known to those skilled in the art (see, for example, Sustained and Controlled Release Drug Delivery Systems, J. R. Robinson, ed., Marcel Dekker, Inc., New York, 1978, incorporated herein by reference). Other useful formulations include controlled-release compositions such as are known in the art for the administration of leuprolide (trade name: Lupron.RTM.), for example, microcapsules (U.S. Pat. Nos. 4,652,441 and 4,917,893, each incorporated herein by reference), lactic acid-glycolic acid copolymers useful in making microcapsules and other formulations (U.S. Pat. Nos. 4,677,191 and 4,728,721, each incorporated herein by reference), and sustained-release compositions for water-soluble peptides (U.S. Pat. No. 4,675,189, incorporated herein by reference).

The pharmaceutical formulations typically must be sterile and stable under all conditions of manufacture, storage and use. Sterile solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders, methods of preparation include vacuum drying and freeze-drying which yields a powder of the active ingredient plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

In more detailed aspects, the biologically active agent is stabilized to extend its effective half-life following delivery to the subject, particularly for extending metabolic persistence in an active state within the physiological environment (for example, at a mucosal surface, in the bloodstream, or within a connective tissue compartment or fluid-filled body cavity). For this purpose, the biologically active agent may be modified by chemical means, for example, chemical conjugation, N-terminal capping, PEGylation, or recombinant means, for example, site-directed mutagenesis or construction of fusion proteins, or formulated with various stabilizing agents or carriers. Thus stabilized, the active agent administered as above retains biological activity for an extended period (for example, 2-3, up to 5-10 fold greater stability) under physiological conditions compared to its non-stabilized form.

In accordance with the various treatment methods disclosed herein, the biologically active agent is delivered to a mammalian subject in a manner consistent with conventional methodologies associated with management of the disorder for which treatment or prevention is sought. In accordance with the disclosure herein, a prophylactically or therapeutically effective amount of the biologically active agent is administered to a subject in need of such treatment for a time and under conditions sufficient to prevent, inhibit, and/or ameliorate a selected disease or condition or one or more symptom(s) thereof.

The term “subject” as used herein means any mammalian patient to which the compositions can be administered. Typical subjects intended for treatment with the compositions and methods disclosed herein include humans, as well as non-human primates and other animals. To identify subject patients for prophylaxis or treatment according to the methods disclosed herein, accepted screening methods are employed to determine risk factors associated with a targeted or suspected disease of condition as discussed above, or to determine the status of an existing disease or condition in a subject. These screening methods include, for example, conventional work-ups to determine familial, sexual, drug-use and other such risk factors that may be associated with the targeted or suspected disease or condition, as well as diagnostic methods such as various ELISA immunoassay methods, which are available and well known in the art to detect and/or characterize disease-associated markers. These and other routine methods allow the clinician to select patients in need of therapy using the methods and formulations disclosed herein. In accordance with these methods and principles, biologically active agents may be administered according to the teachings herein as an independent prophylaxis or treatment program, or as a follow-up, adjunct or coordinate treatment regimen to other treatments, including surgery, vaccination, immunotherapy, hormone treatment, cell, tissue, or organ transplants, and the like.

For prophylactic and treatment purposes, the biologically active agent(s) disclosed herein may be administered to the subject in a single bolus delivery, via continuous delivery (for example, continuous transdermal, mucosal, or intravenous delivery) over an extended time period, or in a repeated administration protocol (for example, by an hourly, daily or weekly, repeated administration protocol). In this context, a therapeutically effective dosage of the biologically active agent(s) may include repeated doses within a prolonged prophylaxis or treatment regimen, that will yield clinically significant results to alleviate one or more symptoms or detectable conditions associated with a targeted disease or condition as set forth above. Determination of effective dosages in this context is typically based on animal model studies followed up by human clinical trials and is guided by determining effective dosages and administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (for example, immunologic and histopathologic assays). Using such models, only ordinary calculations and adjustments are typically required to determine an appropriate concentration and dose to administer a therapeutically effective amount of the biologically active agent(s) (for example, amounts that are intranasally effective, transdermally effective, intravenously effective, or intramuscularly effective to elicit a desired response). In alternative embodiments, an “effective amount” or “effective dose” of the biologically active agent(s) may simply inhibit or enhance one or more selected biological activity(ies) correlated with a disease or condition, as set forth above, for either therapeutic or diagnostic purposes.

The actual dosage of biologically active agents will of course vary according to factors such as the disease indication and particular status of the subject (for example, the subject's age, size, fitness, extent of symptoms, susceptibility factors, etc), time and route of administration, other drugs or treatments being administered concurrently, as well as the specific pharmacology of the biologically active agent(s) for eliciting the desired activity or biological response in the subject. Dosage regimens may be adjusted to provide an optimum prophylactic or therapeutic response. A therapeutically effective amount is also one in which any toxic or detrimental side effects of the biologically active agent is outweighed in clinical terms by therapeutically beneficial effects. A non-limiting range for a therapeutically effective amount of a biologically active agent within the methods and formulations disclosed herein is 0.01 μg/kg-10 mg/kg, more typically between about 0.05 and 5 mg/kg, and in certain embodiments between about 0.2 and 2 mg/kg. Dosages within this range can be achieved by single or multiple administrations, including, for example, multiple administrations per day, daily or weekly administrations. Per administration, it is desirable to administer at least one microgram of the biologically active agent, more typically between about 10 μg and 5.0 mg, and in certain embodiments between about 100 μg and 1.0 or 2.0 mg to an average human subject. It is to be further noted that for each particular subject, specific dosage regimens should be evaluated and adjusted over time according to the individual need and professional judgment of the person administering or supervising the administration of the permeabilizing peptide(s) and other biologically active agent(s).

Dosage of biologically active agents may be varied by the attending clinician to maintain a desired concentration at the target site. For example, a selected local concentration of the biologically active agent in the bloodstream or CNS may be about 1-50 nanomoles per liter, sometimes between about 1.0 nanomole per liter and 10, 15 or 25 nanomoles per liter, depending on the subject's status and projected or measured response. Higher or lower concentrations may be selected based on the mode of delivery, for example, trans-epidermal, rectal, oral, or intranasal delivery versus intravenous or subcutaneous delivery. Dosage should also be adjusted based on the release rate of the administered formulation, for example, of an intrapulmonary spray versus powder, sustained release oral versus injected particulate or transdermal delivery formulations, etc. To achieve the same serum concentration level, for example, slow-release particles with a release rate of 5 nanomolar (under standard conditions) would be administered at about twice the dosage of particles with a release rate of 10 nanomolar. Additional guidance as to particular dosages for selected biologically active agents for use can be found widely disseminated in the literature.

Kits, packages and multicontainer units containing the above described pharmaceutical compositions, active ingredients, and/or means for administering the same for use in the prevention and treatment of diseases and other conditions in mammalian subjects are disclosed herein. Briefly, these kits include a container or formulation that contains one or more of the biologically active subject compounds described above formulated in a pharmaceutical preparation for administration to a mammalian subject. The biologically active agent(s) is/are optionally contained in a bulk dispensing container or unit or multi-unit dosage form. Optional dispensing means can be provided, for example a pulmonary or intranasal spray applicator. Packaging materials optionally include a label or instruction indicating for what treatment purposes and/or in what manner the pharmaceutical agent packaged therewith can be used.

The following examples are provided by way of illustration, not limitation. These examples show that an exemplary bacterial product, Anthrax lethal toxin (LeTx) represses transactivation of the well-nown nuclear hormone receptor GR in a transient transfection system, and also represses activity of an endogenous GR-regulated gene. This repression is non-competitive and does not affect ligand binding or DNA binding, indicating that LeTx exerts its effects indirectly, presumptively through a cofactor(s) involved in the interaction between GR and the basal transcription machinery. LeTx-nuclear hormone receptor repression is partially selective, repressing GR, and two other nuclear hormone receptors, progesterone receptor B (PR-B) and estrogen receptor a (Era), but not the mineralocorticoid receptor (M) or ERβ. Simultaneous loss of GR and other nuclear hormone receptor activities could render the host more susceptible to lethal or toxic effects of anthrax infection by removing the normally protective anti-inflammatory effects of these hormones, similar to the increased mortality from septic shock seen in animals exposed to both GR antagonists and infectious agents or bacterial products. Accordingly, the present disclosure evinces for the first time that a bacterial product acts alters the activity of hormone receptor. This decreased activity substantially accounts for shock and other adverse sequelae associated with bacterial infection in mammalian subjects. More specifically, by blocking GR in the context of host exposure to anthrax bacterial products, LeTx impairs the anti-inflammatory protective effects of glucocorticoids released during infection—in much the same manner as GR antagonists act in relatively inflammatory-resistant rodents exposed to other bacterial products.

This surprising identification of nuclear hormone receptor co-factor interactions as a mechanism of toxicity of anthrax lethal factor provides for development of new treatments and prevention of the toxic effects of anthrax and for novel methods and compositions to provide new and more effective tools for modulating nuclear hormone receptor activity and diseases and other conditions mediated by diminished or excessive levels or activity of nuclear hormone receptors and/or their cognate ligands and cofactors.

### EXAMPLES

### Example 1

### General methods

The mechanisms of action of LF inside the cell were poorly understood prior to the present disclosure. LF is a metalloprotease that cleaves the MAP kinase kinases (MAPKK), including MEK1, MEK2, MKK3, MKK4, MKK6 and MKK7 but not MEK5 (K. R. Klimpel et al., Mol. Microbiol., 13:1093, 1994; N. S. Duesbery et al., Science, 280:734, 1998; R. Pellizzari et al., FEBS lett., 462:199, 1999; R. Pellizzari et al., Int. J. Med. Microbiol., 290:421, 2000; G. Vitale et al., Biochem. J., 352,-:739, 2000), thereby inhibiting the MAPK pathway. However, the fact that LeTx resistant and sensitive cells show similar internalization of LF (Y. Singh et al., J. Biol. Chem., 264:11099, 1989), and similar MPK degradation in response to LF (R Pellizzari et al., FEBS lett., 462:199, 1999; R. Pellizzari, Int. J. Med. Microbiol., 290:421, 2000), indicates that these factors cannot alone account for differential susceptibility or resistance to the toxin. Other factors that have been proposed to play a role in toxicity of LeTx include the proteosome (G. Tang et al., Infect. Immun., 67:3055, 1999), intracellular calcium stores (S. Shin et al., Cell. Biol. Toxicol., 16:137, 2000; R. Bhatnagar et al., Infect. Immun., 57,:2107, 1989), calmodulin (R. Bhatnagar et al., Infect. Immun., 57:2107, 1989), a calyculin A sensitive protein phosphatase (J. H. Kau et al., Curr. Microbiol., 44:106, 2002), protein synthesis (R. Bhatnagar et al., Infect. Immun., 62:2958, 1994) and reactive oxygen intermediates (P. C. Hanna et al., Mol. Med., 1:7, 1994). It is not known which of these or other unknown factors contribute to the well-described differential cell line and rodent strain sensitivities to toxic effects of LeTx. Recently, the gene Kif1C has been determined to be different between resistant and sensitive strains although the implication of this is not understood (J. W. Watters et al., Curr. Biol., 11:1503, 2001; J. E. Roberts et al., Mol. Microbiol., 29:581, 1998).

Fischer (F344/N) rats have long been known to be particularly susceptible to the LeTx (F. Klein et al., J. Bacteriol., 85:1032, 1963), with death occurring within 40 minutes after exposure to a lethal dose (J. W. Ezzell et al., Infect. Immun., 45:761, 1984). F344/N rats are also known to be relatively inflammatory disease resistant, due in part to their hypothalamic-pituitary-adrenal (HPA) axis hyper-responsiveness and resultant hyper-secretion of glucocorticoids from the adrenal glands in response to pro-inflammmatory and other stimuli. Similar to F344/N rats, BALB/c mice have a hyper-responsive HPA axis (N. Shanks et al., Pharmacol. Biochem. Behav., 36:515, 1990) and are also susceptible to LeTx (S. L. Welklos et al., Infect Immun., 51:795, 1986). Ordinarily this hyper-HPA axis responsiveness protects against inflammatory and autoimmune diseases through the anti-inflammatory and immunosuppressive effects of the glucocorticoids. However, F344/N rats and other inflammatory resistant rodent strains become highly susceptible to inflammation and rapid death from septic shock after simultaneous glucocorticoid receptor (GR) or HPA axis blockade and exposure to pro-inflammatory or infectious stimuli, including bacterial products such as streptococcal cell walls (SCW) or bacterial lipopolysaccharide (LPS) (C. K. I. Edwards et al., Proc. Natl. Acad. Sci. U.S.A., 88:2274, 1991; S. H. Zuckerman et al., Infect. Immun., 60:2581, 1992; E. M. Sternberg et al., Proc. Natl. Acad. Sci. U.S.A., 86:2374, 1989; M. C. Ruzek et al., J. Immunol., 162:3527, 1999; I. A. M. MacPhee et al., J. Exp. Med., 169:431, 1989).

Cell Culture

Cos7, HTC, J774.1, Raw264.7. IC-21 and MT2 cells were grown at 37° C. and 5% CO₂ in Dulbecco's modified Eagle's medium (DMEM) containing 10% serum, 10 mg/ml penicillin-streptomycin and 2 mM glutamine.

Transient Transfections

Cos7 cells were plated in 24-well plates at a density of 5×10⁵ cells/well in DMEM containing 10% charcoal-stripped serum, 10 mg/ml penicillin-streptomycin and 2 mM glutamine one day prior to transfection. Cos7 cells were transfected overnight with 20 ng receptor expression plasmid (SVGR, ERα, ERβ, MR or PR-B), 100 ng reporter construct ((GRE)₂-TK luc, ERE-luc, pLTR-luc, or pGL3 control), 60 ng pSG5 (Stratagene) and 20 ng PRL TK (Promega, constitutive renilla luciferase control) using Fugene6 (Roche) according to manufacturer's instructions. The medium was then replaced with DMEM containing 10% charcoal-stripped serum, the appropriate hormone and LF and/or PA or inhibitor as required. After 24 hr the cells were lysed and the firefly and renilla luciferases assayed using the dual luciferase assay (Promega).

Assay of Tyrosine Aminotransferase (TA 17 in HTC Cells

HTC cells were plated in 6 cm plates at a density of 5×10⁶ cells/plate in DMEM containing 10% fetal calf serum, 10 mg/ml penicillin-streptomycin and 2 mM glutamine one day prior to treatment. The media was then replaced with DMEM containing increasing concentrations of dexamethasone (Dex) either alone or together with lethal factor (LF) and protective antigen (PA). After 18 hours the cells were lysed by sonication and tyrosine aminotransferase (TAT) activity assayed as described by Thompson et al. (Proc. Natl. Adac. Sci. U.S.A. (1966) 56, 296-303).

Animal Experiments

Male and female BALB/cJ mice (10-12 weeks old, Jackson Laboratories, Bar Harbor, Me.) were injected intraperitoneally (IP) with 50 mg LF, 50 mg PA, or a combination of both in 1 ml sterile-filtered phosphate buffered saline (PBS) 30 minutes prior to Dex treatment. Dex was injected IP in 0.25 ml volume (0.06 mg/mouse). Mice were euthanized by CO₂ at various times post-injection, and livers were removed, homogenized in ice-cold lysis buffer (0.2 mM pyridoxal phosphate, 0.5 mM α-keto glutarate, 0.1 M potassium phosphate, pH 7.6, and then centrifuged at 100,000×g at 4° C. for 30 minutes. TAT activity of supernatants was assayed as described by Thompson et al. (Proc. Natl. Acad. Sci. U.S.A. (1966) 56, 296-303).

Western Blot Analysis

Cos7 cells were plated in 6-cm plates at a density of 5×10⁶ cells/plate two days prior to treatments. Cells were treated with MAP kinase inhibitors for 30 minutes. Cells were stimulated by addition of 10 mg/ml lipopolysaccharide (LPS) or anisomycin for 30 minutes. Proteins were solubilized using M-PER (Pierce) and 10 mg separated by sodium dodecyl sulphate polyacrylamide-gel electrophoresis (SDS-PAGE) according to the method of Laemmli (Nature (1970) 227, 680-685). Proteins were transferred to Polyvinylidene Fluoride (PVDF) and probed with antibodies against phospho-p38 MAP kinase (Thr180/Tyr182), phospho-p44/42 MAP kinase (Thr202/Tyr204) and phospho-c-Jun (Ser63) (Cell Signaling Technology). Chemiluminescence was detected and analyzed using the Chemidoc gel imaging system and volume analysis tool of the Quantity One software (Biorad).

Cytosol Prep of GR Transfected Cos7Cells

Cos7 cells were plated in 10 cm plates at a density of 1×10⁷ cells/plate in DMEM containing 10% serum, 10 mg/ml penicillin-streptomycin and 2 mM glutamine one day prior to transfection. Cos7 cells were transfected with 2 μg SV glucocorticoid receptor (SVGR) expression plasmid using Fugene6 (Roche) according to manufacturer's instructions. After 48 hours, cell cytosol was prepared by washing the cells in ice-cold PBS and then re-suspending them in ice-cold EPGMo buffer (1 mM EDTA, 20 mM potassium phosphate pH 7.8, 10% glycerol, 20 mM sodium molybdate and 1 mM DTT). The re-suspended cells were allowed to sit on ice for 10 minutes and then homogenized using a glass homogenizer 30 times on ice. The broken cells were then centrifuged at 100,000×g for 3 minutes at 4° C. to pellet the cell membranes. The protein content of the supernatant containing the cytosol was assayed.

Gel Shift

GR gel shift oligonucleotides (Santa Cruz Biotechnology, Inc) were allowed to anneal in a buffer containing 50 mM tris-HCl (pH 7.5-7.8), 10 mM MgCl₂ and 0.1 M NaCl by heating at 65° C. for 5 minutes and then cooling slowly to room temperature. The annealed probe was then radio-labeled with [γ-³²P]adenosine triphosphate (ATP) by incubation at 37° C. for 30 minutes with T4 polynucleotide kinase (USB). The probe was then cleaned using a P-6 micro Bio-Spin chromatography column (Biorad) and re-suspended at a concentration of 0.5 ng/μl. The specific activity of the probe was calculated.

The binding reaction was carried out in a buffer containing 20 mM 42-hydroxyethyl)-1-piperazineethanesulfonic acid (HEPES) pH 7.9, 20% glycerol, 100 mM KCl and 0.2 mM ethylenediaminetetraacetic acid (EDTA) with poly dI-dC, cytosol preparation, probe and competitor or LF and/or PA as required for 30 minutes at room temperature. A 40% (weight to volume) 29:1 acrylamide/bisacrylamide Tris-Borate-EDTA (TBE) gel was pre-run in 0.5×TBE for about 20 minutes at 200 Volts. Two μl loading dye was added to each sample before loading onto the gel. The gel was run at 200 Volts until the dye front was about 1 cm from the bottom of the gel. The gel was removed, wrapped in plastic and placed against photographic film for autoradiography.

Ligand Binding

Ten nM [³H] dexamethasone was added to 100 μg of GR transfected cos7 cell cytosol in the absence (for total binding) and presence of 500-fold excess unlabeled dexamethasone (for non-specific binding). RU486 or LF and/or PA were added as required. The samples for incubated overnight at 4° C. A sample was taken for scintillation counting. Bound ligand was separated from free by incubation with a 1% charcoal/0.1% dextran mix for 10 minutes followed by centrifugation. Again, a sample was taken for scintillation counting. Specific binding was determined as total binding—non-specific binding.

Phospho p38 ELISA

HTC and cos7 cells were lysed with M-PER (Pierce) on ice for 30 minutes with vortexing every 10 minutes. Phospho p38 MAPK [pTpY180/182] and total p38 MAPK were analyzed using phosphoELISA kits from Biosource International.

Cytotoxicity Assay

Cells were plated out in a 96 well plate and incubated at 37° C. until confluent. The drug of interest was then added and the cells incubated for the required length of time. Cytotoxicity was measured using the MTT based in vitro toxicity assay kit (Sigma) by addition of 10 μl of 5 μg/ml MTT (in PBS) three hours prior to the end of the experiment. After three hours the cells were lysed and the absorbance at 540 nm read.

### Example 2

### Lethal Toxin Repression of Dex-Induced Glucocorticoid Receptor Transactivation in Cos 7 Cells

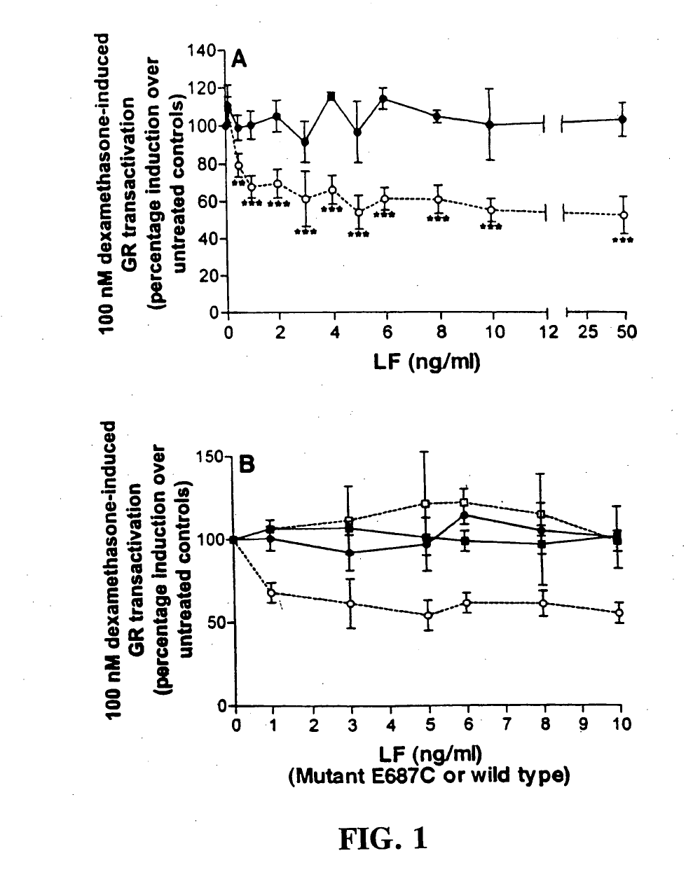

Cos7 cells, transiently transfected with the glucocorticoid receptor (SVGR) and a glucocorticoid response element (GRE)-luciferase reporter construct (GRE TK luc), were treated with 100 nM dexamethasone (Dex) and increasing concentrations of protective antigen (PA) or lethal factor (LF) in the presence or absence of saturating concentrations of the other lethal toxin (LeTx) component. FIG. 1 shows the relative transactivation of glucocorticoid receptor (GR) in response to 100 nM Dex in the presence of various combinations of increasing concentrations of LF, alone or together with PA. LF in the presence of 500 ng/ml PA (◯), but not alone (●), repressed GR (FIG. 1A) at concentrations as low as 0.5 ng/ml. Also, PA in the presence of 50 ng/ml LF, but not alone, repressed GR activity at concentrations as low as 5 ng/m. Maximal repression of GR by a combination of LF and PA at all concentrations was 50%. Even in the presence of LeTx, the system can be additionally and fully repressed by co-administration of the GR/PR antagonist RU 486 with LeTx, indicating that LeTx does not prevent the action of a pure anti-glucocorticoid at the ligand binding domain.

A single amino acid substitution mutant of LF, E687C, has been shown to be non-toxic in the LeTx sensitive macrophage cell line, RAW264.7 (K. R. Klimpel et al., Mol. Microbiol., 13:1093, 1994). This mutation has been shown to prevent proteolytic cleavage of a peptide while still allowing LF protein to bind zinc (K. R. Klimpel et al., Mol. Microbiol., 13:1093, 1994; S. E. Hammond et al., Infect. Immun., 66:23-74, 1998). In these transient transfection assays, in contrast to the repression induced by wild-type LF in the presence of PA (◯), the mutant LF (E687C) in the presence of 500 ng/ml PA (□) did not repress GR (FIG. 1B). This indicates that this particular amino acid is important for protein-protein interactions leading to GR repression or that the proteolytic activity of LF is required for GR repression.

### Example 3

### Comparison of the Effects of RU 486 and LeTX on the Dose Response Curve of Dex in GR-Transfected Cos7 Cells

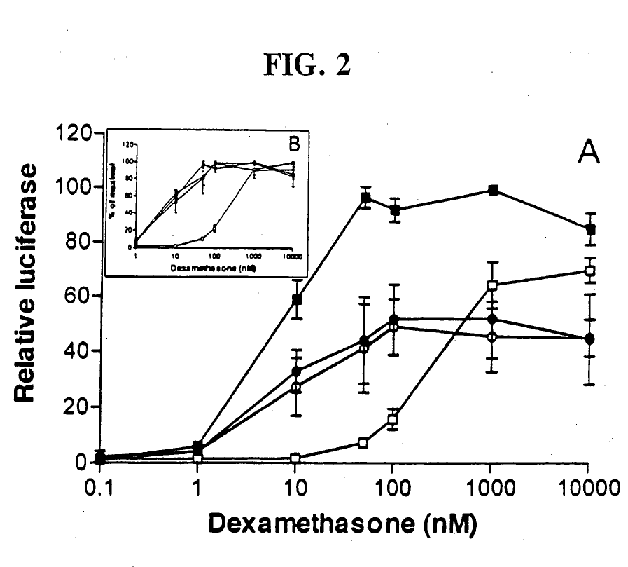

Full dose response curves of the normalized luciferase activity to Dex are shown in FIG. 2. FIG. 2A shows the effect of 500 ng/ml PA in combination with 10 ng/ml LF (●) or 50 ng/ml LF in combination with 5 ng/ml PA (◯) compared to Dex alone (▪) and to Dex plus the typical GR antagonist RU 486 (□). It can be seen that either combination of LF and PA caused approximately a 50% repression of GR at all effective concentrations of Dex. This pattern is indicative of a non-competitive repressor, in contrast to a competitive antagonist, such as RU 486, which can be fully competed out at higher concentrations of Dex. FIG. 2B (insert), with data presented as a percentage of the maximal activity in each case, shows that both combinations of LF and PA have no effect on the EC50 value, whereas the typical competitive antagonist RU 486 causes a right shift in the curve and an increase in the EC50. Thus, these pharmacological data indicate that LeTx represses GR activity in a non-competitive manner indicating that LeTx is not acting at the ligand-binding domain of the GR

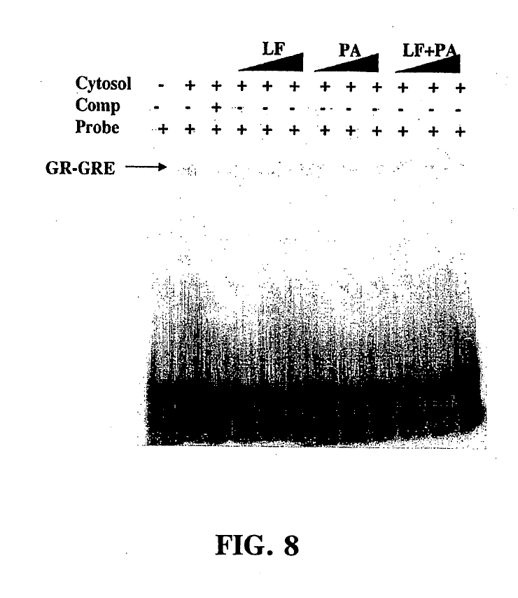

In addition, competitive ligand binding studies showed that in both whole cells and in cytosolic preparations neither LF nor PA, nor a combination of both, were able to compete with a saturating concentration of [³H] Dex for binding to GR These data, showing no effect of LeTx on [³H] Dex binding also demonstrate that LeTx has no effect on the number of functionally active GRs. Gel shift analysis of GR transfected cos7 cytosol to a radiolabelled oligonucleotide showed that LF, PA or a combination of both also had no effect on GR-DNA complex mobility (FIG. 8). Twenty-five μg of GR-transfected cos7 cytosol was incubated with a [32P] labeled GRE probe in the presence of 40 fold excess unlabeled probe as a competitor or with 5, 10 or 50 ng/ml LF, 10, 50 or 500 ng/ml PA, or with 5, 10 or 50 ng/ml LF in the presence of 500 ng/ml PA. The samples were run on a 40% Tris-borate-EDTA (TBE) acrylamide gel and visualized by autoradiography. The results indicate that LeTx does not interfere with GR-DNA binding, and indicating that it acts at a point down-stream of GR-DNA binding either by interfering with a co-factor or acting itself as a co-repressor. In addition, GR transfected cos7 cell cytosol forms a GR-GRE complex which is GR specific as it is competed out with excess unlabeled GRE probe. Increasing concentrations of LF and PA either alone or together have no effect on this complex indicating that LeTx does not prevent GR-DNA binding at least in an in vitro gel shift experiments.

### Example 4

### Comparison of the Effects of LeTX on the Mutant 407C and Wild Type GR

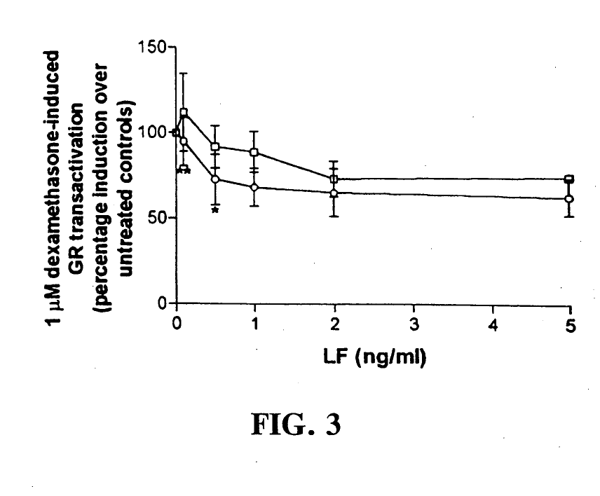

A mutant of GR that lacks the N-terminal transactivation domain (407C) but still contains the DNA binding domain (DBD) and ligand binding domain (LBD), exhibits lower transactivation activity than wild type GR but is also repressed by LeTx. At low concentrations of LeTx (0.1-0.5 ng/ml LF in the presence of 500 ng/ml PA) this 407C mutant (◯) shows a small but significantly greater repression of 1 μM dexamethasone-induced GR activity than wild type GR (□) (FIG. 3). This indicates that LeTx acts through the DBD and/or LBD domains of GR or through pathways that interact with these domains of the receptor.

### Example 5

### LeTX Repression of Dex-Induced Tyrosine Aminotransferase (TAT) in HTC Cells

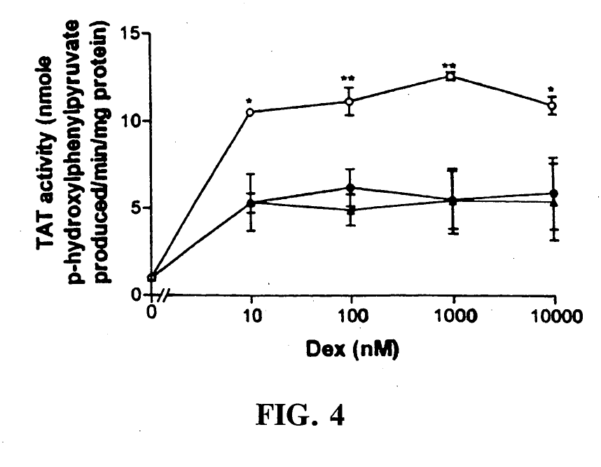

In order to determine whether the LeTx repression of GR gene activation observed in a transient transactivation system also occurs in a more natural system the effects of LeTx on dexamethasone induction of the GR regulated enzyme tyrosine aminotransferase (TAT) was investigated in a rat hepatoma cell line (HTC-cells) (FIG. 4). HTC cells were treated for 18 hours with increasing concentrations of Dex either alone (◯), or together with 2 ng/ml LF in the presence of 500 ng/ml PA (●) or 10 ng/ml LF in the presence of 500 ng/ml PA (▴) and TAT enzyme activity was assayed. TAT activity was induced approximately 10-fold by Dex concentrations as low as 10 nM. Co-treatment with either 2 ng/ml or 10 ng/ml LF in the presence of 500 ng/ml PA reduced Dex induction of TAT activity by 50%, in agreement with the transient transfection assays.

### Example 6

### Comparison of the Effects of LeTX and PD98059 on the Response of a Dex-Induced GRE Luciferase and a Constitutive Luciferase (pGL3) Control

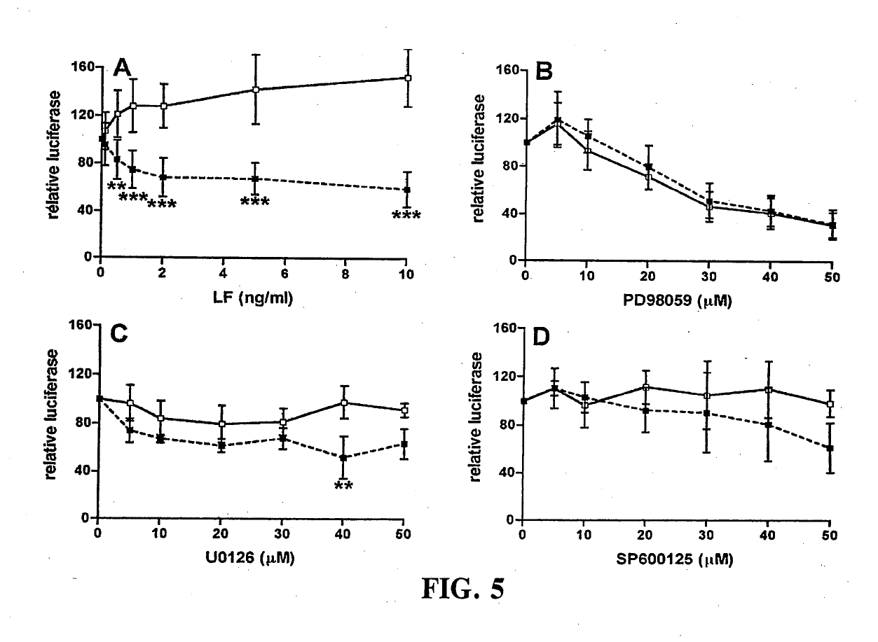

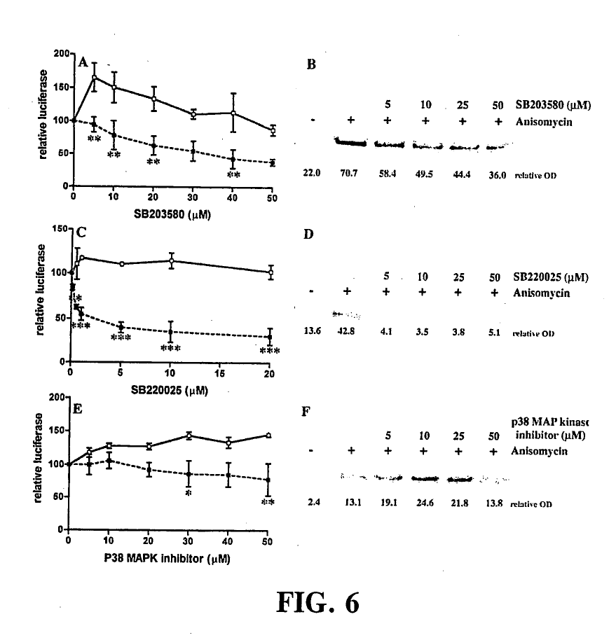

Known substrates for the proteolytic action of LF include some members of the MAP kinase family (MAPKKs). The cleavage of these proteins results in a blockage of the MAP kinase pathway. Cell lines exhibiting differential sensitivity to LeTx toxicity exhibit a similar sensitivity profile to the MEK1 inhibitor PD98059. When the effect of this MEK1 inhibitor on GR transactivation was compared to the effects of LF, PD98059 decreased luciferase activity of both the GRE-luciferase (▪) and the constitutive luciferase vector (pGL3 control) (□) to the same extent (FIG. 5), whereas LF in the presence of PA had no effect on the pGL3 control (FIG. 6).

In FIG. 5, Cos7 cells were transfected with SVGR and (GRE)₂-TK luc (▪) or with SVGR and the constitutive luciferase vector, pGL3 control (Promega) (□) and treated with 100 nM dexamethasone, and increasing concentrations LF with 500 ng/ml PA (FIG. 5), or increasing concentrations of the MEK1 inhibitors, PD98059 (FIG. 5B), and U0126 (FIG. 5C) or the JNK inhibitor, SP600126 (FIG. 5D). Means and standard deviations are shown and data was analyzed using a two-way ANOVA followed by a Scheffe post hoc test.

In FIG. 6, Cos7 cells were transfected with SVGR and (GRE)₂-TK luc (▪) or with SVGR and the constitutive luciferase vector, pGL3 control (□) and treated 100 nM dexamethasone, and increasing concentrations of the p38 MAP kinase inhibitors, SB203580 (FIG. 6A), SB220025 (FIG. 6C) and p38 MAP kinase inhibitor (FIG. 6E). Means and standard deviations are shown and data was analyzed using a two-way ANOVA followed by a Scheffe post hoc test. Cos7 cells were pre-treated for 30 min with various concentrations of SB203580 (FIG. 6B), SB220025 (FIG. 6D) or p38 MAP kinase inhibitor (FIG. 6F) and then further incubated with 10 μg/ml anisomycin for 30 min. Proteins were then subjected to SDS-PAGE and Western blotting using an anti-phospho-p38 antibody.

These results show that PD98059 has a non-specific suppressive effect on luciferase, occurring through unknown mechanisms, in this transient transfection system. Furthermore, these data show that the PD98059 inhibitor does not induce any GRE-specific changes in luciferase and therefore does not affect GR transactivation. These results also indicate that the mechanism of the effect of LF and PD98059 on GR transactivation activity is different and that the LF repression of GR probably does not occur through inhibition of the MEK1 pathway. SB203580, an inhibitor of the p38 pathway also has no effect on GR-mediated transactivation in a GRE-luciferase system.

Although LF also functions as an inhibitor of the MEK4/7 and MEK3/6 pathways, this result is consistent with previous literature showing that while activation of the MAPK pathway can repress GR, either through activation of ERK and JNK (M. D. Krstic et al., Mol. Biol. Cell., 17:3947, 1997; G. N. Lopez et al., J. Biol. Chem., 276:22-177, 2001; I. Rogatsky et al., Proc. Natl. Acad. Sci. U.S.A., 95:20-50, 1998), or activation of c-Fos and c-Jun (F. C. Lucibello et al., EMBO J., 9:2827, 1990; R. Schule et al., Cell., 62:12-17, 1990; P. Herrlich, Oncogene, 20:24-65, 2001; M. Karin et al., J. Endocrinol., 169:447, 2001), there is no evidence to date that a blockage of the MAPK pathway can result in GR repression.

The theory behind these experiments is that if LeTx is mediating its effect on GR through its ability to cleave and inactivate members of the MAPK family then inhibitors of these pathways should have a similar effect in out GR transfection system. FIG. 5 shows that inhibitors of the MEK/ERK (PD98059 and U0126) or JNK (SP600125) pathways have no GRE specific effect. However, FIG. 6 shows that inhibitors of the p38 pathway do have a repressive effect on the dexamethasone induced GR transactivation in this system and that this repression appears to be correlated with the inhibitors efficacy as a p38 inhibitor in these cells. This indicates that the p38 pathway is involved.

### Example 7

### Effects of LeTX on Hormone-Induced Activity of Other Nuclear Hormone Receptors

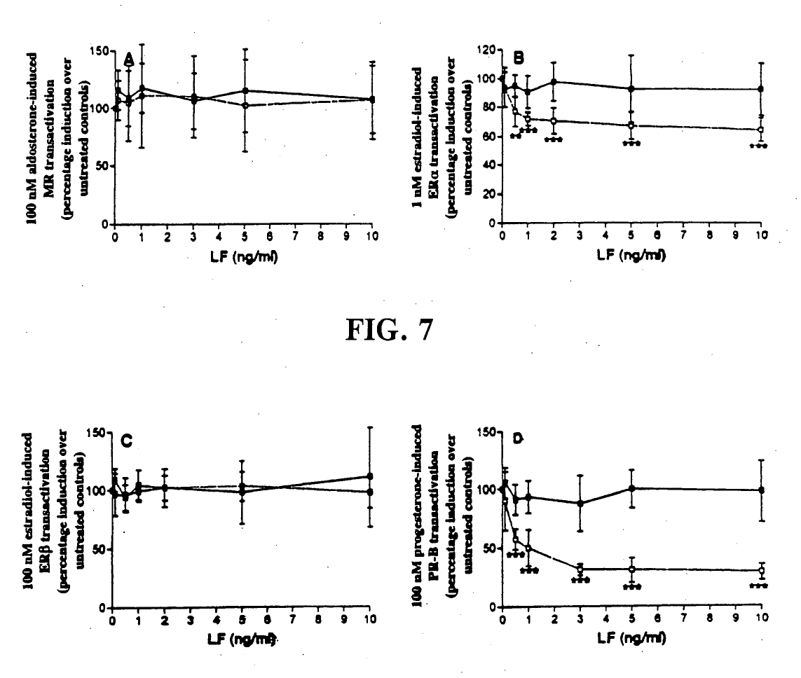

In order to determine whether the GR repression by LeTx is specific for GR or affects other nuclear hormone receptors, transient transfection experiments were performed using the receptors for estrogen (ER)α, ERβ, mineralocorticoid (MR) and progesterone B (PR-B) and their respective reporter plasmids. In contrast to its 50% repression of GR, LeTx had no effect on MR (FIG. 7A). LeTx repressed ERα by approximately 40% (FIG. 7B) but had no effect of ERβ (FIG. 7C). Finally, LeTx repressed PR-B by 70% (FIG. 7D). Thus, LeTx represses nuclear hormone receptor transactivation in a partially specific manner, affecting some but not all members of this hormone receptor family.

### Example 8

### Evaluation of Nuclear Hormone Receptor Cofactors for their Potential Roles in LeTx-Mediated Nuclear Hormone Receptor Repression

SRC1, TIF2 and CBP are co-factors that are known to interact directly with the ligand binding domain (LBD) of nuclear hormone receptors such as GR. Co-transfection of SRC1, TIF2 or CBP was undertaken according to known methods to achieve expression of these cofactors in a suitable host cell, and the rescue effect of this expression on LeTx-mediated GR repression was evaluated. Co-transfection of SRC1, TIF2 or CBP to determine whether had no-effect on LeTx repression of GR. An effect of TIF2 alone was observed, in which this co-factor significantly enhanced the GR transactivation. However, LeTx repressed GR transactivation 40-50% in the presence or absence of TIF2. These findings indicate that LeTx does not function directly through or prevent the action of these co-factors. Similar to their lack of effect on GR transactivation, co-transfection of SRC1, TIF2 or CBP had no effect on LeTx repression of PR-B. TIF2 similarly enhanced the progesterone-induced PR-B transactivation in the absence of LeTx, and in the presence of LeTx the toxin's 70-80% repression was maintained even with addition of 100 ng TIF2. This indicates that these co-factors are not directly involved in LeTx repression of PR-B. Additional proteins identified as cofactors (including co-activator and co-repressor proteins), as described herein above, will therefore be evaluated using similar cotransfection/rescue assays to determine those cofactors that are directly or indirectly involved in LeTx-mediated repression of nuclear hormone receptor function and that will therefore provide additional screening and diagnostic tools and therapeutic compositions and methods in accordance with the instant disclosure.

Taken together, the foregoing examples demonstrate that LeTx represses transactivation of both a transiently transfected and an endogenous GR-regulated gene. This repression is non-competitive and does not affect ligand binding or DNA binding, indicating that LeTx likely exerts its effects through a cofactor(s) involved in the interaction between GR DBD/LBD and the basal transcription machinery.

LeTx exhibits a maximum of 50% repression of GR and 70% repression of PR-B. Such partial repression is indicative of the target of LeTx being down-stream of GR-DNA binding in the interaction of GR with the basal transcription machinery. As there are multiple proteins involved in this interaction, if one component is removed and/or repressed, then remaining, intact co-factors could still allow some but not full activity of the receptor.

The ability of LeTx to repress the 407C mutant GR, which lacks the N-terminal transactivation domain, indicates that proteins that interact with the DBD and/or LBD of GR are involved directly or indirecely in this repression. The small but significantly greater repression at low concentrations of LeTx indicates that the N-terminal transactivation domain of wild-type GR may be slightly protective of this repression.

Contrary to previous models proposed in the literature, the MEK1 pathway is probably not involved in LeTx activity. The MEK1 inhibitor PD98059 did not alter GR repression in a transient transfection assay. The specificity of repression of some but not all members of the nuclear hormone receptor family tested also supports the notion that LeTx is working through a co-factor rather than through a direct interaction with the GR receptor.

In light of the foregoing description, LeTx repression of nuclear hormone receptors in vivo in the course of anthrax infection likely contributes to some of the adverse symptoms of anthrax. Since the glucocorticoid receptor is essential for survival and also for modulation of immune responses to infectious agents, inhibition of glucocorticoid receptor activity during infection is proposed to render the host more susceptible to the lethal or toxic effects of anthrax bacteria. Simultaneous loss of activity of other nuclear hormone receptors, particularly PR, would potentially amplify these immune enhancing effects. Indeed, this scenario is consistent with the well-described increased mortality from septic shock in rodents that have been adrenalectomized or treated with the GR/PR receptor antagonist RU 486, and simultaneously exposed to infectious agents or pro-inflammatory bacterial products (C. K. I. Edwards et al., Proc. Natl. Acad. Sci. U.S.A., 88:2274, 1991; E. M. Sternberg et al., Proc. Natl. Acad. Sci. U.S.A., 86:2374, 1989; M. C. Ruzek et al., J. Immunol., 162:3527, 1999; I. A. M. MacPhee et al., J. Exp. Med., 169:431, 1989). The GR repression by LF could also contribute to the long-term inflammatory and fatigue sequelae now being reported in relation to anthrax exposure (J. A. Jernigan et al., Emerg. Infect. Dis., 7:933, 2001), since blunted glucocorticoid responses have been associated with many inflammatory diseases and fatigue states (G. Neeck et al., Rheum. Dis. Clin. North Am., 26; 989, 2000). Application of the compositions and methods provided herein to further map nuclear hormone receptor co-factor interactions as a mechanism of in vivo action of anthrax LF will thus yield important new tools for treatment and prevention of the adverse effects of this toxin and other bacterial products having similar activities.

In accordance with the foregoing results and additional teachings herein, additional, confirming studies will be undertaken to identify more specific aspects of the subject technology, in particular more specific aspects of the molecular mechanism(s) of LF/PA effect on GR and other nuclear hormone receptors. Certain molecular studies will focus on elucidating the precise molecular mechanism(s) by which LeTx interacts with and represses GR and other nuclear hormone receptors; determining whether LeTx interacts with a GR and other nuclear hormone receptor co-factors or acts as a co-repressor itself; and determining whether LeTx can affect GR and other nuclear hormone receptor gene repression as well as gene activation.

### Example 9

### Effect of LeTx on GR Gene Repression

Since the mechanism of GR repression and activation of genes differs, LeTx also may affect GR-mediated gene repression in addition to the repression described in the foregoing examples. In order to elucidate these further aspects of the disclosure, transient transfection experiments comparable to those presented above are performed using cells transfected with known vectors encoding NFκB or AP-1 and their respective reporter constructs, together with increasing concentrations of GR. Cells are then treated with appropriate ligand for NκB and AP-1 and Dex together with increasing concentrations of LeTx. GR gene repression is measured in the luciferase reporter system as described for the GRE-reporter.

### Example 10

### Identification of Co-Factors Involved in LeTx Effect

In order to identify which co-factors (co-activators/co-repressors) are affected by LeTx, and to determine if the GR repression by LeTx can be overcome by supplementation with such co-factors, key members of each of the major families of cofactors (SRC-1, TIF2, pCIP (AIB1), CBP and pCAF) are co-transfected in increasing amounts into the GR/GRE transiently transfected Cos 7 cells and GR activation is measured in the GRE-luciferase transactivation assay in the presence and absence of a range of doses of LF and PA alone or together. Expression plasmids for a large panel of cofactors are readily obtained from academic, institutional and commercial sources in the art, and these expression vectors can be readily utilized in transient transfection and related assays available in the art. A large number of cofactors can be evaluated by these assays, including well-known high throughput assays, for use within the methods and compositions of the disclosure. Among the subject cofactors for use within these screening aspects of the disclosure are those listed in the exemplary listing provided in Table 2 above.

Thus, a method is provided for identifying a nuclear hormone receptor cofactor that is an agonist or antagonist of a selected nuclear hormone recepetor. The method includes the steps of:
(1) providing a viable test cell that expresses the cofactor and the nuclear hormone receptor, and a substrate/reporter construct for the nuclear hormone receptor, wherein expression of the substrate reporter construct is detectable and provides a measurement of nuclear hormone receptor pathway activity;
(2) providing a viable control cell that expresses the nuclear hormone receptor and the substrate/reporter construct for the receptor but has reduced or no expression of the cofactor in comparison to cofactor expression in the test cells;
(3) contacting the test and control cells with a bacterial product that modulates the nuclear hormone receptor pathway;
(4) detecting and comparing nuclear hormone receptor pathway activity between the test and control cells to determine whether the cofactor enhances or impairs modulation of the receptor pathway activity by the bacterial product.

### Example 11

### Dissection of Region of GR Involved in LeTx Repression

The region of GR required for LF/PA repression is defined according to known methods using mutant and chimeric forms of the GR. The mutant and chimeric constructs are transiently transfected into Cos 7 cells in a transient transfection assay as described above. Activation of the GR is assessed in the presence and absence of a range of doses of LF and PA alone or together. In exemplary embodiments, several available mutants lacking specific regions of GR, and known chimeras of PR/GR and MR/GR are used. These include, for example: 407C—lacks a transactivation domain, contains DBD and LBD of GR (D. Szapary et al., J. Biol. Chem., 271 :30576-82, 1996), GR/PR—transactivation domain and DBD of GR and hinge and LBD of PR; PR/GR—transactivation domain and DBD or PR and hinge and LBD of GR (L. N. Song et al., J. Biol. Chem., 276:24806-16, 2001); MR/GR chimeras containing the N-terminal domain of GR and the DBD and LBD or MR and vice versa.

### Example 12

### Interactions Between LF/PA and Components of the GR-GRE Complex

Gel shift analyses have not shown an effect of LF/PA on the GR-GRE complex. This in vitro system indicates that LeTx does not interact directly with the GR-GRE complex, as it does not further shift this band. However, there are many proteins in the GR transactivation complex, downstream of GR-GRE, with which LF/PA may interact to affect GR responses without any direct interaction with GR-GRE. Therefore, in order to elucidate direct interactions between LF/PA and identifiable components of the GR-GRE complex or co-factors, co-immunoprecipitation studies are performed using available polyclonal and monoclonal antibodies to LF. In these assays, OR is obtained from cell lysates and dexamethasone and LF+PA is added to the mixture. A parallel set of experiments is also performed using whole cells. Known proteins (LF, GR, MAPK) are identified in gel shift assays, for example, by Western blotting. Unknown proteins in the complex are identified according to well-known methods (for example, mass spectrometry). If co-immunoprecipitation is insensitive, GST (glutathione-S-transferase) LF pull-downs are performed to identify whether any direct interactions with any components of the GR complex occur.

### Example 13

### Response of Endogenous GR-Regulated Genes to LeTx

The effects of LeTx on expression of endogenous genes known to be induced or repressed by glucocorticoids are further assessed in intact cell lines and primary cell cultures. Genes known to be repressed by GR include, for example, IL-6, TNFα, collagenase and COX-2, via the NFκB and AP-1 pathways. Genes known to be activated by GR include metallothionein IIa, tyrosine amino transferase (TAT), phosphoenolpyruvate carboxykinase (PEPCK) and glutamine synthase (GS). In these assays, mRNA, protein expression or enzyme activity of dexamethasone regulated genes is measured according to conventional methods in cell lines that contain endogenous GR but in which LeTx is non-toxic.

### Example 14

### Additional Nuclear Hormone Receptors and Domains

To identify other nuclear hormone receptors modulated by LeTx and other bacterial products, transient transfection systems as outlined above are employed. For example, the effect of LeTx and other bacterial products (for example, as identified in Table 1 above) on a panel of nuclear hormone receptors, including androgen receptor (AR), mineralocorticoid receptor (MR), progestin receptor (PR), estrogen receptor (ER), thyroid hormone receptor (TR), vitamin D receptor (VDR), retinoid receptor (RAR or RXR), peroxisome receptor (XPAR or PPAR), icosanoid receptor (IRs), and orphan receptors, for example steroid receptor and thyroid receptor.

In one embodiment, a method is disclosed that is a method of identifying a domain or amino acid sequence motif of a nuclear hormone receptor involved in modulation of activity of the nuclear hormone receptor by a bacterial product. The method includes the steps of providing a viable test cell that expresses a mutant, chimeric, or truncated form of the nuclear hormone receptor and a substrate/reporter construct for the nuclear hormone receptor, wherein expression of the substrate reporter construct is detectable and provides a measurement of nuclear hormone receptor pathway activity; providing a viable control cell that expresses a full-length or functionally wild type nuclear hormone receptor and the substrate/reporter construct for the receptor; contacting the test and control cells with a bacterial product that modulates the wild type nuclear hormone receptor pathway; and detecting and comparing nuclear hormone receptor pathway activity between the test and control cells to determine whether the cofactor enhances or impairs modulation of the receptor pathway activity in the cells expressing the mutant, chimeric, or truncated form of the nuclear hormone receptor. Thus, a determination is made whether structural elements present in the mutant, chimeric, or truncated form of the receptor are involved in modulation of activity of the nuclear hormone receptor by a bacterial product by the bacterial product. In some embodiments of the method, the bacterial product is a bacterial toxin, for example, anthrax lethal factor (LF) or lethal toxin (LeTx). In particular examples, the nuclear hormone receptor is selected from glucocorticoid receptor (GR), progestin receptor (PR), and estrogen receptor-α (ER-α).

### Example 15

### In Vivo and Clinical Relevance of Nuclear Hormone Receptor Repression by LeTx

To further elucidate the clinical significance of LeTx-GR/nuclear hormone receptor interactions as they relate to inflammation, autoimmunity, toxicity and lethality associate with anthrax and other bacterial diseases, and their cognate vaccines, the following studies are performed. Attendant goals in this context include:

i. To determine in vitro whether macrophages from rat strains differentially susceptible to anthrax LeTx, or cell lines that differ in susceptibility and resistance to anthrax LeTx show differences in GR number, affinity, function or cytotoxicity to GR antagonists.

ii. To elucidate how nuclear hormone receptor repression mediated by bacterial products alters inflammation, autoimmunity, toxicity and lethality associate with anthrax and other bacterial diseases, and their cognate vaccines.

iii. To identify other bacterial toxins that act as GR and/or other nuclear hormone receptor repressors.

### Example 16

### In Vitro Cell Culture Studies of Macrophage GR Number and Function in LeTx Resistant and Susceptible Macrophages

Several macrophage cell lines exist that are relatively sensitive (J744.1 and RAW264.7) or resistant (IC-21 and MT-2) to cytotoxicity after exposure to LeTx. Since MAPKK degradation by LeTx does not differ in these sensitive and resistant cells lines, an additional factor(s) must contribute to their differential sensitivity. GR number, binding characteristics and function in these cell lines are evaluated in order to further define the contribution of endogenous differences in GR function in this differential sensitivity. While a lack of difference in GR function does not rule out the involvement of GR or its co-factors in LeTx differential toxicity, small differences in GR function, compounded by LeTx GR repression, may account for such differences.

GR number and function of peritoneal macrophages from F344/N and LEW/N rats are evaluated in parallel, since F344/N rats are more susceptible to the lethal effects of in vivo administered LeTx than are LEW/N rats. GR number and affinity are readily measured, for example, using radiolabeled ³H-Dex in ligand binding assays. Function is assessed, for example, by evaluating endogenous GR activated or GR repressed genes, as described above.

In addition, it will be determined whether the GR/PR ligand-binding antagonist RU486 is differentially toxic to, or reverses the sensitivity and resistance of, macrophages to LeTx. In these assays, RU486 is added alone or together with LeTx in varying doses to sensitive and resistant cell lines and cytotoxicity are measured, for rexample, in a standard MTT cytotoxicity assay (Sigma, Mo.)

Finally, expression of other factors identified through the above-described molecular studies are evaluated and quantified, and further assays developed to reconstitute missing/defective factor(s) to determine whether nuclear hormone receptor repression by LeTx and other bacterial products can be overcome by such replacement.

### Example 17

### Int vivo GC Antagonism by Bacterial Products and HPA Axis

(a) Differential Pre-Morbid HPA Axis Responsiveness and Differential Strain Susceptibility to Anthrax Lethality:

To evaluate clinical aspects of the disclosure, for example how pre-morbid HPA axis responsiveness is associated with differential strain susceptibility to anthrax, clinical effects on blood pressure, heart rate and temperature, chronic inflammation, autoimmune effects, and lethality are assessed according to various protocols. For example, hyper-HPA axis responsive F344/N rats and hypo-HPA axis responsive LEW/N rats are employed as test subjects. If differential responses are found, both strains of rats are treated with Dexamethasone (Dex) to determine whether Dex replacement overcomes or prevents the symptoms. However, as in vitro studies indicate that LeTx is an irreversible GC repressor, it is unlikely that Dex would prevent or overcome the toxic effects of LeTx. Testing of agents to counter the effects of LeTx is informed by the outcome of in vitro molecular mechanism studies.

(b) LeTx Acute Effects In Vivo on LPS-Induced Inflammatory Responses, Septic Shock and HPA Axis Responses:

To further evaluate how LeTx acts as a GR antagonist in vivo, F344/N rats are treated with bacterial lipopolysaccharide (LPS) as a stimulus to the HPA axis at the same time as a range of sub-lethal doses of LeTx are administered intra-peritoneally as a GR antagonist. Studies are performed as previously described for SCW and RU486 experiments (E. M. Sternberg et al., Proc. Natl. Acad. Sci. U.S.A., 86:2374-8, 1989). Plasma levels of corticosterone, plasma cytokines that are usually released during septic shock (TNF-α, IL-6 and IL-1), as well as blood pressure, heart rate and temperature are monitored at different time points prior to and after treatment over a one hour period. Mortality in different groups is recorded. In these assays, LeTx antagonism of GR and PR is predicted that it might have a similar effect as other GR/PR antagonists for example, RU 486, leading to rapid death from septic shock by blocking the anti-inflammatory effects of glucocorticoids. Plasma corticosterone and ACTH responses are predicted to increase, depending on the degree to which LeTx blocks glucocorticoid negative feedback of the HPA axis and peripheral cytokine production. LeTx blockade of the effects of GC in suppressing the HPA axis, plasma Cort and ACTH are expected to increase, resulting in a situation of high plasma Cort and relative peripheral GC resistance.

(c) Ex Vivo Measurement of GR-Mediated Gene Induction and Gene Repression.

Glucocorticoid (GC) sensitivity is assessed using a whole blood dexamethasone induction and LPS-stimulation/GC-suppression assay previously described (R. H. DeRijk et al., Journal of Clinical Endocrinology and Metabolism, 81:228-35, 1996). Whole blood is also be stimulated ex vivo with LPS, and cytokine production in supernatants is measured in the presence and absence of varying doses of dexamethasone (Dex) +/−LF +/−PA. RU486 in varying doses is used as a positive control. RU486 blocks the glucocorticoid receptor, thereby preventing the Dex suppression of LPS-induced cytokines. Concentrations of cytokines in supernatants are measured by ELISA and/or by immunoaffinity capillary electrophoresis (T. M. Phillips et al., Electrophoresis, 19:2991-6, 1998).

(d) In Vivo Co-Factor Effects on LeTx Toxicity:

In conjunction with the above-described molecular studies, and in order to further assess how co-factors identified as targets of LeTx in vitro operate in LeTx's toxicity in vivo, knock-out mice not expressing the identified co-factor(s) and transgenic mice over-expressing the identified co-factor(s) are studied. The animals are treated with a range of doses of LeTx and HPA axis responses, cytokine and blood pressure, temperature, and any other sickness responses are compared to wild-type. In addition, the effects of LeTx are tested in GR dimerization mutant (GR^{dim/dim}) mice, in which GR gene repression occurs, but GR gene activation does not occur. A range of concentrations of LeTx or vehicle control is administered to knock-out and wild-type controls and HPA axis responses, cytokine and blood pressure, temperature and any other sickness responses are compared to wild-type. GR^{dim/dim} dim mice are obtained from Dr. Jan-Ake Gustafsson, Karolinska Institute, Stockholm, Sweden.

(e) Chronic In Vivo Effects of LeTx on Inflammatory/Autoimmune Disease in Animal Models:

The manner and mechanisms by which LeTx operates in widely accepted models of inflammatory and autoimmune diseases is assessed. LEW/N and F344/N rats are injected subcutaneously with complete Freund's adjuvant as previously described (Webster et al., J. Rheumatol. 29:1252-61, 2002) and LeTx or vehicle control are simultaneously administered intraperitoneally in sub-lethal doses selected from pilot studies using LeTx alone. Rats are scored daily for four weeks for arthritis severity (arthritis index) and body weight, as previously described. At the end of this period a full autopsy is performed, and tissues, including synovial tissue, are analyzed for evidence of inflammation.

In addition, another model for use within these aspects of the disclosure is the model of relapsing murine experimental allergic encephalomyelitis induced by myelin basic protein (the EAE model). This widely accepted animal model for evaluating treatments for multiple sclerosis is described, for example, in Fritz et al., J. Immunol. 130: 1024-6, 1983. A related model has been described using rat subjects by MacPhee et al., J. Exp. Med. 169:43145, 1989. In this model, EAE is induced in Lewis rats and causes paralysis. Endogenous glucocorticoids ameliorate the effects of EAE. Adrenalecomized rats were implanted with a coritcosterone pellet. If it mimicked the basal GC levels, then the animals died. If it mimicked the GC levels during the EAE disease, then the animals survived and the level of disease was comparable to non-adrenalectomized animals. If the GC levels were higher then disease remission was achieved. These models are therefore useful in the context of assays to evaluate the clinical significance and mechanisms of bacterial product suppression of GR and associated impacts on autoimmune diseases.

### Example 18

PA and/or LF Do Not Prevent [³H] dexamethasone Binding to GR Transfected Cos7 Cell Cytosol Preparations

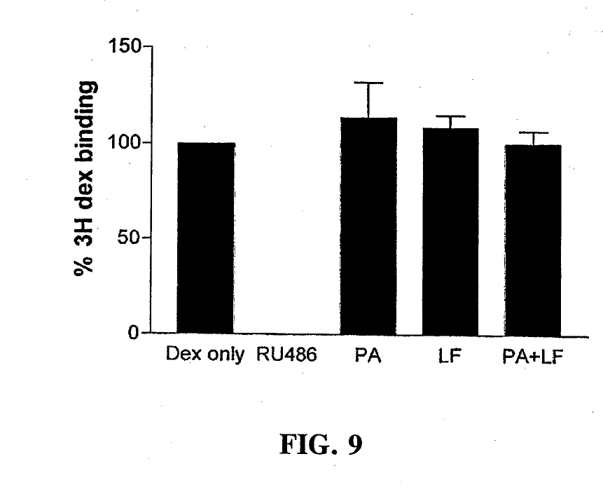

This example demonstrates that PA and/or LF do not prevent [³H] dexamethasone binding to GR transfected cos7 cell cytosol preparations. One hundred μg GR transfected cos7 cytosol was incubated overnight with 10 nM [³H] dexamethasone in the presence or absence of 500 fold excess unlabeled dexamethasone and in the presence of 1 μM RU486, 500 ng/ml PA, 50 ng/ml LF or 500 ng/ml PA+50 ng/ml LF. Bound was separated from free and specific binding calculated. The percent specific binding in comparison to dexamethasone alone is shown (FIG. 9). These results show that RU486 can compete with a saturating concentration of 3H dexamethasone where as PA, LF or LF+PA cannot. Therefore LeTx does not function as a normal GR antagonist such as RU486 in that it does not compete with dexamethasone for ligand binding. Also, if there were a decrease in the number of glucocorticoid receptors one would expect the amount of saturating ³H dexamethasone binding to decrease. Therefore, LeTx does not effect the number of glucocorticoid receptors. This result has been confirmed with Western blotting.

### Example 19

### RU486 Can Fully Repress Dexamethasone-Induced GR Transactivation and Progesterone-Induced PR-B Transactivation in Cos7 Cells Even in the Presence of LeTx

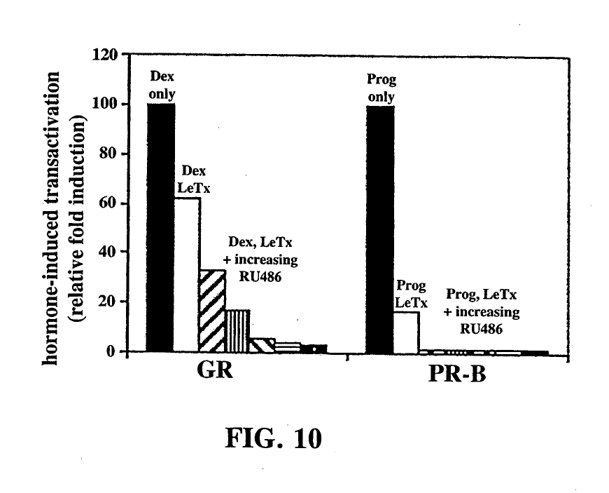

This example demonstrates that RU486 can fully repress dexamethasone-induced GR transactivation and progesterone-induced PR-B transactivation in cos7 cells even in the presence of LeTx. Cos7 cells were transfected with SVGR and (GRE)₂-TK luc or PR-B and pLTR luc and then treated with 100 nM dexamethasone or progesterone in the presence of 2 ng/ml LF+500 ng/ml PA and increasing concentrations of RU486 (maximum 1 μM). Relative luciferase values were measured (FIG. 10). These results show that addition of RU486 in combination with LeTx allows full repression of both GR and PR-B. This indicates that transcription can be fully repressed in the presence of LeTx.

### Example 20

### Over Expression of TIF2 Does Not Overcome LeTx Repression of Dexamethasone-Induced GR Transactivation

This example demonstrates that over expression of TIF2 does not overcome LeTx repression of dexamethasone-induced GR transactivation. Cos7 cells were transfected with SVGR and (GRE)₂-TK luc and increasing amounts of TIF2 expression plasmid (maximum 100 ng) and then treated with 100 nM dexamethasone in the presence of 2 ng/ml LF+500 ng/ml PA or 10 ng/ml LF+500 ng/ml PA. Relative luciferase values were measured. Relative fold induction and percent repression by LeTx are shown. If LeTx is removing one of the many cofactors involved in the interaction between the GR/PR-B and the transcriptional machinery, then over-expression of this factor may overcome the repression. CBP, SRC-1 and TIF2 are the major cofactors that are known to interact directly with these nuclear hormone receptors. Over-expression of these (CBP, TIF2 and SRC-1) was unable to overcome the repression of GR or PR-B by LeTx.

### Example 21

### LeTx Repression of Dexamethasone Induced Tyrosine Aminotransferase (TAT) in Mouse Livers

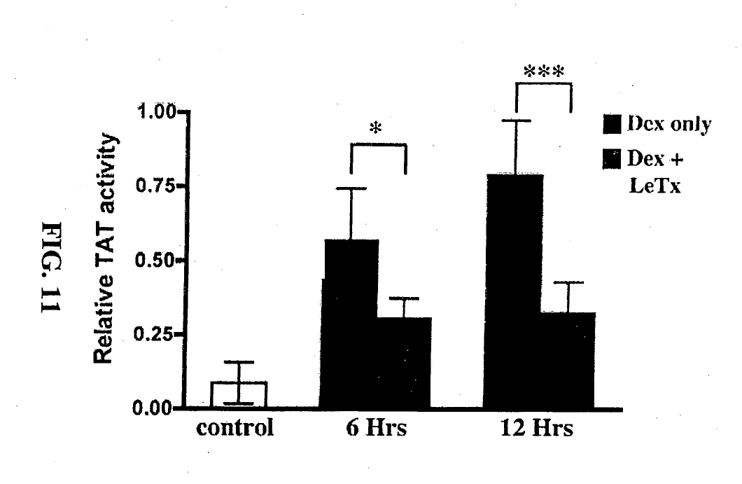

This example demonstrates that LeTx repression of dexamethasone induced tyrosine aminotransferase (TAT) in mouse livers. BALB/cJ mice were injected with LeTx and 30 minutes later with Dex. After six and twelve hours liver TAT activity was assayed (FIG. 11). Means and standard deviations of six to ten animals are shown and a two-way ANOVA followed by a Scheffe post hoc test was performed. These results show that LeTx is able to also repress dexamethasone induction of tyrosine aminotransferase (TAT) activity in mouse livers.

### Example 22

### MAPK Inhibitors Repress Dexamethasone-Induced TAT Activity in HTC Cells

This example demonstrates that MAPK inhibitors repress dexamethasone-induced TAT activity in HTC cells. HTC cells were treated with dexamethasone either alone (dex) or together with 2 ng/ml LF+500 ng/ml PA, 50 μM PD98059, 50 μM U0126, 50 μM SP600125, 50 μM SB203580 or 20 μM SB220025 for 18 hr and TAT activity assayed. MAPK inhibitors were tested to determine whether they were able to repress dexamethasone induction of TAT in HTC cells. They did have an effect in on TAT induction, although this system cannot distinguish between GR specific and non-specific effects as the cos7 cells. However, these results indicate that the p³⁸ repression of GR is not an artifact due to the cos7 cells, but also occurs in these HTC cells.

### Example 23

### LeTx Inhibits Endogenous Phospho P38 in GR Transfected Cos7 Cells and HTC Cells

This example demonstrates that LeTx inhibits endogenous phospho P38 in GR transfected cos7 cells and HTC cells. Phospho P38 (FIGS. 14 A and 14 B) and total P38 (FIGS. 14C and 14D) were measured by phosphoELISA in samples of GR transfected cos7 cells (FIGS. 14A and 14C) or HTC (FIG. 14B or 14D) treated with 100 nM Dexamethasone and increasing concentrations of LF in the presence of 500 ng/ml PA.

This experiment was designed to determine the relative content of p38 and phospho p38 in cos7 and HTC cells treated with dexamethasone and LeTx. These results show that LeTx does indeed repress p38 (as shown by a decrease in phospho p38) in the LeTx experiments. Thus, together with the data in Example 22, this example shows that during out LeTx repression of GR experiments in both cos7 and HTC cells the LeTx also represses P38. In addition, inhibition of p38 correlates with repression of GR.

### Example 24

### J774.1 and Raw264.7 Macrophage Cell Lines are Sensitive to LeTx Whereas IC-21 and MT2 Macrophage Cell Lines are Relatively Resistant

This example demonstrates that J774.1 and Raw264.7 macrophage cell lines are sensitive to LeTx whereas IC-21 and MT2 macrophage cell lines are relatively resistant. J774.1, Raw264.7, IC-21 and MT2 cells were grown in DMEM and exposed to increasing concentrations of LF in the presence of 500 ng/ml PA for 24 hours. MTT assay was performed at the end of the 24 hours and the percent cell survival is shown as the percentage cells surviving compared to cells that have not been exposed to LeTx. Thus, there exist macrophage LeTx sensitive and resistant cell lines.

### Example 25

### Pretreatment of Dexamethasone or RU486 Does Not Prevent LeTx Toxicity in J744.1 or Raw264.7 Cell Lines

This example demonstrates that pretreatment of dexamethasone or RU486 does not prevent LeTx toxicity in J744.1 or Raw264.7 cell lines. J774.1 and Raw264.7 cells were grown in DMEM and exposed to increasing concentrations of LF or LFm (E687C) in the presence of 500 ng/ml PA for 24 hours. In some cases the cells were pre-treated with 100 nM dexamethasone or 0.2 μM or 1 μM RU486 for 2 hours prior to LeTx treatment. MTT assay was performed at the end of the 24 hours and the percent cell survival is shown as the percentage cells surviving compared to cells that have not been exposed to LeTx. Thus, co treatment with with dexamethasone or RU486 has no effect on the LeTx cytotoxicity of the sensitive cell lines.

### Example 26

### Rolipram Does Not Prevent LeTx Repression of Dexamethasone-Induced TAT Activity in HTC Cells

This example demonstrates that rolipram does not prevent LeTx repression of dexamethasone-induced TAT activity in HTC cells. HTC cells were treated with 1 or 10 μM dexamethasone and/or 10 μM rolipram either alone (treatment) or together with 2 ng/ml LF+500 ng/ml PA or 10 ng/ml LF+500 ng/ml PA for 18 hours and TAT activity assayed. A drug that activates GR and circumvents the point at which LeTx represses GR has the potential as use as a therapeutic. One such drug is rolipram, which is a phosphodiesterase inhibitor but has been show to activate GR. These data show that rolipram is unable to prevent the LeTx repression of dex induced TAT activity in HTC.

### Example 27

### Rolipram Does Not Prevent LeTx Toxicity in Raw264.7 Cell Lines

This example demonstrates that rolipram does not prevent LeTx toxicity in Raw264.7 cell lines. Raw264.7 cells were grown in DMEM and exposed to increasing concentrations of LF in the presence of 500 ng/ml PA for 24 hours. In some cases the cells were pre-treated for two hours, co-treated or pre- and co-treated with 10 μM rolipram. MTT assay was performed at the end of the 24 hours and the percent cell survival is shown as the percentage cells surviving compared to cells that have not been exposed to LeTx. Thus, rolipram is unable to prevent LeTx cytotoxicity in a sensitive macrophage cell line.

### Example 28

### The Extent of LeTx Repression of Progesterone-Induced GR, PR-B and GR/PR Chimera Transactivation in cos7 Cells is Dependent on the Promoter Construct

This example demonstrates that the extent of LeTx repression of progesterone-induced GR, PR-B and GR/PR chimera transactivation in cos7 cells is dependent on the promoter construct. Cos7 cells were transfected with the receptor expression plasmids for GR, PR-B, and the two chimeras GR/PR and PR/GR, and with the reporter constructs (GRE)₂ TK luc (solid symbols) or PLTR luc (open symbols) and subsequently treated with 100 nM progesterone and increasing concentrations of LF in the presence of 500 ng/ml PA. Relative luciferase induction is shown.

LeTx represses GR transactivation on a (GRE)2TK luc promoter by 40-50% and represses PR-B transactivation on a pLTR-luc promoter by 70% (see above). This difference was examined using chimeras of GR/PR and changing the promoters, since both GR and PR-B are able to activate both of these promoter constructs. These data show that the difference in repression (40-50% versus 70% repression) is a function of the promoter context. All of the receptors repress the pLTR-luc promoter to a greater extent than the (GRE)2TK luc promoter.

### Example 29

### The Extent of LeTx Repression of Dexamethasone-Induced GR and GR/PR Chimera Transactivation in cos7 Cells is Dependent on the Promoter Construct

This example demonstrates that the extent of LeTx repression of Dexamethasone-induced GR and GR/PR chimera transactivation in cos7 cells is dependent on the promoter construct. Cos7 cells were transfected with the receptor expression plasmids for GR, and the two chimeras GR/PR and PR/GR, and with the reporter constructs (GRE)₂ TK luc solid symbols) or PLTR luc (open symbols) and subsequently treated with 100 nM Dexamethasone and increasing concentrations of LF in the presence of 500 ng/ml PA. Relative luciferase induction is shown.

LeTx represses GR transactivation on a (GRE)2TK luc promoter by 40-50% and represses PR-B transactivation on a pLTR-luc promoter by 70% (see above). This difference was examined using chimeras of GR/PR and changing the promoters, since both GR and PR-B are able to activate both of these promoter constructs. These data show that the difference in repression (40-50% versus 70% repression) is a function of the promoter context. All of the receptors repress the pLTR-luc promoter to a greater extent than the (GRE)2TK luc promoter.

### Example 30

### Extent of LeTx Repression of Aldosterone-, Corticosterone, and Dexamethasone-Induced GR/MR Chimera Transactivation in Cos7 Cells

This example demonstrates the extent of LeTx repression of aldosterone-, corticosterone, and dexamethasone-induced GR/MR chimera transactivation in cos7 cells. Cos7 cells were transfected with the receptor expression plasmids for GR, MR and various GR/MR chimeras and with the reporter constructs (GRE)₂ TK, luc (FIGS. 21A, 21C, and 21E) or pltruc (FIGS. 21B and 21D) and subsequently treated with 100 nM aldosterone (FIGS. 21A and 21B), 1 μM corticosterone (FIGS. 21C and 21D), or 100 nM dexamethasone and increasing concentrations of LF in the presence of 500 ng/ml PA. Relative luciferase induction is shown.

The rational behind this example is that using chimeras of MR and GR on the (GRE)2TK promoter will help us determine which region of the GR is required for the repression. This indicates that the end of the N-terminal domain and the DNA binding domain (amino acids 404-525) is required for LeTx repression.

### Example 31

### Method for Diagnosis

A method for diagnosis of a subject having or at risk of having a disorder associated with a cofactor of a nuclear hormone receptor is disclosed herein. The disorder can be associated with an increase or a decrease in the cofactor of the nuclear receptor, as compared to a subject not affected by the disorder. In one embodiment, the method is used to identify an individual at risk for toxic effects of exposure to pathogenic bacteria, for example anthrax. The method includes obtaining a sample from the subject that includes a cofactor of a nuclear hormone receptor. The sample is contacted with the bacterial product. An increase in the binding of the bacterial product indicates that the co-factor is increased as compared to a normal subject (a subject not affected with the disorder). A decrease in the binding of the bacterial product indicates that the co-factor is decreased as compared to a normal subject. Thus, the binding of bacterial product to the sample indicates that the subject has the disorder.

In one example, the bacterial product is directly labeled. In another example, an antibody is utilized that specifically binds the bacterial product. These antibodies are of use, for example, in immunoassays in which they can be utilized in liquid phase or bound to a solid phase carrier. In addition, the antibodies in these immunoassays can be detectably labeled in various ways. Examples of types of immunoassays which can utilize antibodies are competitive and non-competitive immunoassays in either a direct or indirect format. Examples of such immunoassays are the radioimmunoassay (RIA) and the sandwich (immunometric) assay. Detection of the bound bacterial product using the antibodies can be carried out utilizing a variety of immunoassays, including immunohistochemical assays on physiological samples. Those of skill in the art will know, or can readily discern, other immunoassay formats without undue experimentation.

In one example, the bacterial product is LeTx or LF, and the disorder is associated with increased or decreased expression of GR, PR, or β-ER. Certain strains of rodents show enhanced susceptibility to lethal effects of exposures to anthrax. As demonstrated herein, differences in characteristics of a nuclear hormone receptor in these animals is indicative that they are susceptible to anthrax. Thus, in order to determine if an individual is highly susceptible or highly resistant to an anthrax infection, a sample can be obtained from the individual that includes nuclear hormone receptors. The sample is contacted with LeTx or LF, and the binding of the toxin to the sample is assessed. A change in the binding of LeTx or LF, as compared to a normal subject, can be used to demonstrate that the subject is either highly susceptible or highly resistant to an anthrax infection.

Although the foregoing disclosure has been described in detail by way of example for purposes of clarity of understanding, it will be apparent to the artisan that certain changes and modifications may be practiced within the scope of the appended claims which are presented by way of illustration not limitation. In this context, various publications have been cited within the foregoing disclosure for economy of description

What is claimed is:

1. A method for identifying an agent that modulates the activity of a nuclear hormone receptor comprising the steps of:
providing a viable cell that expresses a nuclear hormone receptor and a nuclear hormone receptor substrate, reporter construct, or both, wherein expression of the substrate reporter construct is detectable and provides a measurement of nuclear hormone receptor pathway activity;
contacting a test cell with a test agent and an isolated bacterial product;
contacting a control cell with the isolated bacterial product in the absence of the agent;
detecting and comparing nuclear hormone receptor activity between the test and control cell to identify a test agent that interacts with the nuclear hormone receptor and modulates the activity of the nuclear hormone receptor by the bacterial product.

2. The method of claim 1, wherein the nuclear hormone receptor is selected from a glucocorticoid receptor (GR), androgen receptor (AR), mineralocorticoid receptor (MR), progestin receptor (PR), estrogen receptor (ER), thyroid hormone receptor (TR), vitamin D receptor (VDR), retinoid receptor (RAR or RXR), peroxisome receptor (XPAR or PPAR), icosanoid receptor (IRs), steroid receptor and thyroid receptor.

3. The method of claim 2, wherein the nuclear hormone receptor is GR.

4. The method of claim 2, wherein the nuclear hormone receptor is PR.

5. The method of claim 1, wherein the bacterial product is a bacterial wall protein, soluble bacterial protein, or lipopolysaccharide.

6. The method of claim 1, wherein the bacterial product is a bacterial toxin that is not endotoxin.

7. The method of claim 6, wherein the bacterial toxin elicits one or more symptoms of a toxic effect, inflammatory response, stress, shock, chronic sequelae, autoimmunity, or mortality in a susceptible host infected with a bacterium that produces the toxin.

8. The method of claim 6, wherein the bacterial toxin exhibits metalloprotease activity.

9. The method of claim 8, wherein the bacterial toxin is anthrax lethal factor (LF) or lethal toxin (LeTx) or a metalloenzyme of Clostridium tetanus or C. botulinum bacteria.

10. The method of claim 1, wherein the bacterial product is a bacterial antigen.

11. The method of claim 10, wherein the bacterial antigen is a pyrogenic toxin superantigen (PTSAg).

12. The method of claim 1, wherein the agent exerts its effect on the nuclear hormone receptor is through a mechanism other than inhibition of a MEK1 or MAPKK pathway.

13. The method of claim 1, wherein the agent is a genetically engineered or chemically modified variant or mimetic of the bacterial product, a drug, or a cofactor for the nuclear hormone receptor.

14. The method of claim 1, wherein the agent is effective following administration to a mammalian subject to reduce one or more inflammatory and/or autoimmune symptoms that can accompany exposure to the bacterial product or infection by a pathogen expressing the product.

15. The method of claim 1, wherein the isolated bacterial product alters the activity of the nuclear hormone receptor and does not alter number of nuclear hormone receptors on the viable cell.

16. A method for identifying an agent that inhibits nuclear hormone receptor repression by a bacterial product comprising the steps of:
providing viable cells that express a nuclear hormone receptor and a nuclear hormone substrate, a reporter construct, or both, wherein expression of the substrate, the reporter construct or both is detectable and provides a measurement of nuclear hormone receptor pathway activity;
contacting test cells cells with a test agent and a bacterial product;
contacting control cells with a bacterial product;
detecting and comparing nuclear hormone receptor pathway activity between the test and control cells to identify a test agent that inhibits repression of the receptor pathway activity by the bacterial product.

17. The method of claim 16, wherein the bacterial product is anthrax lethal factor (LF) or lethal toxin (LeTx).

18. The method of claim 17, wherein the agent that inhibits or blocks anthrax lethal factor (LF) or lethal toxin (LeTx) repression of nuclear hormone receptor activity is a cofactor for the nuclear hormone receptor.

19. The method of claim 18, wherein the cofactor is a coactivator for the nuclear hormone receptor.

20. The method of claim 18, wherein the nuclear hormone receptor is GR.

21. The method of claim 18, wherein the nuclear hormone receptor is PR.

22. The method of claim 18, wherein the nuclear hormone receptor is estrogen receptor-α (ER-α).

23. The method of claim 18, wherein the effective agent is a genetically engineered or chemically modified variant or mimetic of LF or LeTx, a drug, or a cofactor for the nuclear hormone receptor.

24. The method of claim 24, wherein the effective agent is a co-activator for the nuclear hormone receptor.

25. A method for identifying an active protein or other macromolecule from a cell expressesing a nuclear hormone receptor, wherein the active protein or other macromolecule interacts with a bacterial product that modulates nuclear hormone receptor pathway activity, comprising the steps of:
exposing the bacterial product to a lysate or other biological sample from the cell expressing the nuclear hormone receptor under conditions to allow for binding of the bacterial product to the active protein or other macromolecule;
contacting the bacterial product with a binding partner that provides for isolation or identification of the bacterial product bound to the active protein or other macromolecule;
detecting a bound complex of the bacterial product with the active protein or other macromolecule; and
identifying the active protein or other macromolecule bound in the complex.

26. The method of claim 25, wherein the binding partner is a polyclonal or monoclonal antibody that binds the bacterial product.

27. The method of claim 32 which comprises an immunoprecipitation assay.

28. The method of claim 25, wherein the active protein or other macromolecule bound in the complex is identified before separation from the complex, or following an additional step to separate the active protein or other macromolecule from the complex.

29. The method of claim 31, wherein the active protein or other macromolecule bound in the complex is identified by Western blotting and/or mass spectroscopy.

30. A method for alleviating or preventing one or more symptoms of a bacterial disease, inflammatory reaction, or autoimmune response in a mammalian subject comprising administering an effective amount of an agonist or antagonist of a nuclear hormone receptor selected according to the method of claim 1.

31. A method for alleviating or preventing one or more symptoms of a bacterial disease, inflammatory reaction, or autoimmune response in a mammalian subject comprising administering an effective amount of an agent that inhibits or enhances modulation of a nuclear hormone receptor by a bacterial product.

32. A method for alleviating or preventing one or more symptoms of anthrax disease and/or an associated inflammatory reaction, or autoimmnune response, in a mammalian subject comprising administering an effective amount of an effective agent that inhibits, blocks, or enhances modulation of activity of one or more nuclear hormone receptor(s) by a anthrax lethal factor (LF) or lethal toxin (LeTx) or an analog, variant, derivative, or mimetic thereof.

33. A method for alleviating or preventing one or more symptoms of a bacterial disease, inflammatory reaction, or autoimmune response in a mammalian subject comprising administering an effective amount of a cofactor that is an agonist or antagonist of a nuclear hormone binding receptor.

34. A pharmaceutical composition for alleviating or preventing one or more symptoms of a bacterial disease, inflammatory reaction, or autoimmune response in a mammalian subject comprising an effective amount of an agonist or antagonist of a nuclear hormone receptor selected according to the method of claim 1.

35. A pharmaceutical composition for alleviating or preventing one or more symptoms of a bacterial disease, inflammatory reaction, or autoimmune response in a mammalian subject comprising an effective amount of an agent that inhibits or enhances modulation of a nuclear hormone receptor by a bacterial product.

36. The method of claim 35, wherein the agent is a cofactor of the nuclear hormone receptor.

37. A pharmaceutical composition for alleviating or preventing one or more symptoms of anthrax disease and/or an associated inflammatory reaction, or autoimmune response, in a mammalian subject comprising an effective amount of an effective agent that inhibits, blocks or enhances modulation of activity of one or more nuclear hormone receptor(s) by an anthrax lethal factor (LF) or lethal toxin (LeTx) or an analog, variant, derivative, or mimetic thereof.

38. A composition comprising a recombinantly or chemically modified analog, fragment or derivative of a bacterial product that exhibits substantially reduced or increased activity as a modulator of nuclear hormone receptor activity compared to a native or wild-type counterpart bacterial product.

39. The composition of claim 38, wherein the composition elicits an immune response against the native or wild-type counterpart bacterial product in a mammalian subject

40. The immunogenic composition of claim 38, wherein said analog, fragment or derivative comprises a mutant variant, truncated fragment, or chemically modified derivative of an anthrax lethal factor (LF) or lethal toxin (LeTx).

41. The immunogenic composition of claim 40, wherein said LF or LeTx variant, fragment or derivative exhibits substantially reduced or increased activity for GR and/or PR repression.

42. The immunogenic composition of claim 40, wherein said LF or LeTx variant, fragment or derivative exhibits substantial activity as an immunogen, and/or inhibits, blocks, or enhances nuclear hormone repression activity by native LF or LeTx.

43. The immunogenic composition of claim 38, wherein said analog, fragment or derivative is characterized by a reduction or increase in a level of nuclear hormone modulation activity of at least 30% compared to repressor modulation activity of a corresponding native bacterial product.

44. A composition comprising a recombinantly or chemically modified analog, fragment or derivative of a bacterial product that inhibits, blocks, or enhances an interaction of a corresponding native bacterial product with a nuclear hormone receptor.

45. A method for identifying an agent of use in treating anthrax, comprising:
providing viable cells that express a receptor selected from the group consisting of a glucocorticoid receptor, an estrogen receptor α (ER-α), and a progresterone receptor B (PR-B) and a nucleic acid comprising a responsive element selected from the group consisting of a glucocorticoid receptor responsive element, an estrogen receptor α (ER-α) responsive element, and a progresterone receptor B (PR-B) responsive element, respectively, wherein the responsive element is operably linked to a nucleic acid encoding a polypeptide, wherein expression of the polypeptide is detectable and provides a measurement of the activity of the glucocorticoid responsive element;
contacting test cells with a test agent and anthrax lethal toxin (LeTx);
detecting expression of the polypeptide, wherein increased expression of the polypeptide as compared to a control identifies the agent as of use in treating anthrax.

46. The method of claim 45, wherein the control is a test cell contacted with anthrax lethal toxin in the absence of the agent.

47. The method of claim 45, wherein the receptor comprises a glucocorticoid receptor and wherein the responsive element is a glucocorticoid responsive element.

48. The method of claim 45, wherein the receptor comprises a estrogen receptor α (ER-α) and the responsive element comprises a estrogen receptor α (ER-α) responsive element.

49. The method of claim 45, wherein the receptor is a progesterone receptor B (PR-b) and the responsive element comprises a progesterone receptor B responsive element.

50. A agonist of the glucocorticoid receptor, an estrogen receptor α (ER-α), and a progresterone receptor B (PR-B) in the manufacture of a medicament for the treatment of anthrax.

51. A method for treating an anthrax infection, comprising administering to a subject infected with anthrax or at risk of infection with anthrax a therapeutically effective amount of an agent that affects the activity of the glucocorticoid receptor, an estrogen receptor a (ER-α), and a progresterone receptor B (PR-B), thereby treating the anthrax infection.


SEQUENCE LISTING (1 sequence)

SEQ ID NO: 1 - protein, 13 aa, Human immunodeficiency virus type 1
GRKKRRQRRRPPQ